(19) United States
(12) Patent Application Publication
(10) Pub. No.: US 2024/0209084 A1
(43) Pub. Date: Jun. 27, 2024

(54) BI-SPECIFIC ANTIBODIES FOR USE IN PRODUCING ARMED IMMUNE CELLS

(71) Applicant: CYTOARM CO. LTD., Taipei City (TW)

(72) Inventors: Kuo-Hsiang CHUANG, Taipei City (TW); Michael CHEN, Taipei City (TW); Yi-Jou CHEN, Taipei City (TW)

(73) Assignees: Michael CHEN, Taipei City (TW); CYTOARM CO. LTD., Taipei City (TW)

(21) Appl. No.: 17/913,775

(22) PCT Filed: Mar. 23, 2021
(86) PCT No.: PCT/US2021/023655

§ 371 Date: Sep. 22, 2022

Related U.S. Application Data
(60) Provisional application No. 62/993,080, filed on Mar. 23, 2020.

(51) Int. Cl. C07K 16/28; A61K 39/00; A61P 35/02; C07K 16/30; C07K 16/32; C07K 16/40; C12N 5/0783; C12N 15/85

(52) U.S. Cl. CPC C07K 16/2809; A61P 35/02; C07K 16/2803; C07K 16/2827; C07K 16/2863; C07K 16/2878; C07K 16/2887; C07K 16/2896; C07K 16/30; C07K 16/3007; C07K 16/3069; C07K 16/3084; C07K 16/32; C07K 16/40; C12N 5/0637; C12N 15/85; A61K 2039/5156; C07K 2317/31; C07K 2317/55; C07K 2317/565; C07K 2317/622; C07K 2317/64

(57) ABSTRACT

Bispecific antibodies capable of binding to CD3 and a tumor associated antigen. immune cells armed with such bispecific antibodies. and therapeutic uses thereof in cancer treatment. Also provided herein are methods for producing immune cells armed with the bispecific antibodies.

### CROSS REFERENCE TO RELATED APPLICATIONS

This application claims the benefit of the filing date of U.S. Provisional Application No. 62/993,080, filed Mar. 23, 2020, the entire contents of which is incorporated by reference herein.

### BACKGROUND OF THE INVENTION

Cancer is a disease characterized by abnormal cells that divide uncontrollably and have the ability to infiltrate and destroy normal tissue and/or organ of a subject. Cancer is the second leading cause of death globally, and is responsible for an estimated 9.6 million deaths in 2018, in which the most common cancers include, lung cancer (about 2.09 million cases), breast cancer (about 2.09 million cases), colorectal cancer (about 1.80 million cases), prostate cancer (about 1.28 million cases), skin cancer (about 1.04 million cases), and gastric cancer (about 1.03 million cases).

Treatments for cancers may vary with the type of cancer and how advanced it is. Conventional treatments for cancers include surgery, radiation therapy, and chemotherapy. Such treatments usually cause a variety of complications or side effects, such as infection, blood clot, bleeding, nausea and vomiting, diarrhea, nerve or muscle damage, incontinence, and sex and fertility issues. Immunotherapy provides an alternative strategy for cancer treatment that aims at specifically stimulating immune responses of a subject against cancer cells via, for example, blocking immune checkpoints, or enhancing the ability of immune cells (e.g., T cells or B cells) to target and destroy cancer cells. Serious adverse effects associated with immunotherapy-medicated overstimulation or non-specific toxicity have been reported in cancer patients, including neurotoxicity, cytokine release syndrome (CRS), allergy, organ inflammation, and autoimmune disorders.

It is therefore of great importance to develop efficient cancer treatment specifically targeting cancer cells without affecting normal cells and/or tissues.

### SUMMARY OF THE INVENTION

The present disclosure is based on the development of bispecific antibodies (BsAbs) capable of binding to CD3 (e.g., human CD3) and a tumor associated antigen (TAA). Such BsAbs are capable of attaching to the surface of CD3-positive immune cells via binding of the anti-CD3 moiety in the BsAb to the cell surface CD3 to produce armed immune cells.

Accordingly, the present disclosure features, in some aspects, a bi-specific antibody, comprising: (a) a first antigen binding fragment that binds human CD3, and (b) a second antigen binding fragment that binds a tumor associated antigen (TAA). The first antigen binding fragment comprises (i) a first heavy chain comprising a first heavy chain variable region (V_{H}) and (ii) a first light chain comprising a first light chain variable region (V_{L}). In some embodiments, the first V_{H} comprises the same heavy chain complementary determining regions (CDRs) as a first reference antibody. In other embodiments, the first V_{H} comprises or no more than 5 amino acid variations in CDRs relative to the first reference antibody. Alternatively or in addition, the first V_{L} may comprise the same light chain CDRs as the first reference antibody. In other embodiments, the first V_{L} may comprise no more than 5 amino acid variations in the CDRs relative to the first reference antibody. In some examples, the first reference antibody is CTA.02. In some examples, the first reference antibody is CTA.03. In other examples, the first reference antibody is CTA.04. In yet other examples, the first reference antibody is CTA.05. Structural information of these exemplary reference antibodies are provided in Table 1 below. In some examples, the first heavy chain and the first light chain comprise the same V_{H} and V_{L} as the first reference antibody.

The second antigen binding fragment comprises a second heavy chain comprising (i) a second heavy chain variable region (V_{H}), and (ii) a second light chain comprising a second light chain variable region (V_{L}). The second antigen binding fragment binds a TAA. Examples include CD20, CD19, EGFR, HER2, PSMA, CEA, EpCAM, FAP, PD-L1, CD38, CD33, cMET, CD47, TRAIL-R2, mesothelin, or GD2. In some instances, the second V_{H} comprises the same heavy chain complementary determining regions (CDRs) as a second reference antibody. Alternatively, the second V_{H} may comprise no more than five amino acid variations in the CDRs relative to the second reference antibody. Alternatively or in addition, the second V_{L} may comprise the same light chain CDRs. In other examples, the second V_{L} may comprise no more than 5 amino acid variations in the CDRs relative to the second reference antibody. In some instances, the second reference antibody is CTAT.01, CTAT.02, CTAT.03, CTAT.04, CTAT.05, CTAT.06, CTAT.07, CTAT.08, CTAT.09, CTAT.10, CTAT.11, CTAT.12, CTAT.13, CTAT.14, CTAT.15, or CTAT.16. See Table 2 below. In some examples, the second antigen binding fragment comprises the same V_{H} and same V_{L} as the second reference antibody.

In some embodiments, the first antigen binding fragment is a Fab fragment and the second antigen binding fragment is a single chain variable fragment (scFv). In some examples, the Fab fragment comprises the first heavy chain, which comprises the first V_{H} and a CH1 fragment, and the first light chain, which comprises the first V_{L} and a light chain constant region. In specific examples, the Fab fragment may comprise the first heavy chain and the first light chain, which respectively comprise the amino acid sequences of (a) SEQ ID NO:10 and SEQ ID NO: 11, (b) SEQ ID NO: 23 and SEQ ID NO: 24, 25, or 228, (c) SEQ ID NO: 35 and SEQ ID NO: 36, or (d) SEQ ID NO: 46 and SEQ ID NO: 47. In some examples, the scFv of the second antigen binding fragment comprises the amino acid sequence of any one of SEQ ID NOs: 254-271.

In some instances, the scFv is linked to the CH1 fragment, w optionally is via a peptide linker. Alternatively, the scFv is linked to the light chain constant region, optionally via a peptide linker. For example, the bi-specific antibody may comprise a first polypeptide comprising the first light chain and a second polypeptide comprising, from N-terminus to C-terminus, the first heavy chain, the peptide linker, and the scFv. Examples include any one of SEQ ID NOs: 229-248. Such a bi-specific antibody may comprise a second polypeptide comprising an amino acid sequence selected from the group consisting of SEQ ID NOs: 24, 25, and 228. See Table 3 below.

In other instances, the first antigen binding fragment is a single chain variable fragment (scFv) and the second antigen binding fragment is a Fab fragment. The scFv may comprise the amino acid sequence of any one of SEQ ID NOs: 250-253. In some examples, the Fab fragment comprises the second heavy chain, which comprises the second V_{H} and a CH1 fragment, and the second light chain, which comprises the second V_{L} and a light chain constant region. In specific examples, the Fab fragment comprises the first heavy chain and the first light chain, which respectively comprise the amino acid sequences of (1) SEQ ID NO:57 and SEQ ID NO: 58, (2) SEQ ID NO: 72 and SEQ ID NO: 73, (3) SEQ ID NO: 83 and SEQ ID NO: 84, (4) SEQ ID NO: 94 and SEQ ID NO: 95, (5) SEQ ID NO:105 and SEQ ID NO:106, (6) SEQ ID NO: 116 and SEQ ID NO:117, (7) SEQ ID NO: 127 and SEQ ID NO:128, (8) SEQ

ID NO:138 and SEQ ID NO:139, (9) SEQ ID NO:149 and SEQ ID NO:150, (10) SEQ ID NO:160 and SEQ ID NO:161, (11) SEQ ID NO: 171 and SEQ ID NO:172, (12) SEQ ID NO: 182 and SEQ ID NO:183, (13) SEQ ID NO: 193 and SEQ ID NO: 194, (14) SEQ ID NO:204 and SEQ ID NO:205, (15) SEQ ID NO:215 and SEQ ID NO:216, or (16) SEQ ID NO:226 and SEQ ID NO:227.

In some examples, the scFv is linked to the CH1 fragment, optionally via a peptide linker. Alternatively, the scFv is linked to the light chain constant region, optionally via a peptide linker. Any of the peptide linker may be at least 5 amino acids in length.

In yet other instances, both the first antigen binding fragment and the second antigen binding fragment are scFv antibodies. In some examples, the bi-specific antibody comprises a polypeptide comprising the two scFv antibodies.

In other aspects, the present disclosure provides an armed immune cell, comprising an immune cell that expresses surface CD3, and any of the bi-specific antibodies disclosed herein (e.g., those exemplified in Tables 1-3). The armed immune cell displays the bi-specific antibody on the surface via interaction between the first antigen binding fragment in the bi-specific antibody and the CD3 expressed by the immune cell. In some embodiments, the immune cell is a T cell, a B cell, a monocyte, a macrophage, or a combination thereof. In some instances, the T cell can be a CD4+ T cell, a CD8+ T cell, a regulatory T cell, or a natural killer T cell. In some examples, the immune cell is a human immune cell, for example, immune cells derived from a human donor.

In addition, provided herein is a method of producing the armed immune cell as disclosed herein. The method may comprise cultivating a cell population comprising the immune cells in the presence of the bi-specific antibody as disclosed herein to allow for binding of the bi-specific antibody to the immune cells, thereby producing the armed immune cell. The armed immune cells produced by any of the methods disclosed herein are also within the scope of the present disclosure.

In some embodiments, the cell population comprises T cells, B cells, monocytes, macrophages, or a combination thereof. In some examples, the cell population comprises peripheral blood mononuclear cells (PBMCs) or immune cells derived from stem cells in vitro. The stem cells may be hematopoietic stem cells, umbilical cord blood stem cells, or induced pluripotent stem (iPS) cells.

In some embodiments, the cultivating step is performed in a culture medium comprising a cytokine, which optionally comprises interleukin 2 (IL-2), interleukin 7 (IL-7), transforming growth factor-beta (TGF-β), or a combination thereof.

Further, provided herein is a method for treating cancer, comprising administering to a subject in need thereof an effective amount of a population of any of the armed immune cells disclosed herein. The subject has or suspected of having a cancer that is positive with the TAA, to which the second antigen binding fragment of the bi-specific antibody binds. In some embodiments, the subject is a human cancer patient. In some embodiments, the armed immune cells are autologous to the subject. Alternatively, the armed immune cells are allogenic to the subject. Exemplary cancers include, but are not limited to, melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, leukemia, lymphoma, and myeloma.

In other aspects, the present disclosure features a nucleic acid or a set of nucleic acids (two nucleic acid molecules), which encodes or collectively encodes any of the bi-specific antibodies disclosed herein. In some examples, the nucleic acid or set of nucleic acids is a vector or a set of vectors, for examples, expression vector(s). Host cells (e.g., a bacterial cell, a yeast cell, or a mammalian cell) comprising any of the nucleic acid or set of nucleic acids disclosed herein are also within the scope of the present disclosure.

In addition, the present disclosure features a method for producing a bi-specific antibody, comprising: (i) culturing a host cell as disclosed herein under conditions allowing for expressing of the bi-specific antibody; and (ii) harvesting the bi-specific antibody.

Also within the scope of the present disclosure are armed immune cells as disclosed herein for use in cancer treatment or use of any of the armed immune cells for manufacturing a medicament for use in treating a target cancer.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1N are schematic diagrams of exemplary bi-specific antibody formats. FIGS. 1A-1D: structures of anti-CD3 Fab/anti-TAA scFv bi-specific formats. FIGS. 1E-1H: structures of anti-CD3 scFv/anti-TAA Fab bi-specific formats. FIGS. 1I-1L: structures of anti-CD3 scFv/anti-TAA scFv bi-specific formats. FIG. 1M: structure of anti-CD3 knob/anti-TAA hole bi-specific antibody formats, which comprises a monovalent anti-CD3 antibody and a monovalent anti-TAA antibody. FIG. 1N: anti-CD3 knob/anti-TAA scFv hole antibody, which comprises a monovalent anti-CD3 antibody and a monovalent anti-TAA scFv-Fc fusion protein.

FIGS. 2A-2E include schematic diagrams of DNA constructs for expressing the illustrated recombinant bi-specific antibodies. FIG. 2A: exemplary constructs for expressing anti-CD3Fab/anti-TAA scFv bi-specific antibodies. FIG. 2B: exemplary constructions for expressing anti-CD3scFv/anti-TAA Fab bi-specific antibodies. FIG. 2C: exemplary constructs for expressing anti-CD3 scFv/anti-TAA scFv bi-specific antibodies. FIG. 2D: exemplary constructs for expressing anti-CD3 knob/anti-TAA hole bi-specific antibodies. FIG. 2E: exemplary constructs for expressing anti-CD3 knob/anti-TAA scFv hole antibody.

FIG. 3 is a chart showing binding affinity of exemplary bi-specific antibodies to T cells as measured by flow cytometry.

FIG. 4 is a chart depicting the cytotoxic effect of T cells armed with exemplary bi-specific antibodies or activated by OKT3 antibody against HT-29 cancer cells.

FIG. 5 is a chart depicting a time course of the levels of exemplary bi-specific antibodies on the surface of T cells.

FIGS. 6A-6B include photos showing expression and assembly of the exemplary bi-specific antibodies as indicated by SDS-PAGE under non-reducing conditions (FIG. 6A) and reducing conditions (FIG. 6B). Estimated molecular weight: intact BsAb=95 kDa, heavy chain=60 kDa, and light chain=25 kDa. Lane 1: protein marker, Lane 2: CTA02Fab/CTAT02scFv, Lane 3: CTA04Fab/CTAT02scFv. Lane 4: CTA03Fab/CTAT02scFv, and Lane 5: CTA05Fab/CTAT02scFv.

FIGS. 7A-7F include photos showing expression and assembly of the exemplary anti-CD3 Fab/anti-tumor bi-specific antibodies as indicated via SDS-PAGE under reducing or non-reducing conditions. Estimated molecular weight: intact BsAb=95 kDa. FIGS. 7A-7B: CTA03Fab/anti-TAA scFv bi-specific antibodies under non-reducing conditions. FIGS. 7C-7D: CTA03Fab/anti-TAA scFv bi-specific antibodies under reducing conditions. FIGS. 7E-7F: anti-CD3 scFv/CTAT03Fab bispecific antibodies under non-reducing and reducing conditions, respectively.

FIG. 8 is a diagram showing the binding activity of various bi-specific antibodies as indicated to T cells and to tumor cells. CD3⁺ T cells (Jurkat) and CD19⁺ B cell lymphoma (Raji) were incubated separately with exemplary bi-specific antibodies CTA02Fab/CTAT02scFv, CTA03Fab/CTAT02scFv, CTA04Fab/CTAT02scFv, and CTA05Fab/CTAT02scFv BsAbs, and then analyzed with FITC conjugated Goat anti-Human IgG Fab antibody and flow cytometer.

FIGS. 9A-9L include diagrams showing binding activity of exemplary anti-CD3 Fab/anti-tumor scFv bi-specific antibodies as indicated to T cells and to tumor cells. FIG. 9A: CTA03Fab/CTAT02scFv binding to CD3⁺ T cells (Jurkat) and CD19⁺ B cell lymphoma (Raji). FIG. 9B: CTA03Fab/CTAT03scFv binding to CD3⁺ T cells (Jurkat) and EGFR⁺ triple negative breast cancer (MDA-MB-231). FIG. 9C: CTA03Fab/CTAT04scFv binding to CD3⁺ T cells (Jurkat) and HER2⁺ breast cancer (MCF7/HER2). FIG. 9D: CTA03Fab/CTAT05scFv binding to CD3⁺ T cells (Jurkat) and PSMA⁺ Prostate cancer (LNCaP). FIG. 9E: CTA03Fab/CTAT07scFv binding to CD3⁺ T cells (Jurkat) and EpCAM⁺ Prostate cancer (LNCaP). FIG. 9F: CTA03Fab/CTAT08scFv binding to CD3⁺ T cells (Jurkat) and FAP⁺ mouse fibroblasts cell(3T3/FAP). FIG. 9G: CTA03Fab/CTAT09scFv binding to CD3⁺ T cells (Jurkat) and PDL1⁺ triple negative breast cancer (MDA-MB-231). FIG. 9H: CTA03Fab/CTAT10scFv binding to CD3⁺ T cells (Jurkat) and CD38⁺ B cell lymphoma (Raji). FIG. 9I: CTA03Fab/CTAT1 1scFv binding to CD3⁺ T cells (Jurkat) and CD33⁺ human acute myeloid leukemia (HL-60). FIG. 9J: CTA03Fab/CTAT12scFv binding to CD3⁺ T cells (Jurkat) and HGFR⁺ human lung carcinoma (A549). FIG. 9K: CTA03Fab/CTAT13scFv binding to CD3⁺ T cells (Jurkat) and CD47⁺ breast cancer (MCF7/HER2). FIG. 9L: binding of BsAbs CTA02scFv/CTAT03Fab CTA03scFv/CTAT03Fab, CTA04scFv/CTAT03Fab, and CTA05scFv/CTAT03Fab to CD3⁺ T cells (Jurkat) (upper panel) and EGFR+ colon cancer (HT-29) (bottom panel). Samples were analyzed with FITC conjugated Goat anti-Human IgG Fab antibody and flow cytometry.

FIG. 10 is a chart showing the retention ability of exemplary BsAbs on T cell surface. Human T cells were incubated with variant Anti-CD3Fab/anti-CD19scFv BsAbs with a Fab of 4 different anti-CD3 antibody (CTA01Fab/CTAT02scFv, CTA02Fab/CTAT02scFv, CTA03Fab/CTAT02scFv, and CTA05Fab/CTAT02scFv) for 1 hr, and then were cultured in medium for 5 min, 24, 48, and 72 hr. After the culture, the cells were stained with FITC conjugated Goat anti-Human IgG Fab antibody and the retention of BsAb on T cell surface was analyzed using flow cytometry.

FIGS. 11A and 11B include diagrams showing formation of T cells armed with exemplary BsAbs disclosed herein. FIG. 11A: OKT3, CTA01Fab/CTAT02scFv, and CTA02Fab/CTAT02scFv, from left to right. FIG. 11B: CTA03Fab/CTAT02scFv (left) and CTA05Fab/CTAT02scFv (right). PBMCs were cultured in the presence of OKT3 or the various BsAbs. The cell cultures were then stained with FITC conjugated CD8 antibody and PE conjugated goat anti-Human IgGFab, and then analyzed using flow cytometry.

FIGS. 12A-12D include diagrams showing formation of T cells armed with exemplary BsAbs as indicated. FIG. 12A: OKT3, CTA03Fab/CTAT03scFv, and CTA03Fab/CTAT04scFv (top panel, left to right), and CTA03Fab/CTAT09scFv, CTA03Fab/CTAT010scFv, and CTA03Fab/CTAT1 1scFv (bottom panel, left to right). FIG. 12B: CTA03Fab/CTAT05scFv, CTA03Fab/CTAT07scFv, and CTAFab/CTAT08scFv (top panel, left to right), and CTA03Fab/CTAT12scFv and CTA03Fab/CTAT13scFv (bottom, left to right). FIG. 12C: OKT3, CTA01scFv/CTAT03Fab, and CTA02scFv/CTA03Fab (left to right). FIG. 12D: CTA03scFv/CTAT03Fab, CTA04scFv/CTAT03Fab, and CTA05scFv/CTAT03Fab (left to right). The cell cultures were stained with anti-CD8 antibody and goat anti-Human IgG Fab, and then analyzed using flow cytometry.

FIGS. 13A-13B include charts showing cytotoxicity activity of T cells induced by OKT3 antibody or armed with exemplary bi-specific antibodies as indicated against tumor cells. FIG. 13A: anti-CD3Fab/anti-CD19scFv BsAbs against B cell lymphoma. FIG. 13B: anti-CD3scFv/CTAT03Fab BsAbs against HT29 cells. Cells were cultured with OKT3 or the exemplary BsAbs as indicated. The cell cultures were then incubated with CD19⁺ B cell lymphoma (Raji) at several effector cell: target cell ratios (3:1, 5:1 and 10:1) for 18 hr. Tumor cell death was determined with CytoTox 96® Non-Radioactive Cytotoxicity Assay (Promega, G1780).

FIGS. 14A-14G include charges showing in vitro cytotoxic activity of anti-CD3Fab/anti-EGFRscFv BsAb armed T cells against cancer cells. FIG. 14A: CTA01Fab/CTAT03scFv or CTA03Fab/CTAT03scFv armed T cells against HT29 cells (EGFR+ colon cancer cells). FIG. 14B: CTA01Fab/CTAT03scFv or CTA03Fab/CTAT03scFv armed T cells against HCT-116 cells (EGFR+ colon cancer cells). FIG. 14C: antiCD3Fab/anti-HER2scFv (CTA03Fab/CTAT04scFv) armed T cells against HER2⁺ breast cancer cells (MCF-7/HER2). FIG. 14D: antiCD3Fab/anti-PSMAscFv (CTA03Fab/CTAT05scFv) armed T cells against PSMA⁺ prostate cancer cells (LNCaP). FIG. 14E: antiCD3Fab/anti-EpCAMscFv (CTA03Fab/CTAT07scFv) armed T cells against EpCAM⁺ prostate cancer cells (LNCaP). FIGS. 14F-14G: antiCD3Fab/anti-FAPscFv (CTA03Fab/CTAT08scFv) armed T cells against FAP⁻ mouse fibroblasts cells (3T3) (FIG. 14F) or FAP⁺ mouse fibroblasts cells (3T3/FAP) (FIG. 14G). OKT3-cultured T cells or armed T cells were cocultured with the cancer cells at several effector cell: target cell ratios (3:1, 5:1, and 10:1) for 18 hr. Tumor cell death was determined with CytoTox 96® Non-Radioactive Cytotoxicity Assay (Promega, G1780).

FIGS. 15A-15E include charts showing in vitro cytotoxic activities of exemplary antiCD3Fab/anti-TAA scFv BsAbs against corresponding cancer cells. FIG. 15A: antiCD3Fab/anti-PDL1scFv (CTA03Fab/CTAT09scFv) armed T cells against triple negative breast cancer cells (MDA-MB-231). FIG. 15B: antiCD3Fab/anti-CD38scFv (CTA03Fab/CTAT10scFv) armed T cells against CD38⁺ B cell lymphoma cells (Raji). FIG. 15C: antiCD3Fab/anti-CD33scFv (CTA03Fab/CTAT1 1scFv) armed T cells against CD33⁺ human acute myeloid leukemia cells (HL-60). FIG. 15D: antiCD3Fab/anti-HGFRscFv (CTA03Fab/CTAT12scFv) armed T cells against HGFR⁺ human lung carcinoma cells (A549). FIG. 15E: antiCD3Fab/anti-CD47scFv (CTA03Fab/CTAT13scFv) armed T cells against CD47⁺ breast cancer cells (MCF7/HER2). OKT3-cultured T cells or armed T cells were cocultured with the cancer cells at several effector cell: target cell ratios (3:1, 5:1, and 10:1) for 18 hr. Tumor cell death was determined with CytoTox 96® Non-Radioactive Cytotoxicity Assay (Promega, G1780).

FIGS. 16A-16C include diagrams showing in vivo therapeutic efficacy of exemplary anti-CD3Fab/anti-CD19scFv armed-T cell against lymphoma. SCID mice were i.v. inoculated with CD19⁺ B cell lymphoma cells (2.5×10⁶ cells/mice). After 3 days, T cell, CTA01Fab/CTAT02scFv armed-T cells sand CTA03Fab/CTAT02scFv armed-T cells were i.v. injected into the mice (5×10⁶ cells/mice, once a week for 4 times). FIG. 16A: Body weight. FIG. 16B: survival rate. FIG. 16C: incidence of hindlimb paralysis.

FIGS. 17A-17B include diagrams showing in vivo therapeutic efficacy of exemplary CTA03Fab/CTAT03scFv armed-T cells and CTA03Fab/CTAT04scFv armed-T cells against human triple-negative breast cancer. ASID mice were s.c. inoculated with clinical human breast tumor tissues. After 19 days, T cell, CTA03Fab/CTAT03scFv armed-T cells and CTA03Fab/CTAT04scFv armed-T cells were i.v. injected into the mice (5×10⁶ cells/mice, once a week for 4 times). FIG. 17A: Body weight. FIG. 17B: tumor size.

FIGS. 18A-18D include diagrams showing formation of BsAb Armed-NKT cells with various anti-CD3/anti-TAA BsAbs. FIG. 18A: OKT3 (left) and CTA03Fab/CTAT03scFv (right). FIG. 18B: CTA03Fab/CTAT04scFv (left) and CTA03Fab/CTAT05scFv (right). FIG. 18C: OKT3, CTA01scFv/CTAT03Fab, and CTA02scFv/CTAT03Fab (left to right). FIG. 18D: CTA03scFv/CTAT03Fab, CTA04scFv/CTAT03Fab, and CTA05scFv/CTAT03Fab (left to right). NKT cells (CD8⁺ CD25⁺) were cultured and differentiated in the presence of the OKT3 antibody or the BsAbs as indicated. The cells were then were stained with anti-CD8 antibody, anti-CD56 antibody and FITC-conjugated anti-Human IgGFab antibody. BsAbs on cell surface were analyzed using flow cytometry.

FIGS. 19A-19C include charts showing binding activity and toxicity of point-mutant BsAbs CTA03Fab/CTAT02scFv. FIG. 19A: binding activity to CD3+ T cells (Jurkat). FIG. 19B: binding activity to CD19⁺ B cells lymphoma (Raji). FIG. 19C: cytotoxicity of armed T cells relative to T cells cultured with OKT3. The cells were included with CTA03Fab/CTAT02scFv, CTA03-01Fab/CTAT02-01scFv, CTA03-01Fab/CTAT02-02scFv, CTA03-02Fab/CTAT02-02scFv and CTA03-02Fab/CTAT02-01scFv BsAbs, and then stained with FITC conjugated Goat anti-Human IgG Fab antibody. Fluorescent signal on cell surface was detected using flow cytometry. For cytotoxicity analysis, T cells were cultured with OKT3 or with the various BsAbs to form armed T cells. The cells were then cocultured with CD19⁺ B cell lymphoma (Raji) at several effector cell: target cell ratios (3:1, 5:1 and 10:1) for 18 hr. Tumor cell death was determined with CytoTox 96® Non-Radioactive Cytotoxicity Assay (Promega, G1780).

### DETAILED DESCRIPTION OF THE INVENTION

The present disclosure is based on the development of bispecific antibodies (BsAbs) capable of binding to CD3 (e.g., human CD3) and a tumor associated antigen (TAA). Such BsAbs are capable of attaching to the surface of CD3-positive immune cells via binding of the anti-CD3 moiety in the BsAb to the cell surface CD3 to produce armed immune cells. As used herein, the term “an armed immune cell” refers to an immune cell that displays a bispecific antibody as disclosed herein via binding of the anti-CD3 moiety in the bispecific antibody to a cell surface CD3 molecule. Via the anti-TAA moiety in the bispecific antibody on the cell surface, an armed immune cell is capable of targeting disease cells (e.g., cancer cells) that express the TAA, thereby eliciting immune responses against the disease cells.

It is reported herein that the BsAbs disclosed herein show high binding activities to both CD3⁺ immune cells and TAA⁺ cancer cells and high retention levels on CD3⁺ immune cells for at least 72 hours. Immune cells armed with the BsAbs disclosed herein exhibited high cytotoxicity against cancer cells expressing the corresponding TAA both in vitro and in vivo. Thus, the BsAbs and the armed immune cells disclosed herein would be expected to have high anti-cancer effects.

Accordingly, provided herein are bispecific antibodies capable of binding to CD3 and an TAA, armed immune cells displaying such, methods of using the bispecific antibodies for producing armed immune cells, and methods of treating cancer using the armed immune cells.

### I. Bispecific Antibodies That Bind CD3 and a Tumor Associated Antigen

In some aspects, the present disclosure provides bispecific antibodies capable of binding to CD3 (e.g., CD3+ cells) and a tumor associated antigen (TAA) (e.g., cancer cells expressing the TAA on cell surface). An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. A bispecific antibody as disclosed herein comprises two antigen-binding moieties, one of which binds CD3 such as human CD3 and the other one of which binds a tumor associated antigen such as those disclosed herein.

A typical antibody molecule comprises a heavy chain variable region (V_{H}) and a light chain variable region (V_{L}), which are usually involved in antigen binding. The V_{H} and V_{L}. regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FRI, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, an antibody moiety disclosed herein may share the same heavy chain and/or light chain complementary determining regions (CDRs) or the same V_{H} and/or V_{L} chains as a reference antibody. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-CD19 antibodies may have the same V_{H}, the same V_{L}, or both as compared to an exemplary antibody described herein.

In some embodiments, an antibody moiety disclosed herein may share a certain level of sequence identity as compared with a reference sequence. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, an antibody moiety disclosed herein may have one or more amino acid variations relative to a reference antibody. The amino acid residue variations as disclosed in the present disclosure (e.g., in framework regions and/or in CDRs) can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.
A. Bispecific Antibodies

The bispecific antibodies disclosed herein comprise a CD3 binding moiety (anti-CD3 moiety) and a TAA binding moiety (anti-TAA moiety).
(i) CD3 Binding Moieties

The anti-CD3 moiety in any of the bispecific antibodies disclosed herein comprises an antigen-binding fragment specific to a CD3 molecule, for example, human CD3. In some embodiments, the anti-CD3 moiety comprises a heavy chain variable region (V_{H}) and a light chain variable region (V_{L}). In some instances, the anti-CD3 moiety may be derived from a reference anti-CD3 antibody. Exemplary reference anti-CD3 antibodies include CTA.02, CTA.03, CTA.04, or CTA.05. The structural information of these reference anti-CD3 antibodies are provided in Table 1 below (heavy chain and light chain complementary determining regions (CDRs) based on the Kabat scheme are in boldface and underlined).

TABLE 1
Reference anti-CD3 Antibodies

| Reference | | | SEQ ID |
|---|---|---|---|
| Antibody | | Amino Acid Sequences | NO |
| CTA.02 | VH | RYTMH | 1 |
| | CDR1 | | |
| | VH | YINPSRGYINYNQKERD | 2 |
| | CDR2 | | |
| | VH | YYDDHYCEDY | 3 |
| | CDR3 | | |
| | VH | QVQLQQSGAELARPGASVKMSCKASGYTFTRYTMHWVKQRPGQ | 4 |
| | | GLEWIGYINPSRGYTNYNQKFKDKATLTTDKSSSTAYMQLSSL | |
| | | TSEDSAVYYCARYYDDHYCLDYWGQGTTLTVSS | |
| | VL CDR1 | SASSSVSYMN | 5 |
| | VL CDR2 | DTSKLAS | 6 |
| | VL CDR3 | QQWSSNPFT | 7 |
| | VL | QIVLTQSPAIMSASPGEKVTMTCSASSSVSYMNWYQQKSGTSP | 8 |
| | | KRWIYDTSKLASGVPAHFRGSGSGTSYSLTISGMEAEDAATYY | |
| | | CQQWSSNPFTFGSGTKLEINR | |
| | scFv | GGGSGGGQVQLQQSGAELARPGASVKMSCKASGYTFTRYTMHW | 9 |
| | | VKQRPGQGLEWIGYINPSRGYTNYNQKFKDKATLTTDKSSSTA | 250 |
| | | YMQLSSLTSEDSAVYYCARYYDDHYCLDYWGQGTTLTVSSGGG | (no |
| | | GSGGGGSGGGGSQIVLTQSPAIMSASPGEKVTMTCSASSSVSY | N-terminus |
| | | MNWYQQKSGTSPKRWIYDTSKLASGVPAHFRGSGSGTSYSLTI | peptide |
| | | SGMEAEDAATYYCQQWSSNPFTFGSGTKLEINR | linker) |
| | VH-CH1 | QVQLQQSGAELARPGASVKMSCKASGYTFTRYTMHWVKQRPGQ | 10 |
| | | GLEWIGYINPSRGYTNYNQKFKDKATLTTDKSSSTAYMQLSSL | |
| | | TSEDSAVYYCARYYDDHYCLDYWGQGTTLTVSSAKTTAPSVYP | |
| | | LAPVCGGTTGSSVTLGCLVKGYFPEPVTLTWNSGSLSSGVHTF | |
| | | PAVLQSDLYTLSSSVTVTSSTWPSQSITCNVAHPASSTKVDKK | |
| | | I | |
| | VL-Ck | QIVLTQSPAIMSASPGEKVTMTCSASSSVSYMNWYQQKSGTSP | 11 |
| | | KRWIYDTSKLASGVPAHFRGSGSGTSYSLTISGMEAEDAATYY | |
| | | CQQWSSNPFTFGSGTKLEINRADTAPTVSIFPPSSEQLTSGGA | |
| | | SVVCFLNNFYPKDINVKWKIDGSERQNGVLNSWTDQDSKDSTY | |
| | | SMSSTLTLTKDEYERHNSYTCEATHKTSTSPIVKSFNRNEC | |
| CTA.03 | VH | SFPMA | 12 |
| | CDR1 | | |
| | VH | TISTSGGRTYYRDSVKG | 13 |
| | CDR2 | | |
| | VH | FRQYSGGFDY | 14 |
| | CDR3 | | |
| | VH | EVQLLESGGGLVQPGGSLRLSCAASGFTFSSFPMAWVRQAPGK | 15 |
| | | GLEWVSTISTSGGRTYYRDSVKGRFTISRDNSKNTLYLQMNSL | |
| | | RAEDTAVYYCAKFRQYSGGFDYWGQGTLVTVSS | |
| | VL CDR1 | TLSSGNIENNYVH | 16 |
| | VL CDR2 | DDDKRPD | 17 |
| | VL | DDDKRPE | 18 |
| | CDR2-02 | | |
| | VL CDR3 | HSYVSSFNV | 19 |
| | VL | DIQLTQPNSVSTSLGSTVKLSCTLSSGNIENNYVHWYQLYEGR | 20 |
| | | SPTTMIYDDDKRPDGVPDRFSGSIDRSSNSAFLTIHNVAIEDE | |
| | | AIYFCHSYVSSFNVFGGGTKLTVLRQ | |
| | VL-01 | DIQLTQPNSVSTSLGSTVKLSCTLSSGNIENNYVHWYQLYEGR | 21 |
| | | SPTTMIYDDDKRPDAVPDRFSGSIDRSSNSAFLTIHNVAIEDE | |
| | | AIYFCHSYVSSFNVFGGGTKLTVLRQ | |
| | VL-02 | DIQLTQPNSVSTSLGSTVKLSCTLSSGNIENNYVHWYQLYEGR | 249 |
| | | SPTTMIYDDDKRPEGVPDRFSGSIDRSSNSAFLTIHNVAIEDE | |
| | | AIYFCHSYVSSFNVFGGGTKLTVLRQ | |
| | scFv | GGGSGGGEVQLLESGGGLVQPGGSLRLSCAASGFTFSSFPMAW | 22 |
| | | VRQAPGKGLEWVSTISTSGGRTYYRDSVKGRFTISRDNSKNTL | 251 |
| | | YLQMNSLRAEDTAVYYCAKFRQYSGGFDYWGQGTLVTVSSGGG | (no |
| | | GSGGGGSGGGGSDIQLTQPNSVSTSLGSTVKLSCTLSSGNIEN | N-terminus |
| | | NYVHWYQLYEGRSPTTMIYDDDKRPDGVPDRFSGSIDRSSNSA | peptide |
| | | FLTIHNVAIEDEAIYFCHSYVSSFNVFGGGTKLTVLRQ | linker) |
| | VH-CH1 | EVQLLESGGGLVQPGGSLRLSCAASGFTFSSFPMAWVRQAPGK | 23 |
| | | GLEWVSTISTSGGRTYYRDSVKGRFTISRDNSKNTLYLQMNSL | |
| | | RAEDTAVYYCAKFRQYSGGFDYWGQGTLVTVSSASTKGPSVFP | |
| | | LAPSSKSTSGGTAALGCLVKDYFPEPVTVSWNSGALTSGVHTF | |
| | | PAVLQSSGLYSLSSVVTVPSSSLGTQTYICNVNHKPSNTKVDK | |
| | | KVEPKSC | |
| | VL-Cλ | DIQLTQPNSVSTSLGSTVKLSCTLSSGNIENNYVHWYQLYEGR | 24 |
| | | SPTTMIYDDDKRPDGVPDRFSGSIDRSSNSAFLTIHNVAIEDE | |
| | | AIYFCHSYVSSFNVFGGGTKLTVLRQPKAAPSVTLFPPSSEEL | |
| | | QANKATLVCLISDFYPGAVTVAWKADSSPVKAGVETTTPSKQS | |
| | | NNKYAASSYLSLTPEQWKSHRSYSCQVTHEGSTVEKTVAPTEC | |
| | | S | |
| | VL-Cλ-01 | DIQLTQPNSVSTSLGSTVKLSCTLSSGNIENNYVHWYQLYEGR | 228 |
| | | SPTTMIYDDDKRPDAVPDRFSGSIDRSSNSAFLTIHNVAIEDE | |
| | | AIYFCHSYVSSFNVFGGGTKLTVLRQPKAAPSVTLFPPSSEEL | |
| | | QANKATLVCLISDFYPGAVTVAWKADSSPVKAGVETTTPSKQS | |
| | | NNKYAASSYLSLTPEQWKSHRSYSCQVTHEGSTVEKTVAPTEC | |
| | | S | |
| | VL-Cλ-02 | DIQLTQPNSVSTSLGSTVKLSCTLSSGNIENNYVHWYQLYEGR | 25 |
| | | SPTTMIYDDDKRPEGVPDRFSGSIDRSSNSAFLTIHNVAIEDE | |
| | | AIYFCHSYVSSFNVFGGGTKLTVLRQPKAAPSVTLFPPSSEEL | |
| | | QANKATLVCLISDFYPGAVTVAWKADSSPVKAGVETTTPSKQS | |
| | | NNKYAASSYLSLTPEQWKSHRSYSCQVTHEGSTVEKTVAPTEC | |
| | | S | |
| CTA.04 | VH | RYTMH | 26 |
| | CDR1 | | |
| | VH | YINPSRGYTNYNQKVKD | 27 |
| | CDR2 | | |
| | VH | YYDDHYCLDY | 28 |
| | CDR3 | | |
| | VH | QVQLVQSGGGVVQPGRSLRLSCKASGYTFTRYTMHWVRQAPGK | 29 |
| | | GLEWIGYINPSRGYTNYNQKVKDRFTISRDNSKNTAFLQMDSL | |
| | | RPEDTGVYFCARYYDDHYCLDYWGQGTPVTVSS | |
| | VL CDR1 | SASSSVSYMN | 30 |
| | VL CDR2 | DTSKLAS | 31 |
| | VL CDR3 | QQWSSNPFT | 32 |
| | VL | DIQMTQSPSSLSASVGDRVTITCSASSSVSYMNWYQQTPGKAP | 33 |
| | | KRWIYDTSKLASGVPSRFSGSGSGTDYTFTISSLQPEDIATYY | |
| | | CQQWSSNPFTFGQGTKLQITR | |
| | scFv | GGGSGGGQVQLVQSGGGVVQPGRSLRLSCKASGYTFTRYTMHW | 34 |
| | | VRQAPGKGLEWIGYINPSRGYTNYNQKVKDRFTISRDNSKNTA | 252 |
| | | FLQMDSLRPEDIGVYFCARYYDDHYCLDYWGQGTPVTVSSGGG | (no |
| | | GSGGGGSGGGGSDIQMTQSPSSLSASVGDRVTITCSASSSVSY | N-terminus |
| | | MNWYQQTPGKAPKRWIYDTSKLASGVPSRFSGSGSGTDYTFTI | peptide |
| | | SSLQPEDIATYYCQQWSSNPFTFGQGTKLQITR | linker) |
| | VH-CH1 | QVQLVQSGGGVVQPGRSLRLSCKASGYTFTRYTMHWVRQAPGK | 35 |
| | | GLEWIGYINPSRGYTNYNQKVKDRFTISRDNSKNTAFLQMDSL | |
| | | RPEDTGVYFCARYYDDHYCLDYWGQGTPVTVSSASTKGPSVFP | |
| | | LAPSSKSTSGGTAALGCLVKDYFPEPVTVSWNSGALTSGVHTF | |
| | | PAVLQSSGLYSLSSVVTVPSSSLGTQTYICNVNHKPSNTKVDK | |
| | | KVEPKSC | |
| | VL-Ck | DIQMTQSPSSLSASVGDRVTITCSASSSVSYMNWYQQTPGKAP | 36 |
| | | KRWIYDTSKLASGVPSRFSGSGSGTDYTFTISSLQPEDIATYY | |
| | | CQQWSSNPFTFGQGTKLQITRTVAAPSVFIFPPSDEQLKSGTA | |
| | | SVVCLLNNFYPREAKVQWKVDNALQSGNSQESVTEQDSKDSTY | |
| | | SLSSTLTLSKADYEKHKVYACEVTHQGLSSPVTKSENRGEC | |
| CTA.05 | VH | SYTMH | 37 |
| | CDR1 | | |
| | VH | YINPRSGYTHYNQKLKD | 38 |
| | CDR2 | | |
| | VH | SAYYDYDGFAY | 39 |
| | CDR3 | | |
| | VH | QVQLVQSGAEVKKPGASVKVSCKASGYTFISYTMHWVRQAPGQ | 40 |
| | | GLEWMGYINPRSGYTHYNQKLKDKATLTADKSASTAYMELSSL | |
| | | RSEDTAVYYCARSAYYDYDGFAYWGQGTLVTVSS | |
| | VL CDR1 | SASSSVSYMN | 41 |
| | VL CDR2 | DTSKLAS | 42 |
| | VL CDR3 | QQWSSNPPT | 43 |
| | VL | DIQMTQSPSSLSASVGDRVTITCSASSSVSYMNWYQQKPGKAP | 44 |
| | | KRLIYDTSKLASGVPSRFSGSGSGTDFTLTISSLQPEDFATYY | |
| | | CQQWSSNPPTFGGGTKVEIK | |
| | scFv | GGGSGGGQVQLVQSGAEVKKPGASVKVSCKASGYTFISYTMHW | 45 |
| | | VRQAPGQGLEWMGYINPRSGYTHYNQKLKDKATLTADKSASTA | 253 |
| | | YMELSSLRSEDTAVYYCARSAYYDYDGFAYWGQGTLVTVSSGG | (no |
| | | GGSGGGGSGGGGSDIQMTQSPSSLSASVGDRVTITCSASSSVS | N-terminus |
| | | YMNWYQQKPGKAPKRLIYDTSKLASGVPSRFSGSGSGTDFTLT | peptide |
| | | ISSLQPEDFATYYCQQWSSNPPTFGGGTKVEIK | linker) |
| | VH-CH1 | QVQLVQSGAEVKKPGASVKVSCKASGYTFISYTMHWVRQAPGQ | 46 |
| | | GLEWMGYINPRSGYTHYNQKLKDKATLTADKSASTAYMELSSL | |
| | | RSEDTAVYYCARSAYYDYDGFAYWGQGTLVTVSSASTKGPSVF | |
| | | PLAPCSRSTSESTAALGCLVKDYFPEPVTVSWNSGALTSGVHT | |
| | | FPAVLQSSGLYSLSSVAIVTVPSSNFGTQTYTCNVDHKPSNTK | |
| | | VDKTV | |
| | VL-Ck | DIQMTQSPSSLSASVGDRVTITCSASSSVSYMNWYQQKPGKAP | 47 |
| | | KRLIYDTSKLASGVPSRFSGSGSGTDFTLTISSLQPEDFATYY | |
| | | CQQWSSNPPTFGGGTKVEIKRTVAAPSVFIFPPSDEQLKSGTA | |
| | | SVVCLLNNFYPREAKVQWKVDNALQSGNSQESVTEQDSKDSTY | |
| | | SLSSTLTLSKADYEKHKLYACEVTHQGLSSPVTKSENRGEC | |

An anti-CD3 binding moiety (and an anti-TAA binding moiety disclosed below) derived from a reference antibody refers to binding moieties having substantially similar structural and functional features as the reference antibody. Structurally, the binding moiety may have the same heavy and/or light chain complementary determining regions or the same V_{H} and/or V_{L} chains as the reference antibody. Alternatively, the binding moiety may only have a limited number of amino acid variations in one or more of the framework regions and/or in one or more of the CDRs without significantly affecting its binding affinity and binding specificity relative to the reference antibody.

In some embodiments, the anti-CD3 binding moiety may comprise the same heavy chain CDRs as those in antibody CTA.02, which are provided in Table 1 above. Alternatively or in addition, the anti-CD3 binding moiety may have the same light chain CDRs as those in antibody CTA.02, which are also provided in Table 1 above. Such an anti-CD3 binding moiety may comprise the same V_{H} and/or V_{L} chains as CTA.02. Alternatively, the anti-CD3 binding moiety may comprise amino acid variations in one or more of the framework regions relative to the corresponding framework regions in CTA.02. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.02.

In some embodiments, the anti-CD3 moiety may comprise a certain level of variations in one or more of the CDRs relative to those of CTA.02. For example, the anti-CD3 moiety may comprise heavy chain CDRs that are at least 80% (e.g., 85%, 90%, 95%, or 98%) sequence identity, individually or collectively, as compared with the V_{H} CDRs of CTA.02. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.02. As used herein, “individually” means that one CDR of an antibody shares the indicated sequence identity relative to the corresponding CDR of a reference antibody (e.g., the anti-CD3 reference antibodies provided in Table 1 above or any of the anti-TAA reference antibodies disclosed below). “Collectively” means that three V_{H} or V_{L} CDRs of an antibody in combination share the indicated sequence identity relative the corresponding three V_{H} or V_{L} CDRs of the reference antibody in combination.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.02. In some instances, the anti-CD3 moiety may comprise the same heavy chain CDR3 as the heavy chain CDR3 of CTA.02 and comprise one or more amino acid variations in one or more of the other heavy chain and light chain CDRs.

In some embodiments, the anti-CD3 binding moiety may comprise the same heavy chain CDRs as those in antibody CTA.03, which are provided in Table 1 above. Alternatively or in addition, the anti-CD3 binding moiety may have the same light chain CDRs as those in antibody CTA.03, which are also provided in Table 1 above. Such an anti-CD3 binding moiety may comprise the same V_{H} and/or V_{L} chains as CTA.03. Alternatively, the anti-CD3 binding moiety may comprise amino acid variations in one or more of the framework regions relative to the corresponding framework regions in CTA.03. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.03. In one specific example, the anti-CD3 moiety disclosed herein comprises a mutation at position G58 of the V_{L} chain relative to CTA.03, for example, an amino acid residue substitution (e.g., G58A). See, e.g., CTA.03 VL-01 in Table 1 above.

In some embodiments, the anti-CD3 moiety may comprise a certain level of variations in one or more of the CDRs relative to those of CTA.03. For example, the anti-CD3 moiety may comprise heavy chain CDRs that are at least 80% (e.g., 85%, 90%, 95%, or 98%) sequence identity, individually or collectively, as compared with the V_{H} CDRs of CTA.03. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.03.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.03. In some instances, the anti-CD3 moiety may comprise the same heavy chain CDR3 as the heavy chain CDR3 of CTA.03 and comprise one or more amino acid variations in one or more of the other heavy chain and light chain CDRs. In specific examples, the anti-CD3 moiety disclosed herein may comprise a mutation at position D57 of the V_{L} chain relative to that of CTA.03, for example, an amino acid residue substitution such as D57E. See, e.g., CTA.03 VL-02 in Table 1.

In some examples, the anti-CD3 binding moiety may comprise the same heavy chain CDRs as those in antibody CTA.04, which are provided in Table 1 above. Alternatively or in addition, the anti-CD3 binding moiety may have the same light chain CDRs as those in antibody CTA.04, which are also provided in Table 1 above. Such an anti-CD3 binding moiety may comprise the same V_{H} and/or V_{L} chains as CTA.04. Alternatively, the anti-CD3 binding moiety may comprise amino acid variations in one or more of the framework regions relative to the corresponding framework regions in CTA.04. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.04.

In some embodiments, the anti-CD3 moiety may comprise a certain level of variations in one or more of the CDRs relative to those of CTA.04. For example, the anti-CD3 moiety may comprise heavy chain CDRs that are at least 80% (e.g., 85%, 90%, 95%, or 98%) sequence identity, individually or collectively, as compared with the V_{H} CDRs of CTA.04. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.04.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.04. In some instances, the anti-CD3 moiety may comprise the same heavy chain CDR3 as the heavy chain CDR3 of CTA.04 and comprise one or more amino acid variations in one or more of the other heavy chain and light chain CDRs.

In some examples, the anti-CD3 binding moiety may comprise the same heavy chain CDRs as those in antibody CTA.05, which are provided in Table 1 above. Alternatively or in addition, the anti-CD3 binding moiety may have the same light chain CDRs as those in antibody CTA.05, which are also provided in Table 1 above. Such an anti-CD3 binding moiety may comprise the same V_{H} and/or V_{L} chains as CTA.05. Alternatively, the anti-CD3 binding moiety may comprise amino acid variations in one or more of the framework regions relative to the corresponding framework regions in CTA.05. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.05.

In some embodiments, the anti-CD3 moiety may comprise a certain level of variations in one or more of the CDRs relative to those of CTA.05. For example, the anti-CD3 moiety may comprise heavy chain CDRs that are at least 80% (e.g., 85%, 90%, 95%, or 98%) sequence identity, individually or collectively, as compared with the V_{H} CDRs of CTA.05. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.05.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.05. In some instances, the anti-CD3 moiety may comprise the same heavy chain CDR3 as the heavy chain CDR3 of CTA.05 and comprise one or more amino acid variations in one or more of the other heavy chain and light chain CDRs.
(ii) TAA Binding Moiety

In addition to the anti-CD3 binding moiety, any of the bispecific antibodies disclosed herein further comprises a second binding moiety specific to a tumor associated antigen. The term “tumor-associated antigen” (TAA) is well-understood in the art, and refers to a molecule that is differentially expressed on and/in cancerous cells relative to non-cancerous cells of the same cell type. Non-limiting examples of TAA include CD5, CD19, CD20, CD22, CD23, CD25, CD27, CD30, CD33, CD34, CD37, CD38, CD40, CD43, CD44v6, CD47, CD50, CD52, CD56, CD63, CD72a, CD74, CD78, CD79a, CD79b, CD86, CD134, CD137, CD138, CD248, CD319, αvβ3, α5β1, human epidermal growth factor receptor (EGFR or HER1), HER2, HER3, HER4, vascular endothelial growth factor receptor 1 (VEGFR-1), VEGFR-2, VEGFR-3, TRAIL-R2, carbohydrate antigen 19-9 (CA 19-9), carbohydrate antigen 125 (CA 125), carcinoembryonic antigen (CEA), mucin 1 (MUC 1), MUC2, MUC3, MUC4, MUC5, MUC7, ganglioside GD2, ganglioside GD3, ganglioside GM2, carbonic anhydrase IX (CAIX), sonic hedgehog (SHH), melanoma chondroitin sulfate proteoglycan (MCSP), chondroitin sulfate proteoglycan 4 (CSPG4), six-transmembrane epithelial antigen of prostate (STEAP), A33 antigen, desmoglein-2 (Dsg2), Dsg3, Dsg4, E-cadherin neoepitope, fetal nicotinic acetylcholine receptor (fnAChR), muellerian inhibitory substance receptor type II (MISIIR), tumor-associated antigen L6 (TAL6), Thomsen-Friedenreich (TF) antigen, EPHA1, EPHA2, EPHA3, EPHA4, EPHA7, EPHA8, EPHA10, EPHB4, cancer testis antigen (CTA), NY-BRI, tumor-associated glycoprotein 72 (TAG-72), alpha-fetoprotein (AFP), brother of the regulator of the imprinted site (BORIS), B-cell activating factor (BAFF), extradomain-B fibronectin (EDB-FN), glycoprotein A33 (GPA33), tenascin-C (TNC), melanoma-associated antigen (MAGE), GAGE, BAGE, prostate stem cell antigen (PSCA), mesothelin, mucine-related Tn, Sialyl Tn, globo H, stage-specific embryonic antigen-4 (SSEA-4), epithelial cell adhesion molecule (EpCAM), cytotoxic T-lymphocyte-associated protein 4 (CTLA-4), programmed cell death 1 (PD-1), programmed cell death 1 ligand 1 (PD-L1), prostate-specific membrane antigen (PSMA), fibroblast activation protein (FAP), vascular cell adhesion protein 1 (VCAM-1), insulin-like growth factor receptor (IGFR), or hepatocyte growth factor receptor (HGFR).

In some embodiments, the anti-TAA binding moiety comprises a heavy chain variable region (V_{H}) and a light chain variable region (V_{L}). In some examples, the anti-TAA binding moiety is specific to CD20 (e.g., human CD20). In some examples, the anti-TAA binding moiety is specific to CD19 (e.g., human CD19). In some examples, the anti-TAA binding moiety is specific to EGFR (e.g., human EGFR). In some examples, the anti-TAA binding moiety is specific to HER2 (e.g., human HER2). In some examples, the anti-TAA binding moiety is specific to PSMA (e.g., human PSMA). In some examples, the anti-TAA binding moiety is specific to CEA (e.g., human CEA). In some examples, the anti-TAA binding moiety is specific to EpCAM (e.g., human EpCAM). In some examples, the anti-TAA binding moiety is specific to FAP (e.g., human FAP). In some examples, the anti-TAA binding moiety is specific to PDL1 (e.g., human PDL1). In some examples, the anti-TAA binding moiety is specific to CD38 (e.g., human CD38). In some examples, the anti-TAA binding moiety is specific to CD33 (e.g., human CD33). In some examples, the anti-TAA binding moiety is specific to HGFR (cMET) (e.g., human cMET). In some examples, the anti-TAA binding moiety is specific to CD47 (e.g., human CD47). In some examples, the anti-TAA binding moiety is specific to TRAIL-R2 (e.g., human TRAIL-R2). In some examples, the anti-TAA binding moiety is specific to mesothelin (e.g., human mesothelin). In some examples, the anti-TAA binding moiety is specific to GD2 (e.g., human GD2).

In some instances, the anti-TAA moiety may be derived from a reference anti-TAA antibody. Exemplary reference anti-TAA antibodies include CTAT.01-CTAT.16. The structural information of these reference anti-CD3 antibodies are provided in Table 2 below (heavy chain and light chain complementary determining regions (CDRs) based on the Kabat scheme are in boldface and underlined).

TABLE 2
Reference Anti-Tumor Associated Antigen Antibodies

| Reference | | | SEQ ID |
|---|---|---|---|
| Antibody (TAA) | | Amino Acid Sequences | NO |
| CTAT.01 | VH | SYNMH | 48 |
| (CD20) | CDR1 | | |
| | VH | AIYPGNGDISYNQKEKG | 49 |
| | CDR2 | | |
| | VH | STYYGGDWYFNV | 50 |
| | CDR3 | | |
| | VH | QVQLQQPGAELVKPGASVKMSCKASGYTFTSYNMHWVKQTPGRGLEW | 51 |
| | | IGAIYPGNGDTSYNQKFKGKATLTADKSSSTAYMQLSSLTSEDSAVY | |
| | | YCARSTYYGGDWYFNVWGAGTTVTVSA | |
| | VL | RASSSVSYIH | 52 |
| | CDR1 | | |
| | VL | ATSNLAS | 53 |
| | CDR2 | | |
| | VL | QQWTSNPPT | 54 |
| | CDR3 | | |
| | VL | QIVLSQSPAILSASPGEKVTMTCRASSSVSYIHWFQQKPGSSPKPWI | 55 |
| | | YATSNLASGVPVRFSGSGSGTSYSLTISRVEAEDAATYYCQQWTSNP | |
| | | PTFGGGTKLEIK | |
| | scFv | GGGSGGGQVQLQQPGAELVKPGASVKMSCKASGYTFTSYNMHWVKQT | 56 |
| | | PGRGLEWIGAIYPGNGDTSYNQKFKGKATLTADKSSSTAYMQLSSLT | 254 |
| | | SEDSAVYYCARSTYYGGDWYFNVWGAGTTVTVSAGGGGSGGGGSGGG | (no |
| | | GSQIVLSQSPAILSASPGEKVTMTCRASSSVSYIHWFQQKPGSSPKP | N-terminus |
| | | WIYATSNLASGVPVRFSGSGSGTSYSLTISRVEAEDAATYYCQQWTS | peptide |
| | | NPPTFGGGTKLEIK | linker) |
| | VH-CH1 | QVQLQQPGAELVKPGASVKMSCKASGYTFTSYNMHWVKQTPGRGLEW | 57 |
| | | IGAIYPGNGDTSYNQKFKGKATLTADKSSSTAYMQLSSLTSEDSAVY | |
| | | YCARSTYYGGDWYFNVWGAGTTVTVSAASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKAEPKSC | |
| | VL-Ck | QIVLSQSPAILSASPGEKVTMTCRASSSVSYIHWFQQKPGSSPKPWI | 58 |
| | | YATSNLASGVPVRFSGSGSGTSYSLTISRVEAEDAATYYCQQWTSNP | |
| | | PTFGGGTKLEIKRTVAAPSVFIFPPSDEQLKSGTASVVCLLNNFYPR | |
| | | EAKVQWKVDNALQSGNSQESVTEQDSKDSTYSLSSTLTLSKADYEKH | |
| | | KVYACEVTHQGLSSPVTKSENRGEC | |
| CTAT.02 | VH | DYGVS | 59 |
| (CD19) | CDR1 | | |
| | VH | VIWGSETTYYNSALKS | 60 |
| | CDR2 | | |
| | VH | HYYYGGSYAMDY | 61 |
| | CDR3 | | |
| | VH | EVKLQESGPGLVAPSQSLSVTCTVSGVSLPDYGVSWIRQPPRKGLEW | 62 |
| | | LGVIWGSETTYYNSALKSRLTIIKDNSKSQVFLKMNSLQTDDTAIYY | |
| | | CAKHYYYGGSYAMDYWGQGTSVTVSS | |
| | VL | RASQDISKYLN | 63 |
| | CDR1 | | |
| | VL | HTSRLHS | 64 |
| | CDR2 | | |
| | VL | QQGNTLPYT | 65 |
| | CDR3 | | |
| | VL | DIQMTQTTSSLSASLGDRVTISCRASQDISKYLNWYQQKPDGTVKLL | 66 |
| | | IYHTSRLHSGVPSRFSGSGSGTDYSLTISNLEQEDIATYFCQQGNTL | |
| | | PYTFGGGTKLEIT | |
| | VL-01 | DIQMTQTTSSLSASLGDRVTISCRASQDISKYLNWYQQKPDATVKLL | 67 |
| | | IYHTSRLHSGVPSRFSGSGSGTDYSLTISNLEQEDIATYFCQQGNTL | |
| | | PYTFGGGTKLEIT | |
| | VL-02 | DIQMTQTTSSLSASLGDRVTISCRASQDISKYLNWYQQKPEGTVKLL | 68 |
| | | IYHTSRLHSGVPSRFSGSGSGTDYSLTISNLEQEDIATYFCQQGNTL | |
| | | PYTFGGGTKLEIT | |
| | scFv | GGGSGGGEVKLQESGPGLVAPSQSLSVTCTVSGVSLPDYGVSWIRQP | 69 |
| | | PRKGLEWLGVIWGSETTYYNSALKSRLTIIKDNSKSQVFLKMNSLQT | 255 |
| | | DDTAIYYCAKHYYYGGSYAMDYWGQGTSVTVSSGGGGSGGGGSGGGG | (no |
| | | SDIQMTQTTSSLSASLGDRVTISCRASQDISKYLNWYQQKPDGTVKL | N-terminus |
| | | LIYHTSRLHSGVPSRFSGSGSGTDYSLTISNLEQEDIATYFCQQGNT | peptide |
| | | LPYTFGGGTKLEIT | linker) |
| | scFv-01 | GGGSGGGEVKLQESGPGLVAPSQSLSVTCTVSGVSLPDYGVSWIRQP | 70 |
| | | PRKGLEWLGVIWGSETTYYNSALKSRLTIIKDNSKSQVELKMNSLQT | 256 |
| | | DDTAIYYCAKHYYYGGSYAMDYWGQGTSVTVSSGGGGSGGGGSGGGG | (no |
| | | SDIQMTQTTSSLSASLGDRVTISCRASQDISKYLNWYQQKPDATVKL | N-terminus |
| | | LIYHTSRLHSGVPSRFSGSGSGTDYSLTISNLEQEDIATYFCQQGNT | peptide |
| | | LPYTFGGGTKLEIT | linker) |
| | scFv-02 | GGGSGGGEVKLQESGPGLVAPSQSLSVTCTVSGVSLPDYGVSWIRQP | 71 |
| | | PRKGLEWLGVIWGSETTYYNSALKSRLTIIKDNSKSQVFLKMNSLQT | 257 |
| | | DDTAIYYCAKHYYYGGSYAMDYWGQGTSVTVSSGGGGSGGGGSGGGG | (no |
| | | SDIQMTQTTSSLSASLGDRVTISCRASQDISKYLNWYQQKPEGTVKL | N-terminus |
| | | LIYHTSRLHSGVPSRFSGSGSGTDYSLTISNLEQEDIATYFCQQGNT | peptide |
| | | LPYTFGGGTKLEIT | linker) |
| | VH-CH1 | EVKLQESGPGLVAPSQSLSVTCTVSGVSLPDYGVSWIRQPPRKGLEW | 72 |
| | | LGVIWGSETTYYNSALKSRLTIIKDNSKSQVFLKMNSLQTDDTAIYY | |
| | | CAKHYYYGGSYAMDYWGQGTSVTVSSASTKGPSVFPLAPSSKSTSGG | |
| | | TAALGCLVKDYFPEPVTVSWNSGALTSGVHTFPAVLQSSGLYSLSSV | |
| | | VTVPSSSLGTQTYICNVNHKPSNTKVDKRVEPKSCDK | |
| | VL-Ck | DIQMTQTTSSLSASLGDRVTISCRASQDISKYLNWYQQKPDGTVKLL | 73 |
| | | IYHTSRLHSGVPSRFSGSGSGTDYSLTISNLEQEDIATYFCQQGNTL | |
| | | PYTFGGGTKLEITRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSENRGEC | |
| CTAT.03 | VH | NYGVH | 74 |
| (EGFR) | CDR1 | | |
| | VH | VIWSGGNTDYNTPFTS | 75 |
| | CDR2 | | |
| | VH | ALTYYDYEFAY | 76 |
| | CDR3 | | |
| | VH | QVQLKQSGPGLVQPSQSLSITCTVSGFSLTNYGVHWVRQSPGKGLEW | 77 |
| | | LGVIWSGGNTDYNTPFTSRLSINKDNSKSQVFFKMNSLQSNDTAIYY | |
| | | CARALTYYDYEFAYWGQGTLVTVSA | |
| | VL | RASQSIGTNIH | 78 |
| | CDR1 | | |
| | VL | YASESIS | 79 |
| | CDR2 | | |
| | VL | QQNNNWPTT | 80 |
| | CDR3 | | |
| | VL | DILLTQSPVILSVSPGERVSFSCRASQSIGTNIHWYQQRINGSPRLL | 81 |
| | | IKYASESISGIPSRFSGSGSGTDFTLSINSVESEDIADYYCQQNNNW | |
| | | PTTFGAGTKLELK | |
| | scFv | GGGSGGGQVQLKQSGPGLVQPSQSLSITCTVSGFSLTNYGVHWVRQS | 82 |
| | | PGKGLEWLGVIWSGGNTDYNTPFTSRLSINKDNSKSQVFFKMNSLQS | 258 |
| | | NDTAIYYCARALTYYDYEFAYWGQGTLVTVSAGGGGSGGGGSGGGGS | (no N-terminus |
| | | DILLTQSPVILSVSPGERVSFSCRASQSIGTNIHWYQQRINGSPRLL | peptide |
| | | IKYASESISGIPSRFSGSGSGTDFTLSINSVESEDIADYYCQQNNNW | linker) |
| | | PTTFGAGTKLELK | |
| | VH-CH1 | QVQLKQSGPGLVQPSQSLSITCTVSGFSLTNYGVHWVRQSPGKGLEW | 83 |
| | | LGVIWSGGNTDYNTPFTSRLSINKDNSKSQVFFKMNSLQSNDTAIYY | |
| | | CARALTYYDYEFAYWGQGTLVTVSAASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVDKRVEPKSCDK | |
| | VL-Ck | DILLTQSPVILSVSPGERVSFSCRASQSIGTNIHWYQQRINGSPRLL | 84 |
| | | IKYASESISGIPSRFSGSGSGTDFTLSINSVESEDIADYYCQQNNNW | |
| | | PTTFGAGTKLELKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSENRGEC | |
| CTAT.04 | VH | DTYIH | 85 |
| (HER2) | CDR1 | | |
| | VH | RIYPTNGYTRYADSVKG | 86 |
| | CDR2 | | |
| | VH | WGGDGFYAMDY | 87 |
| | CDR3 | | |
| | VH | EVQLVESGGGLVQPGGSLRLSCAASGFNIKDTYIHWVRQAPGKGLEW | 88 |
| | | VARIYPTNGYTRYADSVKGRFTISADTSKNTAYLQMNSLRAEDTAVY | |
| | | YCSRWGGDGFYAMDYWGQGTLVTVSS | |
| | VL | RASQDVNTAVA | 89 |
| | CDR1 | | |
| | VL | SASFLYS | 90 |
| | CDR2 | | |
| | VL | QQHYTTPPT | 91 |
| | CDR3 | | |
| | VL | DIQMTQSPSSLSASVGDRVTITCRASQDVNTAVAWYQQKPGKAPKLL | 92 |
| | | IYSASFLYSGVPSRFSGSRSGTDFTLTISSLQPEDFATYYCQQHYTT | |
| | | PPTFGQGTKVEIK | |
| | scFv | GGGSGGGEVQLVESGGGLVQPGGSLRLSCAASGFNIKDTYIHWVRQA | 93 |
| | | PGKGLEWVARIYPTNGYTRYADSVKGRFTISADTSKNTAYLQMNSLR | 259 |
| | | AEDTAVYYCSRWGGDGFYAMDYWGQGTLVTVSSGGGGSGGGGSGGGG | (no N-terminus |
| | | SDIQMTQSPSSLSASVGDRVTITCRASQDVNTAVAWYQQKPGKAPKL | peptide |
| | | LIYSASFLYSGVPSRFSGSRSGTDFTLTISSLQPEDFATYYCQQHYT | linker) |
| | | TPPTFGQGTKVEIK | |
| | VH-CH1 | EVQLVESGGGLVQPGGSLRLSCAASGFNIKDTYIHWVRQAPGKGLEW | 94 |
| | | VARIYPTNGYTRYADSVKGRFTISADTSKNTAYLQMNSLRAEDTAVY | |
| | | YCSRWGGDGFYAMDYWGQGTLVTVSSASTKGPSVFPLAPSSKSTSGG | |
| | | TAALGCLVKDYFPEPVTVSWNSGALTSGVHTFPAVLQSSGLYSLSSV | |
| | | VTVPSSSLGTQTYICNVNHKPSNTKVDKRVEPKSCDK | |
| | VL-Ck | DIQMTQSPSSLSASVGDRVTITCRASQDVNTAVAWYQQKPGKAPKLL | 95 |
| | | IYSASFLYSGVPSRFSGSRSGTDFTLTISSLQPEDFATYYCQQHYTT | |
| | | PPTFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSFNRGEC | |
| CTAT.05 | VH | EYTIH | 96 |
| (PSMA) | CDR1 | | |
| | VH | NINPNNGGTTYNQKFED | 97 |
| | CDR2 | | |
| | VH | GWNFDY | 98 |
| | CDR3 | | |
| | VH | EVQLVQSGAEVKKPGASVKISCKTSGYTFTEYTIHWVKQASGKGLEW | 99 |
| | | IGNINPNNGGTTYNQKFEDRATLTVDKSTSTAYMELSSLRSEDTAVY | |
| | | YCAAGWNFDYWGQGTTVTVSS | |
| | VL | KASQDVGTAVD | 100 |
| | CDR1 | | |
| | VL | WASTRHT | 101 |
| | CDR2 | | |
| | VL | QQYNSYPLT | 102 |
| | CDR3 | | |
| | VL | DIVMTQSPSSLSASVGDRVTITCKASQDVGTAVDWYQQKPGKAPKLL | 103 |
| | | IYWASTRHTGVPDRFTGSGSGTDFTLTISSLQPEDFADYFCQQYNSY | |
| | | PLTFGGGTKLEIK | |
| | scFv | GGGSGGGEVQLVQSGAEVKKPGASVKISCKTSGYTFTEYTIHWVKQA | 104 |
| | | SGKGLEWIGNINPNNGGTTYNQKFEDRATLTVDKSTSTAYMELSSLR | 260 |
| | | SEDTAVYYCAAGWNFDYWGQGTTVTVSSGGGGSGGGGSGGGGSDIVM | (no N-terminus |
| | | TQSPSSLSASVGDRVTITCKASQDVGTAVDWYQQKPGKAPKLLIYWA | peptide |
| | | STRHTGVPDRFTGSGSGTDFTLTISSLQPEDFADYFCQQYNSYPLTF | linker) |
| | | GGGTKLEIK | |
| | VH-CH1 | EVQLVQSGAEVKKPGASVKISCKTSGYTFTEYTIHWVKQASGKGLEW | 105 |
| | | IGNINPNNGGTTYNQKFEDRATLTVDKSTSTAYMELSSLRSEDTAVY | |
| | | YCAAGWNFDYWGQGTTVTVSSASTKGPSVFPLAPSSKSTSGGTAALG | |
| | | CLVKDYFPEPVTVSWNSGALTSGVHTFPAVLQSSGLYSLSSVVTVPS | |
| | | SSLGTQTYICNVNHKPSNTKVDKRVEPKSCDK | |
| | VL-Ck | DIVMTQSPSSLSASVGDRVTITCKASQDVGTAVDWYQQKPGKAPKLL | 106 |
| | | IYWASTRHTGVPDRFTGSGSGTDFTLTISSLQPEDFADYFCQQYNSY | |
| | | PLTFGGGTKLEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSENRGEC | |
| CTAT.06 | VH | EYGMN | 107 |
| (CEA) | CDR1 | | |
| | VH | WINTKSGEATYVEEFKG | 108 |
| | CDR2 | | |
| | VH | WDFYDYVDEAMY | 109 |
| | CDR3 | | |
| | VH | QVQLVQSGSELKKPGASVKVSCKASGYTFTEYGMNVWRQAPGQGLEW | 110 |
| | | MGWINTKSGEATYVEEFKGRFVFSLDTSVSTAYLQISSLKAEDTAVY | |
| | | YCARWDFYDYVDEAMYWGQGTTVTVSS | |
| | VL | KASQTVSANVA | 111 |
| | CDR1 | | |
| | VL | LASYRYR | 112 |
| | CDR2 | | |
| | VL | HQYYTYPLFT | 113 |
| | CDR3 | | |
| | VL | DIQMTQSPSSLSASVGDRVTITCKASQTVSANVAWYQQKPGKAPKLL | 114 |
| | | IYLASYRYRGVPSRFSGSGSGTDFTLTISSLQPEDFATYYCHQYYTY | |
| | | PLFTFGQGTKLEIK | |
| | scFv | GGGSGGGQVQLVQSGSELKKPGASVKVSCKASGYTFTEYGMNVWRQA | 115 |
| | | PGQGLEWMGWINTKSGEATYVEEFKGRFVFSLDTSVSTAYLQISSLK | 261 (no |
| | | AEDTAVYYCARWDFYDYVDEAMYWGQGTTVTVSSGGGGSGGGGSGGG | N-terminus |
| | | GSDIQMTQSPSSLSASVGDRVTITCKASQTVSANVAWYQQKPGKAPK | peptide |
| | | LLIYLASYRYRGVPSRFSGSGSGTDFTLTISSLQPEDFATYYCHQYY | linker) |
| | | TYPLFTFGQGTKLEIK | |
| | VH-CH1 | QVQLVQSGSELKKPGASVKVSCKASGYTFTEYGMNVWRQAPGQGLEW | 116 |
| | | MGWINTKSGEATYVEEFKGRFVFSLDTSVSTAYLQISSLKAEDTAVY | |
| | | YCARWDFYDYVDEAMYWGQGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKRVEPKSCDK | |
| | VL-Ck | DIQMTQSPSSLSASVGDRVTITCKASQTVSANVAWYQQKPGKAPKLL | 117 |
| | | IYLASYRYRGVPSRFSGSGSGTDFTLTISSLQPEDFATYYCHQYYTY | |
| | | PLFTFGQGTKLEIKRTVAAPSVFIFPPSDEQLKSGTASVVCLLNNFY | |
| | | PREAKVQWKVDNALQSGNSQESVTEQDSKDSTYSLSSTLTLSKADYE | |
| | | KHKLYACEVTHQGLSSPVTKSENRGEC | |
| CTAT.07 | VH | NYGMN | 118 |
| (EpCAM) | CDR1 | | |
| | VH | WINTYTGESTYADSFKG | 119 |
| | CDR2 | | |
| | VH | FAIKGDY | 120 |
| | CDR3 | | |
| | VH | EVQLVQSGPGLVQPGGSVRISCAASGYTFTNYGMNWVKQAPGKGLEW | 121 |
| | | MGWINTYTGESTYADSFKGRFTFSLDTSASAAYLQINSLRAEDTAVY | |
| | | YCARFAIKGDYWGQGTLLTVSS | |
| | VL | RSTKSLLHSNGITYLY | 122 |
| | CDR1 | | |
| | VL | QMSNLAS | 123 |
| | CDR2 | | |
| | VL | AQNLEIPRT | 124 |
| | CDR3 | | |
| | VL | DIQMTQSPSSLSASVGDRVTITCRSTKSLLHSNGITYLYWYQQKPGK | 125 |
| | | APKLLIYQMSNLASGVPSRFSSSGSGTDFTLTISSLQPEDFATYYCA | |
| | | QNLEIPRTFGQGTKVELK | |
| | scFv | GGGSGGGEVQLVQSGPGLVQPGGSVRISCAASGYTFTNYGMNWVKQA | 126 |
| | | PGKGLEWMGWINTYTGESTYADSFKGRFTFSLDTSASAAYLQINSLR | 262 |
| | | AEDTAVYYCARFAIKGDYWGQGTLLTVSSGGGGSGGGGSGGGGSDIQ | (no N-terminus |
| | | MTQSPSSLSASVGDRVTITCRSTKSLLHSNGITYLYWYQQKPGKAPK | peptide |
| | | LLIYQMSNLASGVPSRFSSSGSGTDFTLTISSLQPEDFATYYCAQNL | linker) |
| | | EIPRTFGQGTKVELK | |
| | VH-CH1 | EVQLVQSGPGLVQPGGSVRISCAASGYTFTNYGMNWVKQAPGKGLEW | 127 |
| | | MGWINTYTGESTYADSFKGRFTFSLDTSASAAYLQINSLRAEDTAVY | |
| | | YCARFAIKGDYWGQGTLLTVSSASTKGPSVFPLAPSSKSTSGGTAAL | |
| | | GCLVKDYFPEPVTVSWNSGALTSGVHTFPAVLQSSGLYSLSSVVTVP | |
| | | SSSLGTQTYICNVNHKPSNTKVDKRVEPKSCDK | |
| | VL-Ck | DIQMTQSPSSLSASVGDRVTITCRSTKSLLHSNGITYLYWYQQKPGK | 128 |
| | | APKLLIYQMSNLASGVPSRFSSSGSGTDFTLTISSLQPEDFATYYCA | |
| | | QNLEIPRTFGQGTKVELKRTVAAPSVFIFPPSDEQLKSGTASVVCLL | |
| | | NNFYPREAKVQWKVDNALQSGNSQESVTEQDSKDSTYSLSSTLTLSK | |
| | | ADYEKHKLYACEVTHQGLSSPVTKSENRGEC | |
| CTAT.08 | VH | EYTIH | 129 |
| (FAP) | CDR1 | | |
| | VH | GINPNNGIPNYNQKFKG | 130 |
| | CDR2 | | |
| | VH | RRIAYGYDEGHAMDY | 131 |
| | CDR3 | | |
| | VH | QVQLVQSGAEVKKPGASVKVSCKTSRYTFTEYTIHWVRQAPGQRLEW | 132 |
| | | IGGINPNNGIPNYNQKFKGRVTITVDTSASTAYMELSSLRSEDTAVY | |
| | | YCARRRIAYGYDEGHAMDYWGQGTLVTVSS | |
| | VL | KSSQSLLYSRNQKNYLA | 133 |
| | CDR1 | | |
| | VL | WASTRES | 134 |
| | CDR2 | | |
| | VL | QQYFSYPLT | 135 |
| | CDR3 | | |
| | VL | DIVMTQSPDSLAVSLGERATINCKSSQSLLYSRNQKNYLAWYQQKPG | 136 |
| | | QPPKLLIFWASTRESGVPDRFSGSGFGTDFTLTISSLQAEDVAVYYC | |
| | | QQYFSYPLTFGQGTKVEIK | |
| | scFv | GGGSGGGQVQLVQSGAEVKKPGASVKVSCKTSRYTFTEYTIHWVRQA | 137 |
| | | PGQRLEWIGGINPNNGIPNYNQKFKGRVTITVDTSASTAYMELSSLR | 263 |
| | | SEDTAVYYCARRRIAYGYDEGHAMDYWGQGTLVTVSSGGGGSGGGGS | (no N-terminus |
| | | GGGGSDIVMTQSPDSLAVSLGERATINCKSSQSLLYSRNQKNYLAWY | peptide |
| | | QQKPGQPPKLLIFWASTRESGVPDRFSGSGFGTDFTLTISSLQAEDV | linker) |
| | | AVYYCQQYFSYPLTFGQGTKVEIK | |
| | VH-CH1 | QVQLVQSGAEVKKPGASVKVSCKTSRYTFTEYTIHWVRQAPGQRLEW | 138 |
| | | IGGINPNNGIPNYNQKFKGRVTITVDTSASTAYMELSSLRSEDTAVY | |
| | | YCARRRIAYGYDEGHAMDYWGQGTLVTVSSASTKGPSVFPLAPSSKS | |
| | | TSGGTAALGCLVKDYFPEPVTVSWNSGALTSGVHTFPAVLQSSGLYS | |
| | | LSSVVTVPSSSLGTQTYICNVNHKPSNTKVDKRVEPKSCDK | |
| | VL-Ck | DIVMTQSPDSLAVSLGERATINCKSSQSLLYSRNQKNYLAWYQQKPG | 139 |
| | | QPPKLLIFWASTRESGVPDRFSGSGFGTDFTLTISSLQAEDVAVYYC | |
| | | QQYFSYPLTFGQGTKVEIKRTVAAPSVFIFPPSDEQLKSGTASVVCL | |
| | | LNNFYPREAKVQWKVDNALQSGNSQESVTEQDSKDSTYSLSSTLTLS | |
| | | KADYEKHKLYACEVTHQGLSSPVTKSENRGEC | |
| CTAT.09 | VH | DSWIH | 140 |
| (PDL1) | CDR1 | | |
| | VH | WISPYGGSTYYADSVKG | 141 |
| | CDR2 | | |
| | VH | RHWPGGFDY | 142 |
| | CDR3 | | |
| | VH | EVQLVESGGGLVQPGGSLRLSCAASGFTFSDSWIHWVRQAPGKGLEW | 143 |
| | | VAWISPYGGSTYYADSVKGRFTISADTSKNTAYLQMNSLRAEDTAVY | |
| | | YCARRHWPGGFDYWGQGTLVTVSS | |
| | VL | RASQDVSTAVA | 144 |
| | CDR1 | | |
| | VL | SASFLYS | 145 |
| | CDR2 | | |
| | VL | QQYLYHPAT | 146 |
| | CDR3 | | |
| | VL | DIQMTQSPSSLSASVGDRVTITCRASQDVSTAVAWYQQKPGKAPKLL | 147 |
| | | IYSASFLYSGVPSRFSGSGSGTDFTLTISSLQPEDFATYYCQQYLYH | |
| | | PATFGQGTKVEIK | |
| | scFv | GGGSGGGEVQLVESGGGLVQPGGSLRLSCAASGFTFSDSWIHWVRQA | 148 |
| | | PGKGLEWVAWISPYGGSTYYADSVKGRFTISADTSKNTAYLQMNSLR | 264 |
| | | AEDTAVYYCARRHWPGGFDYWGQGTLVTVSSGGGGSGGGGSGGGGSD | (no N-terminus |
| | | IQMTQSPSSLSASVGDRVTITCRASQDVSTAVAWYQQKPGKAPKLLI | peptide |
| | | YSASFLYSGVPSRFSGSGSGTDFTLTISSLQPEDFATYYCQQYLYHP | linker) |
| | | ATFGQGTKVEIK | |
| | VH-CH1 | EVQLVESGGGLVQPGGSLRLSCAASGFTFSDSWIHWVRQAPGKGLEW | 149 |
| | | VAWISPYGGSTYYADSVKGRFTISADTSKNTAYLQMNSLRAEDTAVY | |
| | | YCARRHWPGGFDYWGQGTLVTVSSASTKGPSVFPLAPSSKSTSGGTA | |
| | | ALGCLVKDYFPEPVTVSWNSGALTSGVHTFPAVLQSSGLYSLSSVVT | |
| | | VPSSSLGTQTYICNVNHKPSNTKVDKKVEPKSCDK | |
| | VL-Ck | DIQMTQSPSSLSASVGDRVTITCRASQDVSTAVAWYQQKPGKAPKLL | 150 |
| | | IYSASFLYSGVPSRFSGSGSGTDFTLTISSLQPEDFATYYCQQYLYH | |
| | | PATFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| CTAT.10 | VH | SFAMS | 151 |
| (CD38) | CDR1 | | |
| | VH | AISGSGGGTYYADSVKG | 152 |
| | CDR2 | | |
| | VH | DKILWFGEPVFDY | 153 |
| | CDR3 | | |
| | VH | EVQLLESGGGLVQPGGSLRLSCAVSGFTFNSFAMSWVRQAPGKGLEW | 154 |
| | | VSAISGSGGGTYYADSVKGRFTISRDNSKNTLYLQMNSLRAEDTAVY | |
| | | FCAKDKILWFGEPVFDYWGQGTLVTVSS | |
| | VL | RASQSVSSYLA | 155 |
| | CDR1 | | |
| | VL | DASNRAT | 156 |
| | CDR2 | | |
| | VL | QQRSNWPPT | 157 |
| | CDR3 | | |
| | VL | EIVLTQSPATLSLSPGERATLSCRASQSVSSYLAWYQQKPGQAPRLL | 158 |
| | | IYDASNRATGIPARFSGSGSGTDFTLTISSLEPEDFAVYYCQQRSNW | |
| | | PPTFGQGTKVEIK | |
| | scFv | GGGSGGGEVQLLESGGGLVQPGGSLRLSCAVSGFTFNSFAMSWVRQA | 159 |
| | | PGKGLEWVSAISGSGGGTYYADSVKGRFTISRDNSKNTLYLQMNSLR | 265 |
| | | AEDTAVYFCAKDKILWFGEPVFDYWGQGTLVTVSSGGGGSGGGGSGG | (no N-terminus |
| | | GGSEIVLTQSPATLSLSPGERATLSCRASQSVSSYLAWYQQKPGQAP | peptide |
| | | RLLIYDASNRATGIPARFSGSGSGTDFTLTISSLEPEDFAVYYCQQR | linker) |
| | | SNWPPTFGQGTKVEIK | |
| | VH-CH1 | EVQLLESGGGLVQPGGSLRLSCAVSGFTFNSFAMSWVRQAPGKGLEW | 160 |
| | | VSAISGSGGGTYYADSVKGRFTISRDNSKNTLYLQMNSLRAEDTAVY | |
| | | FCAKDKILWFGEPVFDYWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDK | |
| | VL-Ck | EIVLTQSPATLSLSPGERATLSCRASQSVSSYLAWYQQKPGQAPRLL | 161 |
| | | IYDASNRATGIPARFSGSGSGTDFTLTISSLEPEDFAVYYCQQRSNW | |
| | | PPTFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| CTAT.11 | VH | DSNIH | 162 |
| (CD33) | CDR1 | | |
| | VH | YIYPYNGGTDYNQKFKN | 163 |
| | CDR2 | | |
| | VH | GNPWLAY | 164 |
| | CDR3 | | |
| | VH | EVQLVQSGAEVKKPGSSVKVSCKASGYTITDSNIHWVRQAPGQSLEW | 165 |
| | | IGYIYPYNGGTDYNQKFKNRATLTVDNPTNTAYMELSSLRSEDTAFY | |
| | | YCVNGNPWLAYWGQGTLVTVSS | |
| | VL | RASESLDNYGIRFLT | 166 |
| | CDR1 | | |
| | VL | AASNQGS | 167 |
| | CDR2 | | |
| | VL | QQTKEVPWS | 168 |
| | CDR3 | | |
| | VL | DIQLTQSPSTLSASVGDRVTITCRASESLDNYGIRFLTWFQQKPGKA | 169 |
| | | PKLLMYAASNQGSGVPSRFSGSGSGTEFTLTISSLQPDDFATYYCQQ | |
| | | TKEVPWSFGQGTKVEVK | |
| | scFv | GGGSGGGEVQLVQSGAEVKKPGSSVKVSCKASGYTITDSNIHWVRQA | 170 |
| | | PGQSLEWIGYIYPYNGGTDYNQKFKNRATLTVDNPTNTAYMELSSLR | 266 |
| | | SEDTAFYYCVNGNPWLAYWGQGTLVTVSSGGGGSGGGGSGGGGSDIQ | (no N-terminus |
| | | LTQSPSTLSASVGDRVTITCRASESLDNYGIRFLTWFQQKPGKAPKL | peptide |
| | | LMYAASNQGSGVPSRFSGSGSGTEFTLTISSLQPDDFATYYCQQTKE | linker) |
| | | VPWSFGQGTKVEVK | |
| | VH-CH1 | EVQLVQSGAEVKKPGSSVKVSCKASGYTITDSNIHWVRQAPGQSLEW | 171 |
| | | IGYIYPYNGGTDYNQKFKNRATLTVDNPTNTAYMELSSLRSEDTAFY | |
| | | YCVNGNPWLAYWGQGTLVTVSSASTKGPSVFPLAPCSRSTSESTAAL | |
| | | GCLVKDYFPEPVTVSWNSGALTSGVHTFPAVLQSSGLYSLSSVVTVP | |
| | | SSSLGTKTYTCNVDHKPSNTKVDKRV | |
| | VL-Ck | DIQLTQSPSTLSASVGDRVTITCRASESLDNYGIRFLTWFQQKPGKA | 172 |
| | | PKLLMYAASNQGSGVPSRFSGSGSGTEFTLTISSLQPDDFATYYCQQ | |
| | | TKEVPWSFGQGTKVEVKRTVAAPSVFIFPPSDEQLKSGTASVVCLLN | |
| | | NFYPREAKVQWKVDNALQSGNSQESVTEQDSKDSTYSLSSTLTLSKA | |
| | | DYEKHKVYACEVTHQGLSSPVTKSENRGEC | |
| CTAT.12 | VH | SYWLH | 173 |
| (HGFR (cMET)) | CDR1 | | |
| | VH | MIDPSNSDTRFNPNFKD | 174 |
| | CDR2 | | |
| | VH | YRSYVTPLDY | 175 |
| | CDR3 | | |
| | VH | EVQLVESGGGLVQPGGSLRLSCAASGYTFTSYWLHWVRQAPGKGLEW | 176 |
| | | VGMIDPSNSDTRFNPNFKDRFTISADTSKNTAYLQMNSLRAEDTAVY | |
| | | YCATYRSYVTPLDYWGQGTLVTVSS | |
| | VL | KSSQSLLYTSSQKNYLA | 177 |
| | CDR1 | | |
| | VL | WASTRES | 178 |
| | CDR2 | | |
| | VL | QQYYAYPWT | 179 |
| | CDR3 | | |
| | VL | DIQMTQSPSSLSASVGDRVTITCKSSQSLLYTSSQKNYLAWYQQKPG | 180 |
| | | KAPKLLIYWASTRESGVPSRFSGSGSGTDFTLTISSLQPEDFATYYC | |
| | | QQYYAYPWTFGQGTKVEIK | |
| | scFv | GGGSGGGEVQLVESGGGLVQPGGSLRLSCAASGYTFTSYWLHWVRQA | 181 |
| | | PGKGLEWVGMIDPSNSDTRENPNFKDRFTISADTSKNTAYLQMNSLR | 267 |
| | | AEDTAVYYCATYRSYVTPLDYWGQGTLVTVSSGGGGSGGGGSGGGGS | (no N-terminus |
| | | DIQMTQSPSSLSASVGDRVTITCKSSQSLLYTSSQKNYLAWYQQKPG | peptide |
| | | KAPKLLIYWASTRESGVPSRFSGSGSGTDFTLTISSLQPEDFATYYC | linker) |
| | | QQYYAYPWTFGQGTKVEIK | |
| | VH-CH1 | EVQLVESGGGLVQPGGSLRLSCAASGYTFTSYWLHWVRQAPGKGLEW | 182 |
| | | VGMIDPSNSDTRFNPNFKDRFTISADTSKNTAYLQMNSLRAEDTAVY | |
| | | YCATYRSYVTPLDYWGQGTLVTVSSASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVDKKVEPKSCDK | |
| | VL-Ck | DIQMTQSPSSLSASVGDRVTITCKSSQSLLYTSSQKNYLAWYQQKPG | 183 |
| | | KAPKLLIYWASTRESGVPSRFSGSGSGTDFTLTISSLQPEDFATYYC | |
| | | QQYYAYPWTFGQGTKVEIKRTVAAPSVFIFPPSDEQLKSGTASVVCL | |
| | | LNNFYPREAKVQWKVDNALQSGNSQESVTEQDSKDSTYSLSSTLTLS | |
| | | KADYEKHKVYACEVTHQGLSSPVTKSENRGEC | |
| CTAT.13 | VH | NYNMH | 184 |
| (CD47) | CDR1 | | |
| | VH | TIYPGNDDTSYNQKFKD | 185 |
| | CDR2 | | |
| | VH | GGYRAMDY | 186 |
| | CDR3 | | |
| | VH | QVQLVQSGAEVKKPGASVKVSCKASGYTFTNYNMHWVRQAPGQRLEW | 187 |
| | | MGTIYPGNDDTSYNQKFKDRVTITADTSASTAYMELSSLRSEDTAVY | |
| | | YCARGGYRAMDYWGQGTLVTVSS | |
| | VL | RSSQSIVYSNGNTYLG | 188 |
| | CDR1 | | |
| | VL | KVSNRFS | 189 |
| | CDR2 | | |
| | VL | FQGSHVPYT | 190 |
| | CDR3 | | |
| | VL | DIVMTQSPLSLPVTPGEPASISCRSSQSIVYSNGNTYLGWYLQKPGQ | 191 |
| | | SPQLLIYKVSNRFSGVPDRFSGSGSGTDFTLKISRVEAEDVGVYYCF | |
| | | QGSHVPYTFGQGTKLEIK | |
| | scFv | GGGSGGGQVQLVQSGAEVKKPGASVKVSCKASGYTFTNYNMHWVRQA | 192 |
| | | PGQRLEWMGTIYPGNDDTSYNQKFKDRVTITADTSASTAYMELSSLR | 268 |
| | | SEDTAVYYCARGGYRAMDYWGQGTLVTVSSGGGGSGGGGSGGGGSDI | (no N-terminus |
| | | VMTQSPLSLPVTPGEPASISCRSSQSIVYSNGNTYLGWYLQKPGQSP | peptide |
| | | QLLIYKVSNRFSGVPDRFSGSGSGTDFTLKISRVEAEDVGVYYCFQG | linker) |
| | | SHVPYTFGQGTKLEIK | |
| | VH-CH1 | QVQLVQSGAEVKKPGASVKVSCKASGYTFTNYNMHWVRQAPGQRLEW | 193 |
| | | MGTIYPGNDDTSYNQKFKDRVTITADTSASTAYMELSSLRSEDTAVY | |
| | | YCARGGYRAMDYWGQGTLVTVSSASTKGPSVFPLAPSSKSTSGGTAA | |
| | | LGCLVKDYFPEPVTVSWNSGALTSGVHTFPAVLQSSGLYSLSSVVTV | |
| | | PSSSLGTQTYICNVNHKPSNTKVDKRVEPKSCDK | |
| | VL-Ck | DIVMTQSPLSLPVTPGEPASISCRSSQSIVYSNGNTYLGWYLQKPGQ | 194 |
| | | SPQLLIYKVSNRFSGVPDRFSGSGSGTDFTLKISRVEAEDVGVYYCF | |
| | | QGSHVPYTFGQGTKLEIKRTVAAPSVFIFPPSDEQLKSGTASVVCLL | |
| | | NNFYPREAKVQWKVDNALQSGNSQESVTEQDSKDSTYSLSSTLTLSK | |
| | | ADYEKHKVYACEVTHQGLSSPVTKSENRGEC | |
| CTAT.14 | VH | SGDYFWS | 195 |
| (TRAIL-R2) | CDR1 | | |
| | VH | HIHNSGTTYYNPSLKS | 196 |
| | CDR2 | | |
| | VH | DRGGDYYYGMDV | 197 |
| | CDR3 | | |
| | VH | QVQLQESGPGLVKPSQTLSLTCTVSGGSISSGDYFWSWIRQLPGKGL | 198 |
| | | EWIGHIHNSGTTYYNPSLKSRVTISVDTSKKQFSLRLSSVTAADTAV | |
| | | YYCARDRGGDYYYGMDVWGQGTTVTVSS | |
| | VL | RASQGISRSYLA | 199 |
| | CDR1 | | |
| | VL | GASSRAT | 200 |
| | CDR2 | | |
| | VL | QQFGSSPWI | 201 |
| | CDR3 | | |
| | VL | EIVLTQSPGTLSLSPGERATLSCRASQGISRSYLAWYQQKPGQAPSL | 202 |
| | | LIYGASSRATGIPDRFSGSGSGTDFTLTISRLEPEDFAVYYCQQFGS | |
| | | SPWTFGQGTKVEIK | |
| | scFv | GGGSGGGQVQLQESGPGLVKPSQTLSLTCTVSGGSISSGDYFWSWIR | 203 |
| | | QLPGKGLEWIGHIHNSGTTYYNPSLKSRVTISVDTSKKQFSLRLSSV | 269 |
| | | TAADTAVYYCARDRGGDYYYGMDVWGQGTTVTVSSGGGGSGGGGSGG | (no N-terminus |
| | | GGSEIVLTQSPGTLSLSPGERATLSCRASQGISRSYLAWYQQKPGQA | peptide |
| | | PSLLIYGASSRATGIPDRFSGSGSGTDFTLTISRLEPEDFAVYYCQQ | linker) |
| | | FGSSPWTFGQGTKVEIK | |
| | VH-CH1 | QVQLQESGPGLVKPSQTLSLTCTVSGGSISSGDYFWSWIRQLPGKGL | 204 |
| | | EWIGHIHNSGTTYYNPSLKSRVTISVDTSKKQFSLRLSSVTAADTAV | |
| | | YYCARDRGGDYYYGMDVWGQGTTVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDK | |
| | VL-Ck | EIVLTQSPGTLSLSPGERATLSCRASQGISRSYLAWYQQKPGQAPSL | 205 |
| | | LIYGASSRATGIPDRESGSGSGTDFTLTISRLEPEDFAVYYCQQFGS | |
| | | SPWTFGQGTKVEIKRTVAAPSVFIFPPSDEQLKSGTASVVCLLNNFY | |
| | | PREAKVQWKVDNALQSGNSQESVTEQDSKDSTYSLSSTLTLSKADYE | |
| | | KHKVYACEVTHQGLSSPVTKSENRGEC | |
| CTAT.15 | VH | SGSYYWS | 206 |
| (mesothelin) | CDR1 | | |
| | VH | YIYYSGSTNYNPSLKS | 207 |
| | CDR2 | | |
| | VH | EGKNGAFDI | 208 |
| | CDR3 | | |
| | VH | QVQLQESGPGLVKPSETLSLTCTVSGGSVSSGSYYWSWIRQPPGKGL | 209 |
| | | EWIGYIYYSGSTNYNPSLKSRVTISVDTSKNQFSLKLSSVTAADTAV | |
| | | YYCAREGKNGAFDIWGQGTMVTVSS | |
| | VL | RASQSISSYLN | 210 |
| | CDR1 | | |
| | VL | AASSLQS | 211 |
| | CDR2 | | |
| | VL | QQSYSTPLT | 212 |
| | CDR3 | | |
| | VL | DIQMTQSPSSLSASVGDRVTITCRASQSISSYLNWYQQKPGKAPKLL | 213 |
| | | IYAASSLQSGVPSGFSGSGSGTDFTLTISSLQPEDFATYYCQQSYST | |
| | | PLTFGGGTKVEIK | |
| | scFv | GGGSGGGQVQLQESGPGLVKPSETLSLTCTVSGGSVSSGSYYWSWIR | 214 |
| | | QPPGKGLEWIGYIYYSGSTNYNPSLKSRVTISVDTSKNQFSLKLSSV | 270 |
| | | TAADTAVYYCAREGKNGAFDIWGQGTMVTVSSGGGGSGGGGSGGGGS | (no N-terminus |
| | | DIQMTQSPSSLSASVGDRVTITCRASQSISSYLNWYQQKPGKAPKLL | peptide |
| | | IYAASSLQSGVPSGFSGSGSGTDFTLTISSLQPEDFATYYCQQSYST | linker) |
| | | PLTFGGGTKVEIK | |
| | VH-CH1 | QVQLQESGPGLVKPSETLSLTCTVSGGSVSSGSYYWSWIRQPPGKGL | 215 |
| | | EWIGYIYYSGSTNYNPSLKSRVTISVDTSKNQFSLKLSSVTAADTAV | |
| | | YYCAREGKNGAFDIWGQGTMVTVSSASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVDKKVEPKSCDK | |
| | VL-Ck | DIQMTQSPSSLSASVGDRVTITCRASQSISSYLNWYQQKPGKAPKLL | 216 |
| | | IYAASSLQSGVPSGFSGSGSGTDFTLTISSLQPEDFATYYCQQSYST | |
| | | PLTFGGGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSENRGEC | |
| CTAT.16 | VH | GYNMN | 217 |
| (GD2) | CDR1 | | |
| | VH | AIDPYYGGTSYNQKFKG | 218 |
| | CDR2 | | |
| | VH | GMEY | 219 |
| | CDR3 | | |
| | VH | EVQLLQSGPELEKPGASVMISCKASGSSFTGYNMNWVRQNIGKSLEW | 220 |
| | | IGAIDPYYGGTSYNQKFKGRATLTVDKSSSTAYMHLKSLTSEDSAVY | |
| | | YCVSGMEYWGQGTSVTVSS | |
| | VL | RSSQSLVHRNGNTYLH | 221 |
| | CDR1 | | |
| | VL | KVSNRFS | 222 |
| | CDR2 | | |
| | VL | SQSTHVPPLT | 223 |
| | CDR3 | | |
| | VL | EIVMTQSPATLSVSPGERATLSCRSSQSLVHRNGNTYLHWYLQKPGQ | 224 |
| | | SPKLLIHKVSNRFSGVPDRFSGSGSGTDFTLKISRVEAEDLGVYFCS | |
| | | QSTHVPPLTFGAGTKLELK | |
| | scFv | GGGSGGGEVQLLQSGPELEKPGASVMISCKASGSSFTGYNMNWVRQN | 225 |
| | | IGKSLEWIGAIDPYYGGTSYNQKFKGRATLTVDKSSSTAYMHLKSLT | |
| | | SEDSAVYYCVSGMEYWGQGTSVTVSSGGGGSGGGGSGGGGSEIVMTQ | 271 |
| | | SPATLSVSPGERATLSCRSSQSLVHRNGNTYLHWYLQKPGQSPKLLI | (no N-terminus |
| | | HKVSNRFSGVPDRFSGSGSGTDFTLKISRVEAEDLGVYFCSQSTHVP | peptide |
| | | PLTFGAGTKLELK | linker) |
| | VH-CH1 | EVQLLQSGPELEKPGASVMISCKASGSSFTGYNMNWVRQNIGKSLEW | 226 |
| | | IGAIDPYYGGTSYNQKFKGRATLTVDKSSSTAYMHLKSLTSEDSAVY | |
| | | YCVSGMEYWGQGTSVTVSSASTKGPSVFPLAPSSKSTSGGTAALGCL | |
| | | VKDYFPEPVTVSWNSGALTSGVHTFPAVLQSSGLYSLSSVVTVPSSS | |
| | | LGTQTYICNVNHKPSNTKVDKRVEPKSCDK | |
| | VL-Ck | EIVMTQSPATLSVSPGERATLSCRSSQSLVHRNGNTYLHWYLQKPGQ | 227 |
| | | SPKLLIHKVSNRFSGVPDRFSGSGSGTDFTLKISRVEAEDLGVYFCS | |
| | | QSTHVPPLTFGAGTKLELKRTVAAPSVFIFPPSDEQLKSGTASVVCL | |
| | | LNNFYPREAKVQWKVDNALQSGNSQESVTEQDSKDSTYSLSSTLTLS | |
| | | KADYEKHKVYACEVTHQGLSSPVTKSENRGEC | |

In some examples, the anti-TAA binding moiety may comprise the same heavy chain CDRs as those in antibody CTAT.01, which are provided in Table 2 above. Alternatively or in addition, the anti-TAA binding moiety may have the same light chain CDRs as those in antibody CTAT.01, which are also provided in Table 2 above. Such an anti-TAA binding moiety may comprise the same V_{H} and/or V_{L} chains as CTAT.01. Alternatively, the anti-TAA binding moiety may comprise amino acid variations in one or more of the framework regions relative to the corresponding framework regions in CTAT.01. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.01.

In some embodiments, the anti-TAA moiety may comprise a certain level of variations in one or more of the CDRs relative to those of CTAT.01. For example, the anti-TAA moiety may comprise heavy chain CDRs that are at least 80% (e.g., 85%, 90%, 95%, or 98%) sequence identity, individually or collectively, as compared with the V_{H} CDRs of CTAT.01. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.01.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.01. In some instances, the anti-TAA moiety may comprise the same heavy chain CDR3 as the heavy chain CDR3 of CTAT.01 and comprise one or more amino acid variations in one or more of the other heavy chain and light chain CDRs.

In some examples, the anti-TAA binding moiety may comprise the same heavy chain CDRs as those in antibody CTAT.02, which are provided in Table 2 above. Alternatively or in addition, the anti-TAA binding moiety may have the same light chain CDRs as those in antibody CTAT.02, which are also provided in Table 2 above. Such an anti-TAA binding moiety may comprise the same V_{H} and/or V_{L} chains as CTAT.02. Alternatively, the anti-TAA binding moiety may comprise amino acid variations in one or more of the framework regions relative to the corresponding framework regions in CTAT.02. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.02.

In one specific example, the anti-TAA moiety disclosed herein comprises a mutation at position G42 of the V_{L} chain relative to CTAT.02, for example, an amino acid residue substitution (e.g., G42A). See, e.g., CTAT.02 VL-01 in Table 2 above. In another specific example, the anti-TAA moiety disclosed herein comprises a mutation at position D41 of the V_{L} chain relative to CTAT.02, for example, an amino acid residue substitution (e.g., D41E). See, e.g., CTAT.02 VL-02 in Table 2 above.

In some embodiments, the anti-TAA moiety may comprise a certain level of variations in one or more of the CDRs relative to those of CTAT.02. For example, the anti-TAA moiety may comprise heavy chain CDRs that are at least 80% (e.g., 85%, 90%, 95%, or 98%) sequence identity, individually or collectively, as compared with the V_{H} CDRs of CTAT.02. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.02.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.02. In some instances, the anti-TAA moiety may comprise the same heavy chain CDR3 as the heavy chain CDR3 of CTAT.02 and comprise one or more amino acid variations in one or more of the other heavy chain and light chain CDRs.

In some examples, the anti-TAA binding moiety may comprise the same heavy chain CDRs as those in antibody CTAT.03, which are provided in Table 2 above. Alternatively or in addition, the anti-TAA binding moiety may have the same light chain CDRs as those in antibody CTAT.03, which are also provided in Table 2 above. Such an anti-TAA binding moiety may comprise the same V_{H} and/or V_{L} chains as CTAT.03. Alternatively, the anti-TAA binding moiety may comprise amino acid variations in one or more of the framework regions relative to the corresponding framework regions in CTAT.03. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.03.

In some embodiments, the anti-TAA moiety may comprise a certain level of variations in one or more of the CDRs relative to those of CTAT.03. For example, the anti-TAA moiety may comprise heavy chain CDRs that are at least 80% (e.g., 85%, 90%, 95%, or 98%) sequence identity, individually or collectively, as compared with the V_{H} CDRs of CTAT.03. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.03.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.03. In some instances, the anti-TAA moiety may comprise the same heavy chain CDR3 as the heavy chain CDR3 of CTAT.03 and comprise one or more amino acid variations in one or more of the other heavy chain and light chain CDRs.

In some examples, the anti-TAA binding moiety may comprise the same heavy chain CDRs as those in antibody CTAT.04, which are provided in Table 2 above. Alternatively or in addition, the anti-TAA binding moiety may have the same light chain CDRs as those in antibody CTAT.04, which are also provided in Table 2 above. Such an anti-TAA binding moiety may comprise the same V_{H} and/or V_{L} chains as CTAT.04. Alternatively, the anti-TAA binding moiety may comprise amino acid variations in one or more of the framework regions relative to the corresponding framework regions in CTAT.04. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.04.

In some embodiments, the anti-TAA moiety may comprise a certain level of variations in one or more of the CDRs relative to those of CTAT.04. For example, the anti-TAA moiety may comprise heavy chain CDRs that are at least 80% (e.g., 85%, 90%, 95%, or 98%) sequence identity, individually or collectively, as compared with the V_{H} CDRs of CTAT.04. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.04.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.04. In some instances, the anti-TAA moiety may comprise the same heavy chain CDR3 as the heavy chain CDR3 of CTAT.04 and comprise one or more amino acid variations in one or more of the other heavy chain and light chain CDRs.

In some examples, the anti-TAA binding moiety may comprise the same heavy chain CDRs as those in antibody CTAT.05, which are provided in Table 2 above. Alternatively or in addition, the anti-TAA binding moiety may have the same light chain CDRs as those in antibody CTAT.05, which are also provided in Table 2 above. Such an anti-TAA binding moiety may comprise the same V_{H} and/or V_{L} chains as CTAT.05. Alternatively, the anti-TAA binding moiety may comprise amino acid variations in one or more of the framework regions relative to the corresponding framework regions in CTAT.05. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.05.

In some embodiments, the anti-TAA moiety may comprise a certain level of variations in one or more of the CDRs relative to those of CTAT.05. For example, the anti-TAA moiety may comprise heavy chain CDRs that are at least 80% (e.g., 85%, 90%, 95%, or 98%) sequence identity, individually or collectively, as compared with the V_{H} CDRs of CTAT.05. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.05.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.05. In some instances, the anti-TAA moiety may comprise the same heavy chain CDR3 as the heavy chain CDR3 of CTAT.05 and comprise one or more amino acid variations in one or more of the other heavy chain and light chain CDRs.

In some examples, the anti-TAA binding moiety may comprise the same heavy chain CDRs as those in antibody CTAT.06, which are provided in Table 2 above. Alternatively or in addition, the anti-TAA binding moiety may have the same light chain CDRs as those in antibody CTAT.06, which are also provided in Table 2 above. Such an anti-TAA binding moiety may comprise the same V_{H} and/or V_{L} chains as CTAT.06. Alternatively, the anti-TAA binding moiety may comprise amino acid variations in one or more of the framework regions relative to the corresponding framework regions in CTAT.06. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.06.

In some embodiments, the anti-TAA moiety may comprise a certain level of variations in one or more of the CDRs relative to those of CTAT.06. For example, the anti-TAA moiety may comprise heavy chain CDRs that are at least 80% (e.g., 85%, 90%, 95%, or 98%) sequence identity, individually or collectively, as compared with the V_{H} CDRs of CTAT.06. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.06.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.06. In some instances, the anti-TAA moiety may comprise the same heavy chain CDR3 as the heavy chain CDR3 of CTAT.06 and comprise one or more amino acid variations in one or more of the other heavy chain and light chain CDRs.

In some examples, the anti-TAA binding moiety may comprise the same heavy chain CDRs as those in antibody CTAT.07, which are provided in Table 2 above. Alternatively or in addition, the anti-TAA binding moiety may have the same light chain CDRs as those in antibody CTAT.07, which are also provided in Table 2 above. Such an anti-TAA binding moiety may comprise the same V_{H} and/or V_{L} chains as CTAT.07. Alternatively, the anti-TAA binding moiety may comprise amino acid variations in one or more of the framework regions relative to the corresponding framework regions in CTAT.07. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.07.

In some embodiments, the anti-TAA moiety may comprise a certain level of variations in one or more of the CDRs relative to those of CTAT.07. For example, the anti-TAA moiety may comprise heavy chain CDRs that are at least 80% (e.g., 85%, 90%, 95%, or 98%) sequence identity, individually or collectively, as compared with the V_{H} CDRs of CTAT.07. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.07.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.07. In some instances, the anti-TAA moiety may comprise the same heavy chain CDR3 as the heavy chain CDR3 of CTAT.07 and comprise one or more amino acid variations in one or more of the other heavy chain and light chain CDRs.

In some examples, the anti-TAA binding moiety may comprise the same heavy chain CDRs as those in antibody CTAT.08, which are provided in Table 2 above. Alternatively or in addition, the anti-TAA binding moiety may have the same light chain CDRs as those in antibody CTAT.08, which are also provided in Table 2 above. Such an anti-TAA binding moiety may comprise the same V_{H} and/or V_{L} chains as CTAT.08. Alternatively, the anti-TAA binding moiety may comprise amino acid variations in one or more of the framework regions relative to the corresponding framework regions in CTAT.08. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.08.

In some embodiments, the anti-TAA moiety may comprise a certain level of variations in one or more of the CDRs relative to those of CTAT.08. For example, the anti-TAA moiety may comprise heavy chain CDRs that are at least 80% (e.g., 85%, 90%, 95%, or 98%) sequence identity, individually or collectively, as compared with the V_{H} CDRs of CTAT.08. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.08.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.08. In some instances, the anti-TAA moiety may comprise the same heavy chain CDR3 as the heavy chain CDR3 of CTAT.08 and comprise one or more amino acid variations in one or more of the other heavy chain and light chain CDRs.

In some examples, the anti-TAA binding moiety may comprise the same heavy chain CDRs as those in antibody CTAT.09, which are provided in Table 2 above. Alternatively or in addition, the anti-TAA binding moiety may have the same light chain CDRs as those in antibody CTAT.09, which are also provided in Table 2 above. Such an anti-TAA binding moiety may comprise the same V_{H} and/or V_{L} chains as CTAT.09. Alternatively, the anti-TAA binding moiety may comprise amino acid variations in one or more of the framework regions relative to the corresponding framework regions in CTAT.09. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.09.

In some embodiments, the anti-TAA moiety may comprise a certain level of variations in one or more of the CDRs relative to those of CTAT.09. For example, the anti-TAA moiety may comprise heavy chain CDRs that are at least 80% (e.g., 85%, 90%, 95%, or 98%) sequence identity, individually or collectively, as compared with the V_{H} CDRs of CTAT.09. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.09.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.09. In some instances, the anti-TAA moiety may comprise the same heavy chain CDR3 as the heavy chain CDR3 of CTAT.09 and comprise one or more amino acid variations in one or more of the other heavy chain and light chain CDRs.

In some examples, the anti-TAA binding moiety may comprise the same heavy chain CDRs as those in antibody CTAT.10, which are provided in Table 2 above. Alternatively or in addition, the anti-TAA binding moiety may have the same light chain CDRs as those in antibody CTAT.10, which are also provided in Table 2 above. Such an anti-TAA binding moiety may comprise the same V_{H} and/or V_{L} chains as CTAT.10. Alternatively, the anti-TAA binding moiety may comprise amino acid variations in one or more of the framework regions relative to the corresponding framework regions in CTAT.10. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.10.

In some embodiments, the anti-TAA moiety may comprise a certain level of variations in one or more of the CDRs relative to those of CTAT.10. For example, the anti-TAA moiety may comprise heavy chain CDRs that are at least 80% (e.g., 85%, 90%, 95%, or 98%) sequence identity, individually or collectively, as compared with the V_{H} CDRs of CTAT.10. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.10.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.10. In some instances, the anti-TAA moiety may comprise the same heavy chain CDR3 as the heavy chain CDR3 of CTAT.10 and comprise one or more amino acid variations in one or more of the other heavy chain and light chain CDRs.

In some examples, the anti-TAA binding moiety may comprise the same heavy chain CDRs as those in antibody CTAT.11, which are provided in Table 2 above. Alternatively or in addition, the anti-TAA binding moiety may have the same light chain CDRs as those in antibody CTAT.11, which are also provided in Table 2 above. Such an anti-TAA binding moiety may comprise the same V_{H} and/or V_{L} chains as CTAT.11. Alternatively, the anti-TAA binding moiety may comprise amino acid variations in one or more of the framework regions relative to the corresponding framework regions in CTAT.11. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.11.

In some embodiments, the anti-TAA moiety may comprise a certain level of variations in one or more of the CDRs relative to those of CTAT.11. For example, the anti-TAA moiety may comprise heavy chain CDRs that are at least 80% (e.g., 85%, 90%, 95%, or 98%) sequence identity, individually or collectively, as compared with the V_{H} CDRs of CTAT.11. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.10.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.11. In some instances, the anti-TAA moiety may comprise the same heavy chain CDR3 as the heavy chain CDR3 of CTAT.11 and comprise one or more amino acid variations in one or more of the other heavy chain and light chain CDRs.

In some examples, the anti-TAA binding moiety may comprise the same heavy chain CDRs as those in antibody CTAT.12, which are provided in Table 2 above. Alternatively or in addition, the anti-TAA binding moiety may have the same light chain CDRs as those in antibody CTAT.12, which are also provided in Table 2 above. Such an anti-TAA binding moiety may comprise the same V_{H} and/or V_{L} chains as CTAT.12. Alternatively, the anti-TAA binding moiety may comprise amino acid variations in one or more of the framework regions relative to the corresponding framework regions in CTAT.12. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.12.

In some embodiments, the anti-TAA moiety may comprise a certain level of variations in one or more of the CDRs relative to those of CTAT.12. For example, the anti-TAA moiety may comprise heavy chain CDRs that are at least 80% (e.g., 85%, 90%, 95%, or 98%) sequence identity, individually or collectively, as compared with the V_{H} CDRs of CTAT.12. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.12.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.12. In some instances, the anti-TAA moiety may comprise the same heavy chain CDR3 as the heavy chain CDR3 of CTAT.12 and comprise one or more amino acid variations in one or more of the other heavy chain and light chain CDRs.

In some examples, the anti-TAA binding moiety may comprise the same heavy chain CDRs as those in antibody CTAT.13, which are provided in Table 2 above. Alternatively or in addition, the anti-TAA binding moiety may have the same light chain CDRs as those in antibody CTAT.13, which are also provided in Table 2 above. Such an anti-TAA binding moiety may comprise the same V_{H} and/or V_{L} chains as CTAT.13. Alternatively, the anti-TAA binding moiety may comprise amino acid variations in one or more of the framework regions relative to the corresponding framework regions in CTAT.13. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.13.

In some embodiments, the anti-TAA moiety may comprise a certain level of variations in one or more of the CDRs relative to those of CTAT.13. For example, the anti-TAA moiety may comprise heavy chain CDRs that are at least 80% (e.g., 85%, 90%, 95%, or 98%) sequence identity, individually or collectively, as compared with the V_{H} CDRs of CTAT.13. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.13.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.13. In some instances, the anti-TAA moiety may comprise the same heavy chain CDR3 as the heavy chain CDR3 of CTAT.13 and comprise one or more amino acid variations in one or more of the other heavy chain and light chain CDRs.

In some examples, the anti-TAA binding moiety may comprise the same heavy chain CDRs as those in antibody CTAT.14, which are provided in Table 2 above. Alternatively or in addition, the anti-TAA binding moiety may have the same light chain CDRs as those in antibody CTAT.14, which are also provided in Table 2 above. Such an anti-TAA binding moiety may comprise the same V_{H} and/or V_{L} chains as CTAT.14. Alternatively, the anti-TAA binding moiety may comprise amino acid variations in one or more of the framework regions relative to the corresponding framework regions in CTAT.14. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.14.

In some embodiments, the anti-TAA moiety may comprise a certain level of variations in one or more of the CDRs relative to those of CTAT.14. For example, the anti-TAA moiety may comprise heavy chain CDRs that are at least 80% (e.g., 85%, 90%, 95%, or 98%) sequence identity, individually or collectively, as compared with the V_{H} CDRs of CTAT.14. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.14.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.14. In some instances, the anti-TAA moiety may comprise the same heavy chain CDR3 as the heavy chain CDR3 of CTAT.14 and comprise one or more amino acid variations in one or more of the other heavy chain and light chain CDRs.

In some examples, the anti-TAA binding moiety may comprise the same heavy chain CDRs as those in antibody CTAT.15, which are provided in Table 2 above. Alternatively or in addition, the anti-TAA binding moiety may have the same light chain CDRs as those in antibody CTAT.15, which are also provided in Table 2 above. Such an anti-TAA binding moiety may comprise the same V_{H} and/or V_{L} chains as CTAT.15. Alternatively, the anti-TAA binding moiety may comprise amino acid variations in one or more of the framework regions relative to the corresponding framework regions in CTAT.15. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.15.

In some embodiments, the anti-TAA moiety may comprise a certain level of variations in one or more of the CDRs relative to those of CTAT.15. For example, the anti-TAA moiety may comprise heavy chain CDRs that are at least 80% (e.g., 85%, 90%, 95%, or 98%) sequence identity, individually or collectively, as compared with the V_{H} CDRs of CTAT.15. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.15.

In some instances, the anti-TAA moiety may comprise up to 10 amino acid variations (e.g., up to 9, 8, 7. 6, 5, 4, 3, 2, or l amino acid variations) in one or more of the heavy chain and light chain CDRs collectively relative to those in the CDRs of CTAT.15. In some instances, the anti-TAA moiety may comprise the same heavy chain CDR3 as the heavy chain CDR3 of CTAT.15 and comprise one or more amino acid variations in one or more of the other heavy chain and light chain CDRs.

In some examples, the anti-TAA binding moiety may comprise the same heavy chain CDRs as those in antibody CTAT.16, which are provided in Table 2 above. Alternatively or in addition, the anti-TAA binding moiety may have the same light chain CDRs as those in antibody CTAT.16, which are also provided in Table 2 above. Such an anti-TAA binding moiety may comprise the same V_{H} and/or V_{L} chains as CTAT.16. Alternatively, the anti-TAA binding moiety may comprise amino acid variations in one or more of the framework regions relative to the corresponding framework regions in CTAT.16. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.16.

In some embodiments, the anti-TAA moiety may comprise a certain level of variations in one or more of the CDRs relative to those of CTAT.16. For example, the anti-TAA moiety may comprise heavy chain CDRs that are at least 80% (e.g., 85%, 90%, 95%, or 98%) sequence identity, individually or collectively, as compared with the V_{H} CDRs of CTAT.16. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.16.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.16. In some instances, the anti-TAA moiety may comprise the same heavy chain CDR3 as the heavy chain CDR3 of CTAT.16 and comprise one or more amino acid variations in one or more of the other heavy chain and light chain CDRs.
(iii) Anti-CD3/Anti-TAA Bispecific Antibodies

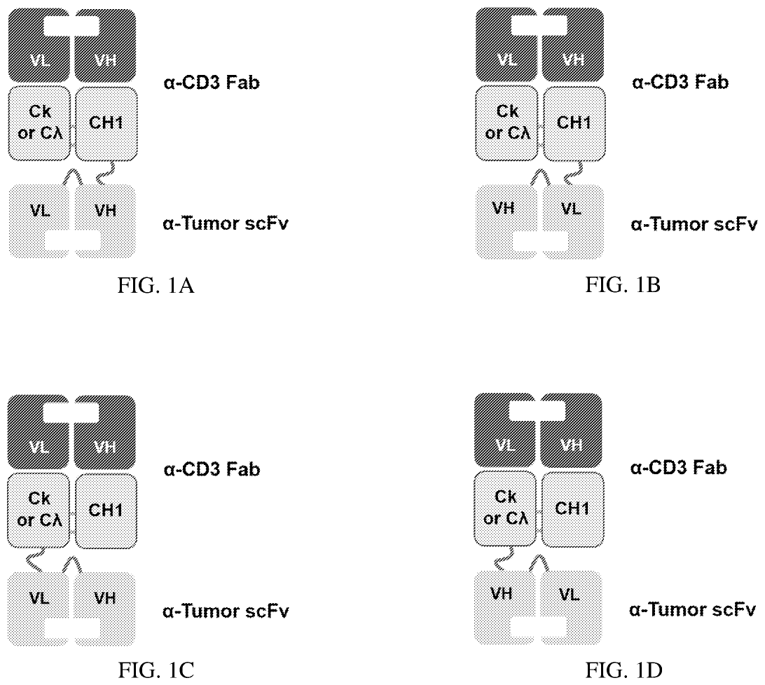

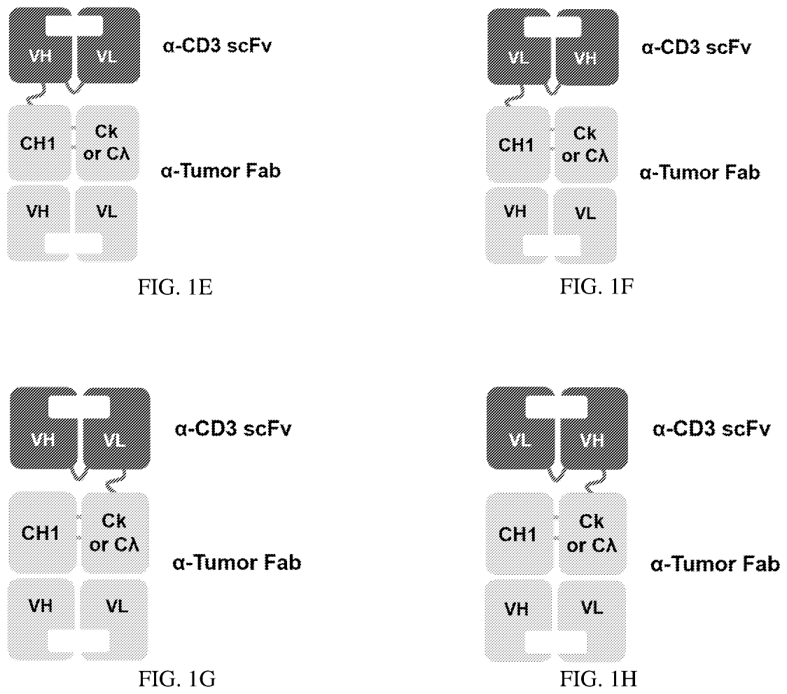

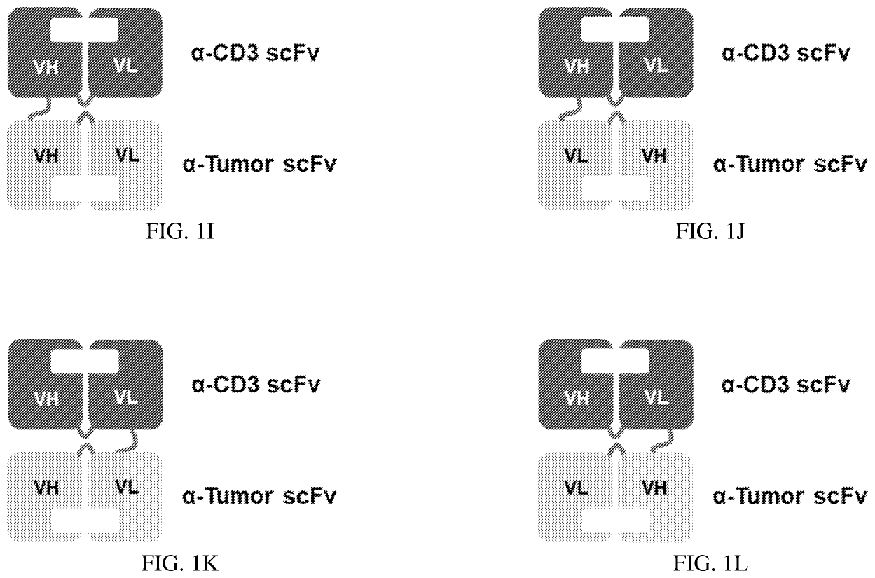

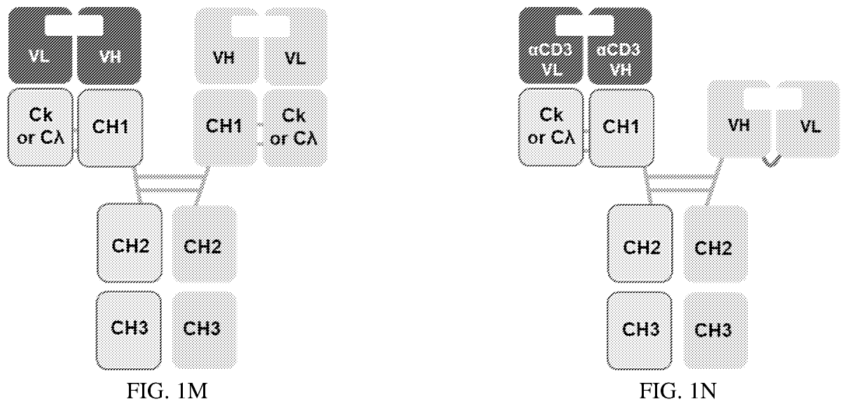

The bispecific antibody disclosed herein may be in any suitable format as those known in the art, for example, those disclosed in Mol. Immunol. 67(2):95-106 (2015), the relevant disclosures of which are incorporated by reference for the subject matter and purpose referenced herein. Some examples are provided below. See also FIGS. 1A-1N.

In some embodiments, the bispecific antibody disclosed herein may comprise one antigen binding moiety in Fab format and the other antigen binding moiety in single chain variable fragment (scFv) format. Such a bispecific antibody may comprise two polypeptides, one comprising the heavy or light chain of the Fab fragment linked to the scFv fragment and the other comprising the light or heavy chain of the Fab that is not linked to the scFv fragment.

In some instances, a Fab fragment comprises two polypeptide chains, one comprising a V_{H} domain linked to a fragment of a heavy chain constant region (e.g., CH1) and the other one comprising a V_{L} domain linked to a light chain constant region. The heavy chain constant region fragment may be from any Ig subclass, for example, IgG, IgA, IgE, IgD, or IgM. In some examples, the heavy chain constant region fragment is from an IgG molecule (e.g., a human IgG molecule). The light chain constant region may be a kappa chain or a lambda chain (e.g., a human kappa or lambda chain). An scFv fragment comprises a V_{H} domain and a V_{L} domain linked by a peptide linker. See e.g., Bird et al. (1988) Science 242:423-426; and

Huston et al. (1988) Proc. Natl. Acad. Sci. USA 85:5879-5883. In some instances, the scFv fragment has, form N-terminus to C-terminus, the VH-linker-V_{L} orientation. Alternatively, the scFv fragment has, form N-terminus to C-terminus, the VL-linker-V_{H} orientation. In the bispecific antibody, the scFv fragment may be linked to the heavy chain of the Fab fragment. Alternatively, the scFv may be linked to the light chain of the Fab fragment. See FIGS. 1A-1H.

In some examples, the bispecific antibody disclosed herein may comprise the anti-CD3 binding moiety in Fab format and the anti-TAA binding moiety in scFv format. Exemplary illustrations are provided in FIGS. 1A-1D. The anti-CD3 Fab comprises a heavy chain VH-CH1 domain and a light chain VL-Cκ or VL-Cλ. domain. The anti-TAA scFv comprises a V_{H} domain and a V_{L} domain. FIGS. 1A-1D. In some instances, the anti-CD3 Fab may be linked to the anti-TAA scFv via a peptide linker disposed between the CH1 domain of the anti-CD3 Fab heavy chain and the V_{H} domain of the anti-tumor scFv. An exemplary illustration is provided in FIG. 1A. In other instances, the CH1 domain of the anti-CD3 Fab heavy chain can be linked to the V_{L} domain of the anti-tumor scFv as illustrated in FIG. 1B. In other instances, the anti-TAA scFv can be linked to the Cκ or Cλ domain of the anti-CD3 Fab light chain via the V_{L} domain of the scFv (FIG. 1C), or via the V_{H} domain of the anti-tumor scFv (FIG. 1D). Examples of anti-CD3 Fab heavy chain (VH-CH1) and light chains (VL-Ck) and examples of anti-TAA scFv fragments are provided in Tables 1 and 2, respectively. Any combination of such is within the scope of the present disclosure.

In some examples, the bispecific antibody disclosed herein may comprise the anti-TAA binding moiety in Fab format and the anti-CD3 binding moiety in scFv format. Exemplary illustrations are provided in FIGS. 1E-1H. The anti-TAA Fab comprises a heavy chain VH-CH1 domain and a light chain VL-Cκ or VL-Cλ domain. The anti-CD3 scFv comprises a V_{H} domain and a V_{L} domain. FIGS. 1E-1H. In some instances, the anti-TAA Fab may be linked to the anti-CD3 scFv via a peptide linker disposed between the CH1 domain of the anti-TAA Fab heavy chain and the V_{H} domain of the anti-CD3 scFv. An exemplary illustration is provided in FIG. 1E. In other instances, the CH1 domain of the anti-TAA Fab heavy chain can be linked to the V_{L} domain of the anti-CD3 scFv as illustrated in FIG. 1F. In other instances, the anti-CD3 scFv can be linked to the Cκ or Cλ domain of the anti-TAA Fab light chain via the V_{L} domain of the scFv (FIG. 1G), or via the V_{H} domain of the anti-CD3 scFv (FIG. 1H). Examples of anti-TAA Fab heavy chain (VH-CH1) and light chains (VL-Ck) and examples of anti-CD3 scFv fragments are provided in Tables 2 and 1, respectively. Any combination of such is within the scope of the present disclosure.

In some embodiments, the bispecific antibody disclosed herein may comprise both antigen binding moieties in scFv format. Exemplary illustrations are provided in FIGS. 1I to 1L.

In some examples, the V_{H} domain of anti-CD3 scFv may be linked to the V_{H} domain of the anti-TAA scFv via a peptide linker (FIG. 1I). In some examples, the V_{H} domain of anti-CD3 scFv may be linked to the V_{L} domain of the anti-TAA scFv via a peptide linker (FIG. 1J). In some examples, the V_{L} domain of anti-CD3 scFv may be linked to the V_{H} domain of the anti-TAA scFv via a peptide linker (FIG. 1K). In other examples, the V_{L} domain of anti-CD3 scFv may be linked to the V_{H} domain of the anti-TAA scFv via a peptide linker (FIG. 1L). Exemplary anti-CD3 scFv fragments and exemplary anti-TAA scFv fragments are provided in Tables 1 and 2, respectively. Any combination thereof for constructing a bispecific antibody is within the scope of the present disclosure.

In yet other embodiments, the bispecific antibodies disclosed herein may comprise one or more Fc regions, which may optionally a “knob into hole” structure, in which a knob in the CH2 domain, the CH3 domain, or both of the first heavy chain is created by replacing several amino acid side chains with alternative ones, and a hole in the juxtaposed position at the CH3 domain of the second heavy chain is created by replacing appropriate amino acid side chains with alternative ones. Exemplary illustrations are provided in FIGS. 1M and 1N.

Typically, the terms “a knob and a hole” or “knobs-into-holes” are used interchangeably herein. Knobs-into-holes amino acid changes is a rational design strategy known in the art for heterodimerization of the heavy (H) chains in the production of bispecific IgG antibodies. Carter, J. Immunol. Methods, 248(1-2):7-15 (2001), the relevant disclosures of which are incorporated by reference herein for the purpose and subject matter referenced herein.

In one example, the “knobs-into-holes” provides an approach as described in, e.g., Ridgway J B B et al., (1996) Protein Engineering, 9(7): 617-21 and U.S. Pat. No. 5,731,168, the relevant disclosures of each of which are incorporated by reference herein for the purpose and subject matter referenced herein. This approach has been shown to promote the formation of heterodimers of the first polypeptide and the second polypeptide chain, and hinder the assembly of corresponding homodimers. In one aspect, a knob is created by replacing small amino side chains at the interface between CH3 domains with larger ones, whereas a hole is constructed by replacing large side chains with smaller ones. In a specific example, the “knob” mutation comprises T366W and the “hole” mutations comprise T366S, L368A and Y407V (Atwell S et al., (1997) J. Mol. Biol. 270: 26-35).

In some instances, the bispecific antibody may comprise an anti-CD3 binding moiety comprising a first VH-CH1-CH2-CH3 domain and a first VL-Cκ or VL-Cλ domain, and an anti-TAA binding moiety comprising a second VH-CH1-CH2-CH3 domain and second a VL-Cκ or VL-Cλ domain. FIG. 1M. The CH2 and/or CH3 in the heavy chain of the anti-CD3 binding moiety that those in the heavy chain of the anti-TAA binding moiety may comprise the knob/hole modifications, allowing for the binding between the two heavy chains. In other instances, the bispecific antibody may comprise an anti-Cd3 binding moiety comprising a first VH-CH1-CH2-CH3 domain and a first VL-Cκ or VL-Cλ domain, and an anti-TAA scFv linked to a second CH2-CH3 domain. The CH2 and/or CH3 in the heavy chain of the anti-CD3 binding moiety that those in the anti-TAA binding moiety may comprise the knob/hole modifications, allowing for the binding between the two heavy chains. FIG. 1N. In this setting, the format of the anti-CD3 binding moiety and the format of the anti-TAA binding moiety may be switched.

The term “peptide linker” refers to a peptide having natural or synthetic amino acid residues for connecting two polypeptides. For example, the peptide linker may be used to connect one V_{H} domain and one V_{L} domain to form a single chain variable fragment (e.g., scFv); to connect one scFv and one Fab to form a scFv/Fab recombinant antibody; to connect two scFvs to form a scFv/scFv recombinant antibody; or to connect two monovalent antibodies (e.g., two monovalent IgGs), two monovalent antibody fragments (e.g., two monovalent scFv-Fc fusion proteins), or one monovalent antibody and one monovalent antibody fragment (e.g., one monovalent IgG and on monovalent scFv-Fc fusion protein) thereby forming a divalent antibody. Preferably, the peptide linker is a peptide having at least 5 amino acid residues in length, such as 5 to 100 amino acid residues in length; more preferably, 10 to 30 amino acid residues in length. The peptide linker within scFv is a peptide of at least 5 amino acid residues in length, preferably 15 to 20 amino acid residues in length. Preferably, the peptide linker comprises a sequence of (GₙS)ₘ, with G=glycine, S=serine, and n and m are independently a number between 1 to 4. In one example, the linker comprises a sequence of (G₂S)₄. In another example, the linker comprises a sequence or (G₄S)₃.

The peptide linker for linking the first antibody fragment (i.e., anti-CD3 antibody fragment) and the second antibody fragment (i.e., anti-TAA antibody fragment) may be any peptide suitable for connecting two polypeptides. According to certain embodiments of the present disclosure, the peptide linker is a peptide having at least 5 amino acid residues in length, for example, having 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more amino acid residues in length. Preferably, the peptide linker of the present recombinant antibody consists of 10 to 30 glycine (G) and/or serine (S) residues.

In some embodiments, the bispecific antibodies described herein specifically bind to one or both of the corresponding target antigen (CD3 and a TAA) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (CD3 and/or a TAA) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, a bispecific antibody as described herein has a suitable binding affinity for one or both of the target antigens (e.g., CD3 and a TAA) or antigenic epitopes thereof. As used herein, “binding affinity” refers to the apparent association constant or KA. The K_{A} is the reciprocal of the dissociation constant (K_{D}). The bispecific antibody described herein may have a binding affinity (K_{D}) of at least 100 nM, 10nM, 1 nM, 0.1 nM, or lower for CD3 (e.g., lower than 1 nM or 0.1 nM). Alternatively, the bispecific antibody described herein may have a binding affinity (K_{D}) of at least 100 nM, 10nM, 1 nM, 0.1 nM, or lower for the TAA.

An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 105 fold. In some embodiments, any of the anti-CD3 and/or anti-TAA antibodies for making the bispecific antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Exemplary bispecific antibodies as disclosed herein are provided in Table 3 below (using anti-CD3 binding moieties from CTA.03 as examples). Anti-CD3 binding moieties from other anti-CD3 reference antibodies (e.g., CTA.02, CTA.04, and CTA.05) are also within the scope of the present disclosure.

+0 TABLE 3
Exemplary Bispecific Antibodies

| Bi-Specific | | |
|---|---|---|
| Antibodies | | Amino Acid Sequences |
| CTA03Fab/ | 1^{st} chain | EVQLLESGGGLVQPGGSLRLSCAASGFTFSSFPMAWVRQAPGKGLEWVSTISTS |
| CTAT01scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLQQPGAELVKPGASVKMSCKASGYTFTSYNMHWVKQTP |
| | | GRGLEWIGAIYPGNGDTSYNQKFKGKATLTADKSSSTAYMQLSSLTSEDSAVYY |
| | | CARSTYYGGDWYFNVWGAGTTVTVSAGGGGSGGGGSGGGGSQIVLSQSPAILSA |
| | | SPGEKVTMTCRASSSVSYIHWFQQKPGSSPKPWIYATSNLASGVPVRFSGSGSG |
| | | TSYSLTISRVEAEDAATYYCQQWTSNPPTFGGGTKLEIK (SEQ ID NO: 229) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT02scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPDGTVKLLIYHTSRLHSGVPSRFSGSGSG |
| | | TDYSLTISNLEQEDIATYFCQQGNTLPYTFGGGTKLEIT (SEQ ID NO: 230) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT03scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLKQSGPGLVQPSQSLSITCTVSGFSLTNYGVHWVRQSP |
| | | GKGLEWLGVIWSGGNTDYNTPFTSRLSINKDNSKSQVFFKMNSLQSNDTAIYYC |
| | | ARALTYYDYEFAYWGQGTLVTVSAGGGGSGGGGSGGGGSDILLTQSPVILSVSP |
| | | GERVSFSCRASQSIGTNIHWYQQRINGSPRLLIKYASESISGIPSRFSGSGSGT |
| | | DFTLSINSVESEDIADYYCQQNNNWPTTFGAGTKLELK (SEQ ID NO: 231) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT04scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVESGGGLVQPGGSLRLSCAASGFNIKDTYIHWVRQAP |
| | | GKGLEWVARIYPTNGYTRYADSVKGRFTISADTSKNTAYLQMNSLRAEDTAVYY |
| | | CSRWGGDGFYAMDYWGQGTLVTVSSGGGGSGGGGSGGGGSDIQMTQSPSSLSAS |
| | | VGDRVTITCRASQDVNTAVAWYQQKPGKAPKLLIYSASFLYSGVPSRFSGSRSG |
| | | TDFTLTISSLQPEDFATYYCQQHYTTPPTFGQGTKVEIK (SEQ ID NO: 232) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT05scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVQSGAEVKKPGASVKISCKTSGYTFTEYTIHWVKQAS |
| | | GKGLEWIGNINPNNGGTTYNQKFEDRATLTVDKSTSTAYMELSSLRSEDTAVYY |
| | | CAAGWNFDYWGQGTTVTVSSGGGGSGGGGSGGGGSDIVMTQSPSSLSASVGDRV |
| | | TITCKASQDVGTAVDWYQQKPGKAPKLLIYWASTRHTGVPDRFTGSGSGTDFTL |
| | | TISSLQPEDFADYFCQQYNSYPLTFGGGTKLEIK (SEQ ID NO: 233) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT06scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLVQSGSELKKPGASVKVSCKASGYTFTEYGMNVWRQAP |
| | | GQGLEWMGWINTKSGEATYVEEFKGRFVFSLDTSVSTAYLQISSLKAEDTAVYY |
| | | CARWDFYDYVDEAMYWGQGTTVTVSSGGGGSGGGGSGGGGSDIQMTQSPSSLSA |
| | | SVGDRVTITCKASQTVSANVAWYQQKPGKAPKLLIYLASYRYRGVPSRFSGSGS |
| | | GTDFTLTISSLQPEDFATYYCHQYYTYPLFTFGQGTKLEIK (SEQ ID NO: |
| | | 234) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT07scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVQSGPGLVQPGGSVRISCAASGYTFTNYGMNWVKQAP |
| | | GKGLEWMGWINTYTGESTYADSFKGRFTFSLDTSASAAYLQINSLRAEDTAVYY |
| | | CARFAIKGDYWGQGTLLTVSSGGGGSGGGGSGGGGSDIQMTQSPSSLSASVGDR |
| | | VTITCRSTKSLLHSNGITYLYWYQQKPGKAPKLLIYQMSNLASGVPSRFSSSGS |
| | | GTDFTLTISSLQPEDFATYYCAQNLEIPRTFGQGTKVELK (SEQ ID NO: |
| | | 235) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT08scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLVQSGAEVKKPGASVKVSCKTSRYTFTEYTIHWVRQAP |
| | | GQRLEWIGGINPNNGIPNYNQKFKGRVTITVDTSASTAYMELSSLRSEDTAVYY |
| | | CARRRIAYGYDEGHAMDYWGQGTLVTVSSGGGGSGGGGSGGGGSDIVMTQSPDS |
| | | LAVSLGERATINCKSSQSLLYSRNQKNYLAWYQQKPGQPPKLLIFWASTRESGV |
| | | PDRFSGSGFGTDFTLTISSLQAEDVAVYYCQQYFSYPLTFGQGTKVEIK |
| | | (SEQ ID NO: 236) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT09scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVESGGGLVQPGGSLRLSCAASGFTFSDSWIHWVRQAP |
| | | GKGLEWVAWISPYGGSTYYADSVKGRFTISADTSKNTAYLQMNSLRAEDTAVYY |
| | | CARRHWPGGFDYWGQGTLVTVSSGGGGSGGGGSGGGGSDIQMTQSPSSLSASVG |
| | | DRVTITCRASQDVSTAVAWYQQKPGKAPKLLIYSASFLYSGVPSRFSGSGSGTD |
| | | FTLTISSLQPEDFATYYCQQYLYHPATFGQGTKVEIK (SEQ ID NO: 237) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT10scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLLESGGGLVQPGGSLRLSCAVSGFTFNSFAMSWVRQAP |
| | | GKGLEWVSAISGSGGGTYYADSVKGRFTISRDNSKNTLYLQMNSLRAEDTAVYF |
| | | CAKDKILWFGEPVFDYWGQGTLVTVSSGGGGSGGGGSGGGGSEIVLTQSPATLS |
| | | LSPGERATLSCRASQSVSSYLAWYQQKPGQAPRLLIYDASNRATGIPARFSGSG |
| | | SGTDFTLTISSLEPEDFAVYYCQQRSNWPPTFGQGTKVEIK (SEQ ID NO: |
| | | 238) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT11scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVQSGAEVKKPGSSVKVSCKASGYTITDSNIHWVRQAP |
| | | GQSLEWIGYIYPYNGGTDYNQKFKNRATLTVDNPTNTAYMELSSLRSEDTAFYY |
| | | CVNGNPWLAYWGQGTLVTVSSGGGGSGGGGSGGGGSDIQLTQSPSTLSASVGDR |
| | | VTITCRASESLDNYGIRFLTWFQQKPGKAPKLLMYAASNQGSGVPSRFSGSGSG |
| | | TEFTLTISSLQPDDFATYYCQQTKEVPWSFGQGTKVEVK (SEQ ID NO: 239) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT12scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVESGGGLVQPGGSLRLSCAASGYTFTSYWLHWVRQAP |
| | | GKGLEWVGMIDPSNSDTRFNPNFKDRFTISADTSKNTAYLQMNSLRAEDTAVYY |
| | | CATYRSYVTPLDYWGQGTLVTVSSGGGGSGGGGSGGGGSDIQMTQSPSSLSASV |
| | | GDRVTITCKSSQSLLYTSSQKNYLAWYQQKPGKAPKLLIYWASTRESGVPSRFS |
| | | GSGSGTDFTLTISSLQPEDFATYYCQQYYAYPWTFGQGTKVEIK (SEQ ID NO: |
| | | 240) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT13scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLVQSGAEVKKPGASVKVSCKASGYTFTNYNMHWVRQAP |
| | | GQRLEWMGTIYPGNDDTSYNQKFKDRVTITADTSASTAYMELSSLRSEDTAVYY |
| | | CARGGYRAMDYWGQGTLVTVSSGGGGSGGGGSGGGGSDIVMTQSPLSLPVTPGE |
| | | PASISCRSSQSIVYSNGNTYLGWYLQKPGQSPQLLIYKVSNRFSGVPDRESGSG |
| | | SGTDFTLKISRVEAEDVGVYYCFQGSHVPYTFGQGTKLEIK (SEQ ID NO: |
| | | 241) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT14scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLQESGPGLVKPSQTLSLTCTVSGGSISSGDYFWSWIRQ |
| | | LPGKGLEWIGHIHNSGTTYYNPSLKSRVTISVDTSKKQFSLRLSSVTAADTAVY |
| | | YCARDRGGDYYYGMDVWGQGTTVTVSSGGGGSGGGGSGGGGSEIVLTQSPGTLS |
| | | LSPGERATLSCRASQGISRSYLAWYQQKPGQAPSLLIYGASSRATGIPDRFSGS |
| | | GSGTDFTLTISRLEPEDFAVYYCQQFGSSPWTFGQGTKVEIK (SEQ ID NO: |
| | | 242) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT015scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLQESGPGLVKPSETLSLTCTVSGGSVSSGSYYWSWIRQ |
| | | PPGKGLEWIGYIYYSGSTNYNPSLKSRVTISVDTSKNQFSLKLSSVTAADTAVY |
| | | YCAREGKNGAFDIWGQGTMVTVSSGGGGSGGGGSGGGGSDIQMTQSPSSLSASV |
| | | GDRVTITCRASQSISSYLNWYQQKPGKAPKLLIYAASSLQSGVPSGFSGSGSGT |
| | | DFTLTISSLQPEDFATYYCQQSYSTPLTFGGGTKVEIK (SEQ ID NO: 243) |
| | 2^{nd} chain | CTA.03 VL-Ck (SEQ ID NO: 24) |
| CTA03Fab/ | 1^{st} chain | EVQLLESGGGLVQPGGSLRLSCAASGFTFSSFPMAWVRQAPGKGLEWVSTISTS |
| CTAT16scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLLQSGPELEKPGASVMISCKASGSSFTGYNMNWVRQNI |
| | | GKSLEWIGAIDPYYGGTSYNQKFKGRATLTVDKSSSTAYMHLKSLTSEDSAVYY |
| | | CVSGMEYWGQGTSVTVSSGGGGSGGGGSGGGGSEIVMTQSPATLSVSPGERATL |
| | | SCRSSQSLVHRNGNTYLHWYLQKPGQSPKLLIHKVSNRFSGVPDRFSGSGSGTD |
| | | FTLKISRVEAEDLGVYFCSQSTHVPPLTFGAGTKLELK (SEQ ID NO: 244) |
| | 2^{nd} chain | CTA.03 VL-Ck (SEQ ID NO: 24) |
| CTA03-01Fab/ | 1^{st} chain | EVQLLESGGGLVQPGGSLRLSCAASGFTFSSFPMAWVRQAPGKGLEWVSTISTS |
| CTAT02-01scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPDATVKLLIYHTSRLHSGVPSRFSGSGSG |
| | | TDYSLTISNLEQEDIATYFCQQGNTLPYTFGGGTKLEIT (SEQ ID NO: 245) |
| | 2^{nd} chain | CTA.03 VL-Ck-01 (SEQ ID NO: 228) |
| CTA03-01Fab/ | 1^{st} chain | EVQLLESGGGLVQPGGSLRLSCAASGFTFSSFPMAWVRQAPGKGLEWVSTISTS |
| CTAT02-02scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPEGTVKLLIYHTSRLHSGVPSRFSGSGSG |
| | | TDYSLTISNLEQEDIATYFCQQGNTLPYTFGGGTKLEIT (SEQ ID NO: 246) |
| | 2^{nd} chain | CTA.03 VL-Ck-01 (SEQ ID NO: 228) |
| CTA03-02Fab/ | 1^{st} chain | EVQLLESGGGLVQPGGSLRLSCAASGFTFSSFPMAWVRQAPGKGLEWVSTISTS |
| CTAT02-02scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPEGTVKLLIYHTSRLHSGVPSRFSGSGSG |
| | | TDYSLTISNLEQEDIATYFCQQGNTLPYTFGGGTKLEIT (SEQ ID NO: 247) |
| | 2^{nd} chain | CTA.03 VL-Ck-02 (SEQ ID NO: 25) |
| CTA03-02Fab/ | 1^{st} chain | EVQLLESGGGLVQPGGSLRLSCAASGFTFSSFPMAWVRQAPGKGLEWVSTISTS |
| CTAT02-01scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPDATVKLLIYHTSRLHSGVPSRFSGSGSG |
| | | TDYSLTISNLEQEDIATYFCQQGNTLPYTFGGGTKLEIT (SEQ ID NO: 248) |
| | 2^{nd} chain | CTA.03 VL-Ck-01 (SEQ ID NO: 25) |

Also provided herein are pharmaceutical compositions comprising any of the bispecific antibodies disclosed herein (or the armed immune cells also disclosed herein), which further comprises a pharmaceutically acceptable excipient. The pharmaceutically acceptable excipient may be any inert substance that is combined with an active molecule (such as the bispecific antibody or the armed immune cells) for preparing an agreeable or convenient dosage form. In general, the pharmaceutically acceptable excipient is non-toxic to recipients at the dosages and concentrations employed, and is compatible with other ingredients of the formulation comprising the recombinant antibody. Examples of the pharmaceutically acceptable excipient suitable to be employed in the present pharmaceutical composition include, but are not limited to, water, phosphate buffer, acetate buffer, succinate buffer, citrate buffer, tris(hydroxymethyl)aminomethane (Tris) buffer, phosphate-buffered saline (PBS), Ringer's solution, lactated Ringer's solution, and a combination thereof. Optionally, the pharmaceutical composition may further comprise an agent for storing and/or stabilizing the recombinant antibody, e.g., amino acid reside (such as, histidine (H) or serine (S) residue), glucose, galactose, xylitol, sorbitol, mannitol, sucrose, trehalose, or antioxidant. Other agents may also be added, such as antimicrobial agents, to prevent spoilage upon storage, i.e., to inhibit growth of microbes such as yeasts and molds.
B. Methods for Producing Bispecific Antibodies

Any of the bispecific antibodies described herein can be made by any method known in the art. See, for example, Harlow and Lane, (1998) Antibodies: A Laboratory Manual, Cold Spring Harbor Laboratory, New York. In some embodiments, the anti-CD3 antibody and/or the anti-TAA antibody for use in making the bispecific antibodies may be produced by the conventional hybridoma technology. Alternatively, the anti-CD3 and/or anti-TAA antibody may be identified from a suitable library (e.g., a human antibody library). In some instances, high affinity fully human CD3 and/or TAA binders may be obtained from a human antibody library, for example, affinity maturation libraries (e.g., having variations in one or more of the CDR regions). There are a number of routine methods known in the art to identify and isolate antibodies capable of binding to the target antigens described herein, including phage display, yeast display, ribosomal display, or mammalian display technology.

In some embodiments, the bispecific antibodies disclosed herein may be produced by the conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide.

In some instances, nucleic acids encoding the one or both chains of a bispecific antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and Co1E1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

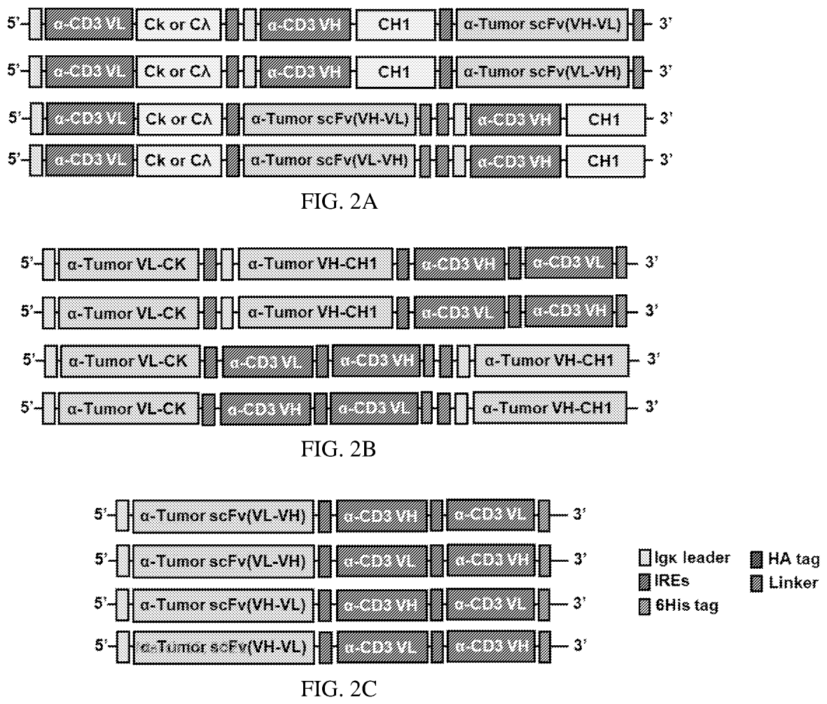

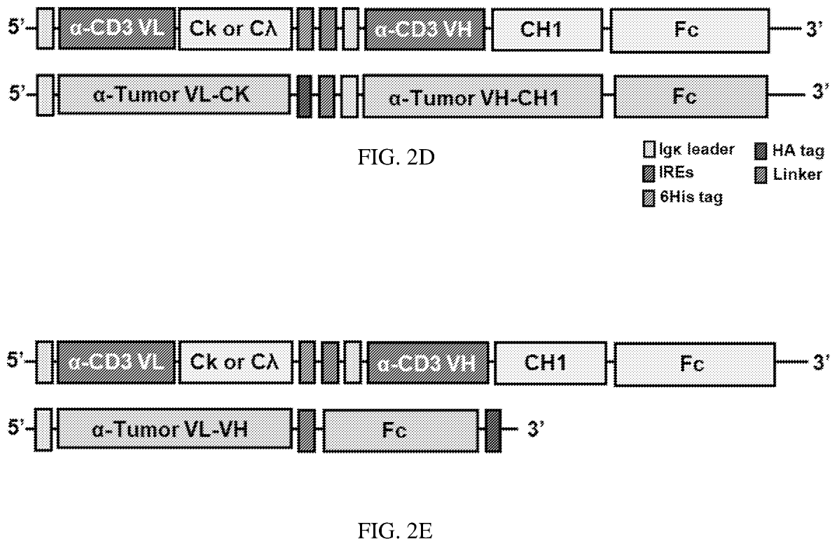

Exemplary constructs for producing the bispecific antibodies in various configuration as disclosed herein are provided in FIGS. 2A-2E.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both chains of a bispecific antibody as described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, each encoding one chain of a bispecific antibody disclosed herein. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the bispecific antibody as described herein, vectors (e.g., expression vectors) containing such; and host cells comprising the vectors are within the scope of the present disclosure. Methods for producing such bispecific antibodies (e.g., using host cells via the recombinant technology) are also within the scope of the present disclosure.

### II. Armed Immune Cells

In another aspects, provided herein are immune cells armed with any of the bispecific antibodies disclosed herein (e.g., those comprising Fab fragment and/or scFv chains provided in Tables 1 and 2, or the exemplary bispecific antibodies provided in Table 3 above). The bispecific antibody can be displayed on the surface of CD3+ immune cells via binding to the cell surface CD3 molecule.

The immune cells may be any type of immune cells (e.g., human immune cells) expressing surface CD3 or a mixture thereof. Examples include, but are not limited to, T cell, a B cell, a monocyte, and/or macrophage. In some instances, the T cell is a traditional CD4+ and/or CD8+ T cells. In some instances, the T cell is a regulatory T cell (Treg). In other instances, the T cell is a natural killer T cell (NKT). The immune cells may be obtained from a donor such as a humor donor (e.g., a healthy donor). Alternatively, the immune cells may be obtained from a cell line or differentiated from stem cells, for example, hematopoietic stem cells, bone marrow cells, umbilical cord blood cells, or induced pluripotent stem cells.

Any of the armed immune cells may be produced by incubating suitable immune cells with any of the bispecific antibodies disclosed herein (e.g., those comprising Fab fragment and/or scFv chains provided in Tables 1 and 2, or the exemplary bispecific antibodies provided in Table 3 above) under suitable conditions for a suitable period of time. Unlike anti-CD3 antibody alone (e.g., OKT3), incubation of the bispecific antibodies disclosed herein with immune cells result in production of armed immune cells having the bispecific antibody displayed on the cell surface. The bispecific antibody may induce proliferation and/or differentiation of immune cells, such as induce differentiation of naïve T cells into effector cells via binding to the CD3 molecule on T cells via its anti-CD3 binding moiety. The armed immune cells thus produced is capable of targeting cancer cells via recognizing the TAA molecule expressed on the cancer cells by the anti-TAA binding moiety of the bispecific antibody, which is displayed on the surface of the armed immune cells.

In some examples, the armed immune cells disclosed herein can be produced using peripheral blood mononuclear cells (PBMCs). For example, PBMCs can be isolated from a donor (e.g., a human donor) using a conventional method. The methods suitable for isolating PBMCs from a donor include, but are not limited to, density centrifugation (e.g., FICOLL® Paque), cell preparation tube (CPT), and SEPMATE™ tube. In some examples, the PBMCs can be isolated from a whole blood sample obtained from a donor via density centrifugation according to the manufacturer's directions. The isolated PBMCs can then be cultivated with the bispecific antibody in a suitable cell culture medium for least 7 days, such as 7, 8, 9, 10, 11, 12, 13 14, or more days; preferably, for at least 14 days. In some embodiments, the number of CD3⁺ immune cells such as T cells (e.g., CD4/CD8 T cells and/or NKT cells) multiplies after cultivation for 7 days. In other embodiments, cultivation is continued for 14 days, and the number of CD3⁺ T cells increases for 3 folds.

In other examples, immune cells from cell culture may be used for making the armed immune cells disclosed herein. The in vitro cultured immune cells may be from an established cell line. Alternatively, the immune cells may be differentiated from suitable stem cells, for example, hematopoietic stem cells, bone marrow cells, umbilical cord blood cells, or induced pluripotent stem cells, following conventional methods.

A suitable amount of immune cells (e.g., 3×10⁵ cells) may be cultured in a suitable cell culture medium in the presence of about 500 ng to about 3,000 ng (e.g., 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, or 3,000 ng) of a bispecific antibody for a suitable period of time under suitable conditions to produce the armed immune cells. The cell culture medium may comprise one or more cytokines for sustaining the growth of immune cells such as T cells and/or stimulating the activation of the immune cells. Examples include, but are not limited to, IL-1β, IL-2, IL-4, IL-6, IL-7, IL-12, IL-18, IL-21, IL-23, IL-25, IL-27, IL-31, interferon-gamma (IFN-γ), TGF-β, or a combination thereof. Additionally or alternatively, the medium may comprise an antibody or a carbohydrate for the activation purpose, such as an anti-CD28 antibody or a mannose.

In some examples, IL-2 may be used in the culture medium to cultivate PBMCs to produce, e.g., armed CD8⁺ T cells. In other examples, IL-2 and IL-7 may be used in the culture medium to cultivating PBMCs for producing, e.g., armed CD4+ T cells. For producing armed Treg cells, IL-2, an anti-CD28 antibody, and mannose may be used in the cell culture medium.

The armed immune cells produced by any of the methods disclosed herein are also within the scope of the present disclosure.

### III. Cancer Treatment With Armed Immune Cells

In another aspect, the present disclosure provides a method for treating cancer using the armed immune cells disclosed herein. To practice the method disclosed herein, an effective amount of the armed immune cells or a pharmaceutical composition comprising such can be administered to a subject (e.g., a human) in need of the treatment via a suitable route, such as intravenous administration, e.g., as a bolus or by continuous infusion over a period of time. In some instances, the armed immune cells are autologous to the subject. In other instances, the armed immune cells are allogenic to the subject.

The subject to be treated by the methods described herein can be a mammal, more preferably a human or a non-human primate. Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying tumor cells expressing the target TAA, to which a bispecific antibody binds. Exemplary cancers include, but are not limited to, melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, leukemia, lymphoma, and myeloma.

Presence of specific tumor associated antigens by specific types of cancer cells are known in the art. For example, B-cell malignancies often involve CD19+ (e.g., B-cell acute lymphoblastic leukemia) and/or CD20+ cancer cells (e.g., B-cell Non-Hodgkin's lymphoma). EGFR is expressed on various types of cancer, such as lung cancer and colon cancer. HER2 is associated with, for example, breast cancer. PSMA is associated, for example, prostate cancer. CEA is associated with various types of cancer, including colon, rectum, and pancreatic cancer. EpCAM, FAP, CD47, and TRAIL-R2 are associated with solid tumors. PDL1 is associated with various cancers, such as bladder cancer, non-small cell lung cancer, breast cancer, small cell lung cancer, etc. CD38 is associated with, for example, multiple myeloma. CD33 is associated with, for example, AML. cMET (HGFR) is associated, for example, non-small cell lung cancer. Mesothelin is associated with mesothelioma. GD2 is associated with neuroblastoma. Accordingly, choosing a bispecific antibody disclosed herein that has a suitable anti-TAA binding moiety to treat a particular type of cancer is within the knowledge of a medical practitioner.

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery. A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of armed immune cells as described herein will depend on the specific bispecific antibody on the immune cells, the type of immune cells (or compositions thereof) employed, the type and severity of the disease/disorder, the patient's clinical history and response to the agonist, and the discretion of the attending physician. Typically the clinician will administer armed immune cells, until a dosage is reached that achieves the desired result. Methods of determining whether a dosage resulted in the desired result would be evident to one of skill in the art. Administration of one or more doses of armed immune cells can be continuous or intermittent, depending, for example, upon the recipient's physiological condition, whether the purpose of the administration is therapeutic or prophylactic, and other factors known to skilled practitioners. The administration of the armed immune cells may be essentially continuous over a preselected period of time or may be in a series of spaced dose, e.g., either before, during, or after developing a target disease or disorder.

In some embodiments, the amount of the armed immune cells such as armed T cells administered to the subject can be about 1×10⁴ to 1×10⁷ cells/Kg body weight of the subject. In certain embodiments, the amount of armed immune cells such as armed T cells can be administered to the subject from about 1×10⁵ to 1×10⁶ cells/Kg body weight of the subject. The dose can be administered in a single dose, or alternatively in more than one dose.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the armed immune cells or a pharmaceutical composition comprising such to a subject, depending upon the type of cancer to be treated or the site of the cancer. In some instances, the armed immune cells can be administered via intravenous infusion.

In some embodiments, the armed immune cells disclosed herein may be co-used with another anti-cancer agent, for example, a chemotherapeutic agent, an immunotherapeutic agent, or a combination thereof. For example, the armed immune cells disclosed herein may be used in combination with an immune checkpoint inhibitor, such as an anti-PD-1 antibody or an anti-PDLI antibody. As used herein, the term “combination,” “combined,” and related terms refers to the simultaneous or sequential administration of multiple therapeutic agents in accordance with this disclosure. For example, the armed immune cells as disclosed herein may be administered with another therapeutic agent simultaneously or sequentially in separate unit dosage forms or together in a single unit dosage form.

In one example, a method for treating a subject (e.g., a human cancer patient) having cancer cells expressing a TAA using armed immune cells disclosed herein may comprising the following steps: (a) isolating PBMCs from the patient; (b) cultivating the PBMCs of step (a) with a bispecific antibodies disclosed herein, which comprises a binding moiety specific to the TAA so as to produce armed immune cells such as armed T cells; and (c) administering to the subject an effective amount of the armed immune cells.

In the step (a), the PBMCs can be isolated from the subject. The subject may be any mammal, for example, a human, mouse, rat, chimpanzee, rabbit, monkey, sheep, goat, cat, dog, horse, or pig. Preferably, the subject is a human. The methods suitable for isolating PBMCs from the subject include, but are not limited to, density centrifugation (e.g., FICOLL® Paque), cell preparation tube (CPT), and SEPMATE™ tube.

In the step (b), the isolated PBMCs are cultured in a medium containing the present recombinant antibody for a sufficient period of time (e.g., at least 7 days) so as to produce the TAA-specific T cells. The bispecific antibody is capable of inducing the activation of T cells by its anti-CD3 antibody fragment. The thus-produced armed T cells has the bispecific antibody bound on the surface thereof, and accordingly, may specifically target the cancer cells via the anti-TAA antibody fragment of the bispecific antibody.

In the step (c), the armed immune cells such as armed T cells produced in step (b) can be administered to the subject so as to treat cancer. The amount of T cells administered to the subject is from about 1×10⁴ to 1×10⁷ cells/Kg body weight of the subject. In certain embodiments, the amount of T cells is administered to the subject from about 1×10⁵ to 1×10⁶ cells/Kg body weight of the subject. The dose can be administered in a single dose, or alternatively in more than one dose.

Optionally, prior to step (c), the method may further isolating the T cell from the product of step (b) by a method suitable for isolating or purifying immune cells, for example, affinity column, or magnetic beads. Treatment efficacy may be examined via routine practice.

### IV. Kits For Cancer Treatment

The present disclosure also provides kits comprising any of the armed immune cells such as armed T cells or any of the bispecific antibodies disclosed herein. Such kits can be used for treating or alleviating a target cancer as disclosed herein. Such kits can include one or more containers comprising the armed immune cells or a bispecific antibody as those described herein.

In some embodiments, the kit can comprise instructions for use in accordance with any of the methods described herein. The included instructions can comprise a description of administration of the armed immune cells or use of the bispecific antibody to produce the armed immune cells, to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of the armed immune cells such as armed T cells or the bispecific antibody generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is armed immune cells or a bispecific antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Production of Recombinant Bi-Specific Antibodies

In this example, recombinant antibodies respectively having the structures as depicted in FIGS. 1A-1L were prepared using the DNA constructs illustrated in FIGS. 2A-2E. For anti-CD3 Fab/anti-TAA scFv bi-specific antibodies, the constructs comprise, from N-terminus to C-terminus, (a) Igk leader sequence (LS), anti-CD3 VL-Ck domain or anti-CD3 VL-Cλ domain, internal ribosomal entry site (IRES), LS, anti-CD3 VH-CH1 domain, peptide linker, and anti-TAA scFv (e.g., anti-EGFR scFv) (FIGS. 1A-1D) and FIG. 2A, top two constructs; or (b) LS, anti-CD3 VL-Ck domain or anti-CD3 VL-Cλ domain, peptide linker, anti-TAA scFv (e.g., anti-EGFR scFv), IRES, LS, and anti-CD3 VH-CH1 domain (FIG. 1B and FIG. 2A, bottom two constructs.

For anti-CD3 scFv/anti-TAA Fab, the constructs comprise, from N-terminus to C-terminus, (a) LS, anti-TAA VL-Ck domain (e.g., anti-EGFR VL-Ck domain), IRES, LS, anti-TAA VH-CH1 domain (e.g., anti-EGFR VH-CH1 domain), peptide linker, and anti-CD3 VH-V_{L} domain or anti-CD3 VL-V_{H} domain (FIGS. 1E-1H and FIG. 2B, top two constructs); or (b) LS, anti-TAA VL-Ck domain (e.g., anti-EGFR VL-Ck domain), peptide linker, anti-CD3 VH-V_{L} domain or anti-CD3 VL-V_{H} domain, IRES, LS, and anti-TAA VH-CH1 domain (e.g., anti-EGFR VH-CH1 domain) (FIG. 1F and FIG. 2B, bottom two constructs).

For anti-CD3 scFv/anti-TAA scFv, the constructs comprise, from N-terminus to C-terminus, LS, anti-TAA scFv (e.g., anti-EGFR scFv), peptide linker, and anti-CD3 VH-V_{L} domain or anti-CD3 VL-V_{H} domain (FIGS. 1I-1L and FIG. 2C).

For anti-CD3 knob/anti-TAA hole antibody, the anti-CD3 knob constructs comprise, from N-terminus to C-terminus, LS, anti-CD3 VL-Ck domain or anti-CD3 VL-Cλ domain, IRES, LS, and anti-CD3 VH-CH1-knob Fc, while the anti-tumor hole comprised in sequence, LS, anti-TAA VL-Ck (e.g., anti-EGFR VL-Ck domain), IRES, LS, and anti-TAA VH-CH1-hole Fc (e.g., anti-EGFR VH-CH1-hole Fc) (FIG. 2D).

For anti-CD3 knob/anti-TAA scFv hole antibody, the anti-CD3 knob construct comprised in sequence, LS, anti-CD3 VL-Ck domain or anti-CD3 VL-Cλ domain, IRES, LS, and anti-CD3 VH-CH1-knob Fc, while the anti-tumor hole comprised in sequence, LS, anti-TAA scFv (e.g., anti-EGFR scFv), peptide linker, and hole Fc (FIG. 2E).

Two recombinant antibodies, respectively designated as CTA02scFv/CTAT03Fab (previously named anti-EGFR Fab/CAT.02 scFv) and CTA01scFv/CTAT03Fab (previously named anti-EGFR Fab/aCD3 scFv, structural information of which is provided in WO2018177371, the relevant disclosures of which is incorporated by reference for the subject matter and purpose referenced herein), were accordingly prepared. Both the CTA02scFv/CTAT03Fab and CTA01scFv/CTAT03Fab comprised an anti-EGFR Fab and an anti-CD3 scFv, in which the VH-CH1 and VL-Ck domains of the anti-EGFR Fab respectively had the amino acid sequences of SEQ ID NOs: 83 and 84. The anti-CD3 scFv of the CTA02scFv/CTAT03Fab had the amino acid sequence of SEQ ID NO: 9, and the anti-CD3 scFv of the CTA01scFv/CTAT03Fab is provided in WO2018177371 (CTA01 is the same antibody named as OKT3 in WO2018177371).

### Example 2: Effect of Recombinant Bi-Specific Antibodies on T Cells

This example investigates bioactivities of the recombinant bi-specific antibodies disclosed herein.
Binding Affinity

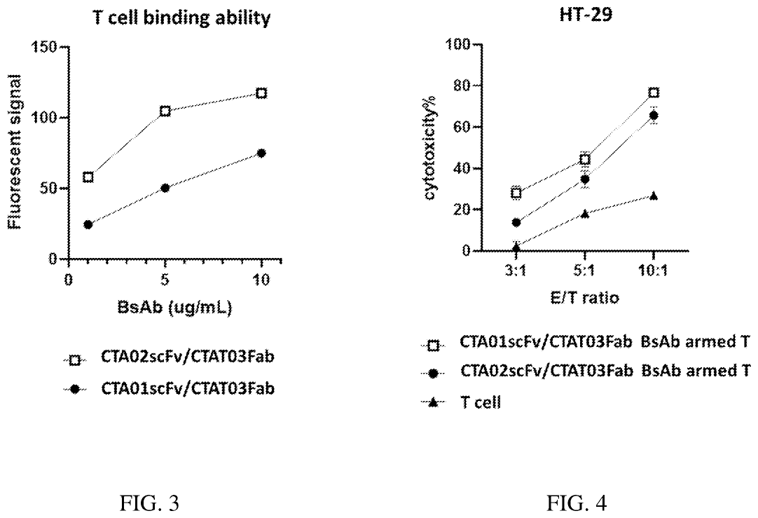

The binding affinities of the recombinant antibodies CTA02scFv/CTAT03Fab and CTA01scFv/CTAT03Fab were examined in this example by flow cytometry. Both the CTA02scFv/CTAT03Fab and the CTA01scFv/CTAT03Fab were capable of binding to CD3-positive T cells. The binding affinity of the CTA02scFv/CTAT03Fab was higher than that of the CTA01scFv/CTAT03Fab. FIG. 3. The binding affinity results are provided in Table 4 below.

TABLE 4
Binding Affinity of Exemplary Bi-Specific Antibodies

| Group | Fluorescent signal |
|---|---|
| T cell | 0 |
| T cell + 1 ug/mL CTA01scFv/CTAT03Fab | 24.44 |
| T cell + 5 ug/mL CTA01scFv/CTAT03Fab | 50.31 |
| T cell + 10 ug/mL CTA01scFv/CTAT03Fab | 74.97 |
| T cell + 1 ug/mL CTA02scFv/CTAT03Fab | 57.95 |
| T cell + 5 ug/mL CTA02scFv/CTAT03Fab | 104.63 |
| T cell + 10 ug/mL CTA02scFv/CTAT03Fab | 117.25 |

Cytotoxic Effect

The cytotoxic effect of the recombinant antibody CTA02scFv/CTAT03Fab or the CTA01scFv/CTAT03Fab on cancer cells was evaluated in this example. It was found that about 2.5%, 18.2% and 26.9% of HT-29 cells were killed by CD3⁺/CD8⁺ T cells activated by murine OKT3 at the effect cells to target cells ratio (E/T ratio) of 3:1, 5:1 and 10:1, respectively; about 13.8%, 34.8% and 65.7% of HT-29 cells were killed by CD3⁺/CD8⁺ T cells armed with the CTA01scFv/CTAT03Fab, and about 28.1%, 44.4% and 76.7% of HT-29 cells were killed by T cells armed with the CTA02scFv/CTAT03Fab at the same E/T ratio (FIG. 4 and Table 5).

TABLE 5
Cytotoxic effect of specified antibodies

| | OKT3 | | |
|---|---|---|---|
| E/T | activated T | CTA01scFv/CTAT03Fab | CTA02scFv/CTAT03Fab |
| ratio | cells | armed T cells | armed T cells |
| 3:1 | 2.5 ± 1.8 | 13.8 ± 0.7 | 28.1 ± 2.7 |
| 5:1 | 18.2 ± 1.5 | 34.8 ± 3.3 | 44.4 ± 3 |
| 10:1 | 26.9 ± 0.6 | 65.7 ± 3.3 | 76.7 ± 1.3 |

The data indicated that both the tested bi-specific antibodies showed higher cytotoxic effects against cancer cells relative to the OKT3 antibody (anti-CD3 antibody) and the CTA02scFv/CTAT03Fab showed better cytotoxicity effect.
Time Effects on the Amounts of Antibodies Remained on the Surfaces of T Cells

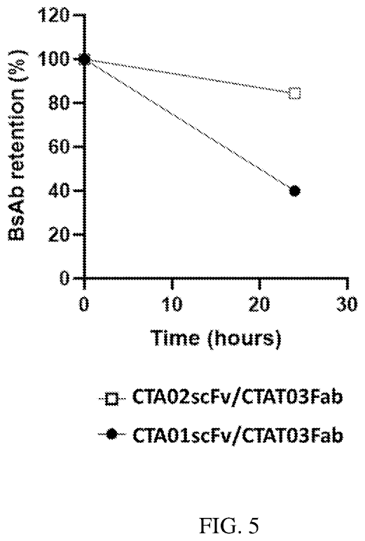

The CTA02scFv/CTAT03Fab and the CTA01scFv/CTAT03Fab were respectively incubated with T cells in the presence of 20% FBS for 24 hours. The T cells were then analyzed by flow cytometry to evaluate the residual amounts of the antibodies on the surface of T cells. The results from this assay indicate that the amounts of the antibodies on the surface of the T cells declined over time. FIG. 5 and Table 6 below. Compared to the CTA01scFv/CTAT03Fab, the CTA02scFv/CTAT03Fab was less affected by degradation. About 84.5% of the CTA02scFv/CTAT03Fab still remained on the T cell surface after 24 hours, while the level of the CTA01scFv/CTAT03Fab remained on the T cell surface dropped to about 40% after 24 hours.

TABLE 6
Percentage of Bi-Specific Antibodies
Remaining On Cell Surface Over Time

| Time (hour) | CTA02scFv/CTAT03Fab | CTA01scFv/CTAT03Fab |
|---|---|---|
| 0 | 100 | 100 |
| 24 | 84.5 ± 0.6 | 40 ± 1.4 |

In sum, the bi-specific antibodies disclosed herein (exemplified by the CTA02scFv/CTAT03Fab antibody) exhibited higher binding affinity to T cells, higher cytotoxicity, and higher level of T cell engagement over time relative to the CTA01scFv/CTAT03Fab control antibody.

### Example 2: Construction of Variant Anti-CD3/Anti-Tumor Bispecific Antibodies

A panel of various anti-CD3/anti-tumor-associated antigen (TAA) bispecific antibodies (BsAbs) were constructed by genetic engineering. These BsAbs were derived from 4 anti-CD3 antibodies and 16 anti-TAA antibodies (CD20(CTAT01), CD19(CTAT02), EGFR(CTAT03), HER2(CTAT04), PSMA(CTAT05), CEA(CTAT06), EpCAM(CTAT07), FAP(CTAT08), PDL1(CTAT09), CD38(CTAT10), CD33(CTAT11), HGFR(CTAT12), CD47(CTAT13), TRAIL-R2(CTAT14), mesothelin (CTAT15) and GD2(CTAT16)). See Tables 1 and 2 above.

The BsAbs were produced via recombinant technology in mammalian host cells, collected, and examined by SDS-PAGE under reduced conditions and non-reduced conditions. Briefly, protein electrophoresis with 8% SDS-PAGE in non-reducing conditions and reducing conditions were performed to analyze the structure and molecular weight of various BsAbs comprising an anti-CD3 fragment and an anti-TAA fragment.

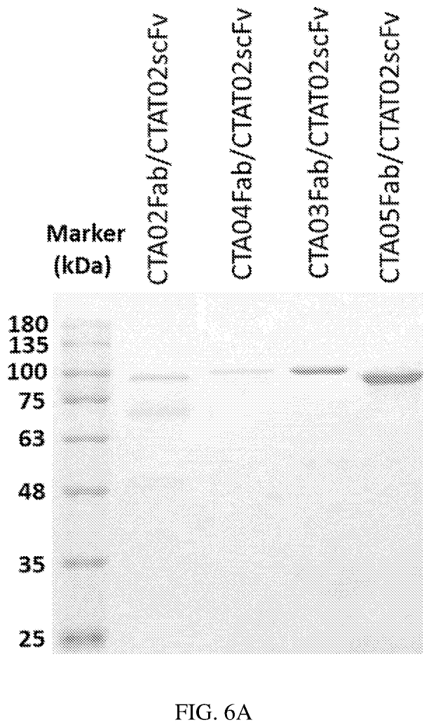

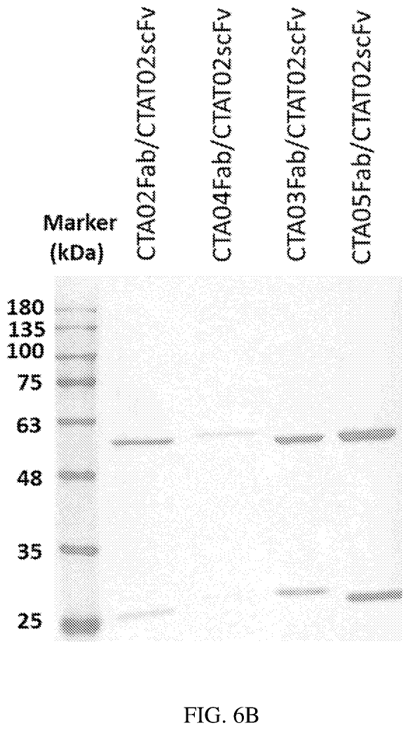

FIGS. 6A-6B show the expression and assembly of BsAbs each comprising a Fab of 4 different anti-CD3 antibody (CTA02, CTA03, CTA04, and CTA05; see Table 1 above) and an anti-CD19 scFv (CTAT02; see Table 2 above).

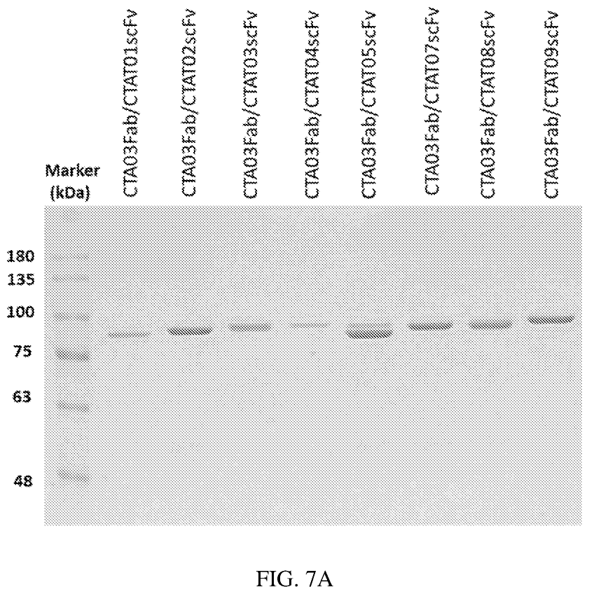

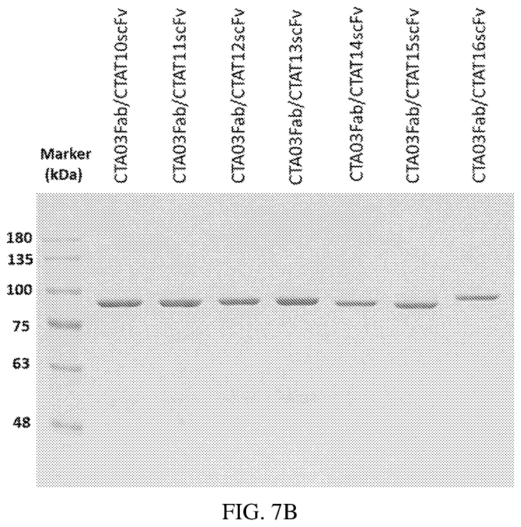

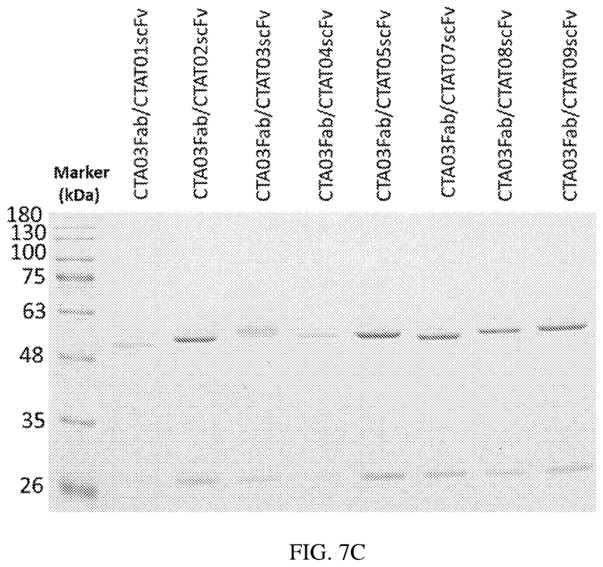

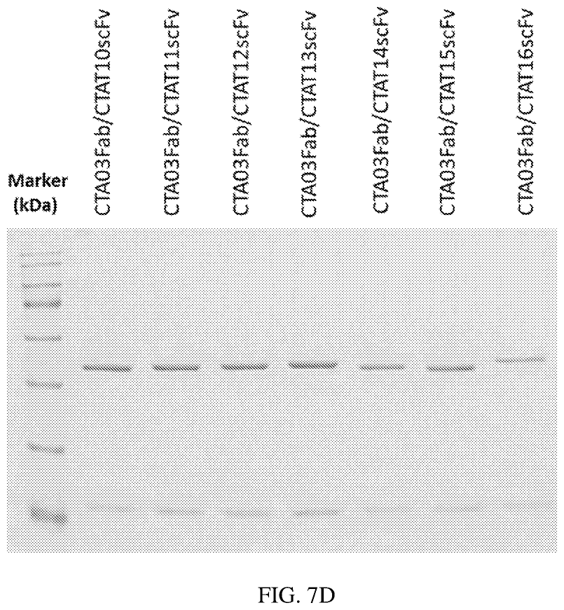

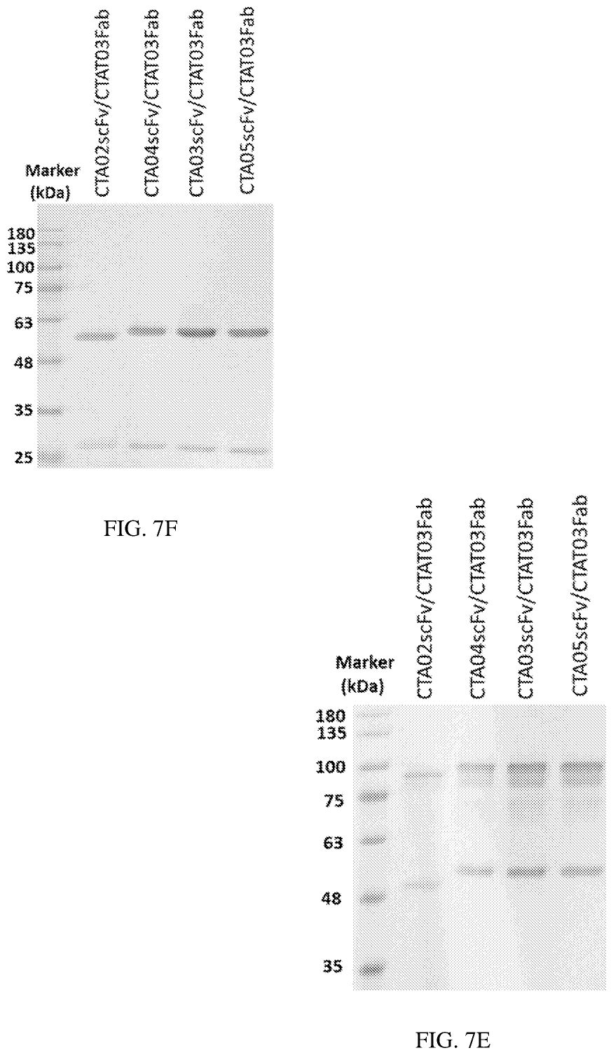

FIGS. 7A-7D show the expression and assembly of BsAbs each comprising an anti-CD3 Fab (CTA03; see Table 1 above) and a scFv of 16 different anti-tumor antibodies (CD20(CTAT01), CD19(CTAT02), EGFR (CTAT03), HER2(CTAT04), PSMA(CTAT05), CEA(CTAT06), EpCAM (CTAT07), FAP(CTAT08), PDLI(CTAT09), CD38(CTAT10), CD33(CTAT11), HGFR(CTAT12), CD47(CTAT13), TRAIL-R2(CTAT14), mesothelin (CTAT15) and GD2(CTAT16); see Table 2 above). FIGS. 7E and 7F show expression of BsAbs each comprising a scFv of one of the four anti-CD3 antibodies (CTA02-CTA05) and a Fab fragment of anti-EGFR CTAT03.

### Example 3: Anti-CD3/anti-TAA BsAbs Binds T Cells and Targets Tumor Cells Expressing the TAA

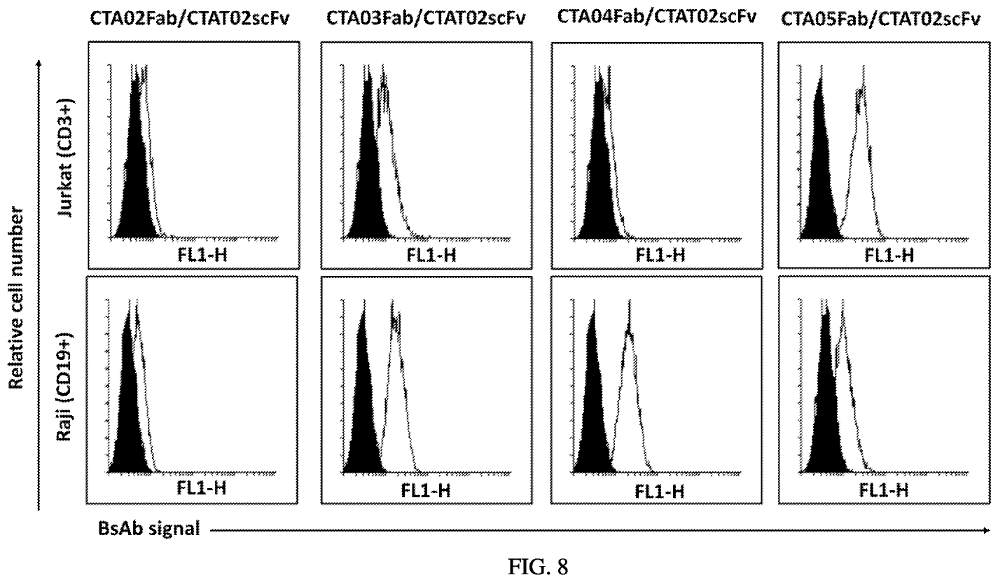

The binding activities of the various anti-CD3/anti-tumor BsAbs to T cells and tumor cells were analyzed using flow cytometry. The BsAbs specific to CD3 and CD19 (CTA02Fab/CTAT02scFv, CTA03Fab/CTAT02scFv, CTA04Fab/CTAT02scFv, and CTA05Fab/CTAT02scFv) all showed binding activity to CD3⁺ T cells (Jurkat) and CD19⁺ B cell lymphoma (Raji), indicating that T cells armed with such BsAbs could be used to target CD19+ disease cells such as CD19⁺ B cell lymphoma. FIG. 8.

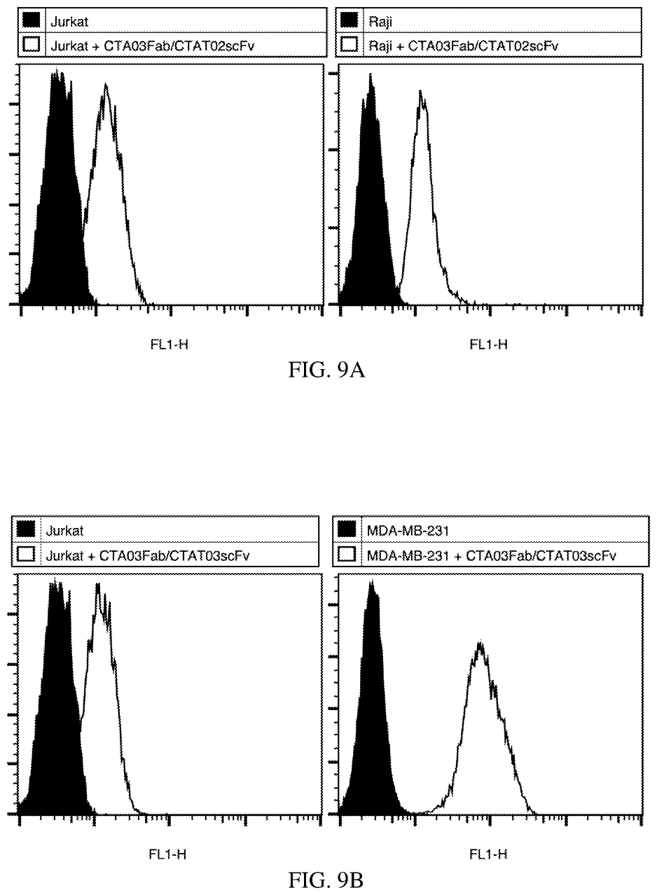

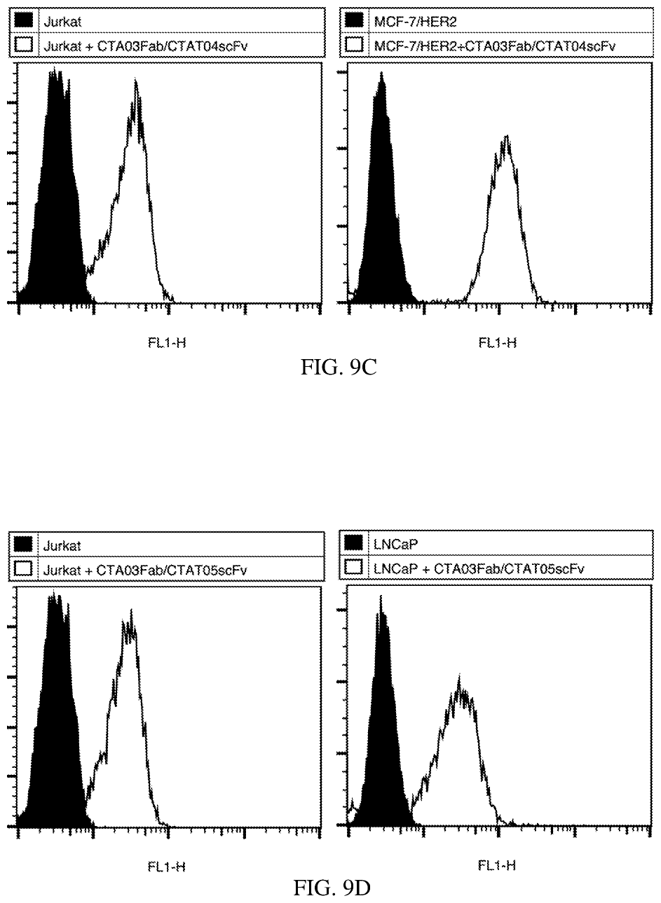

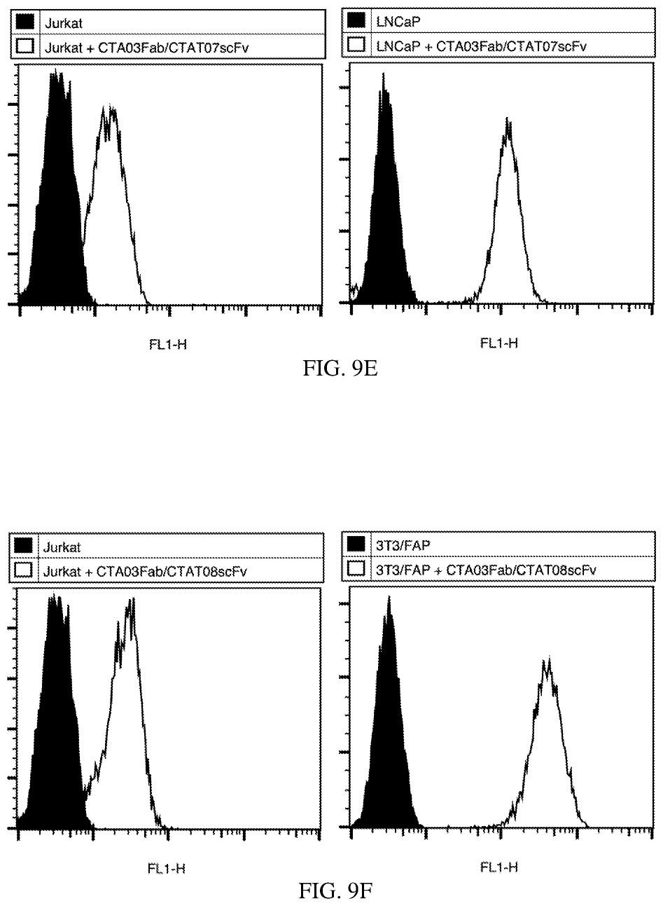

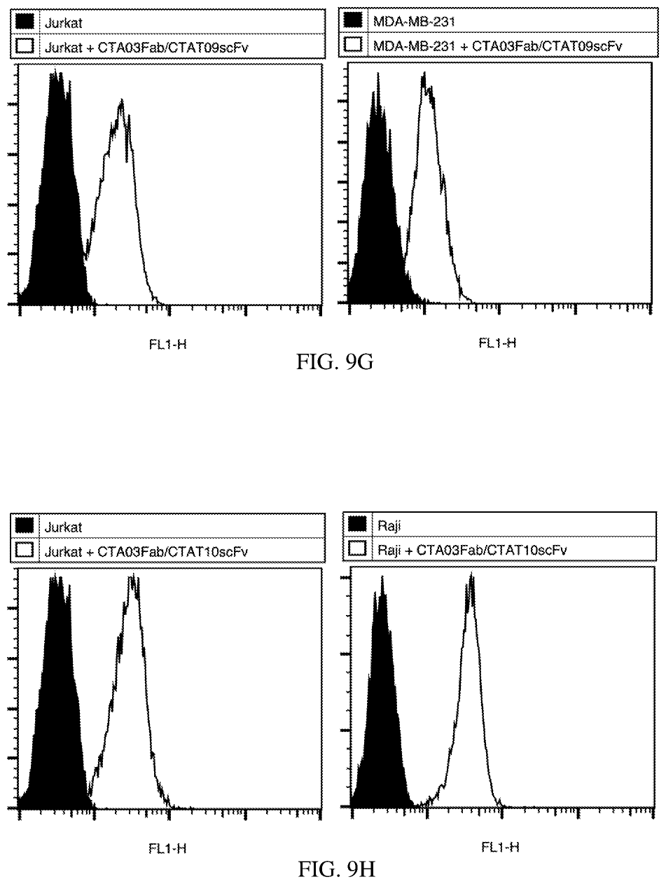

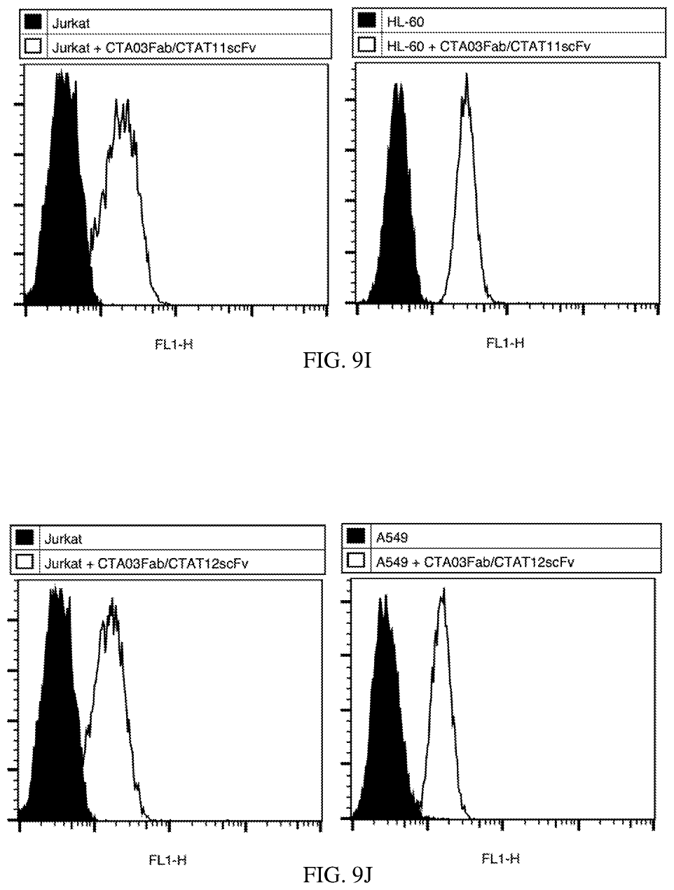

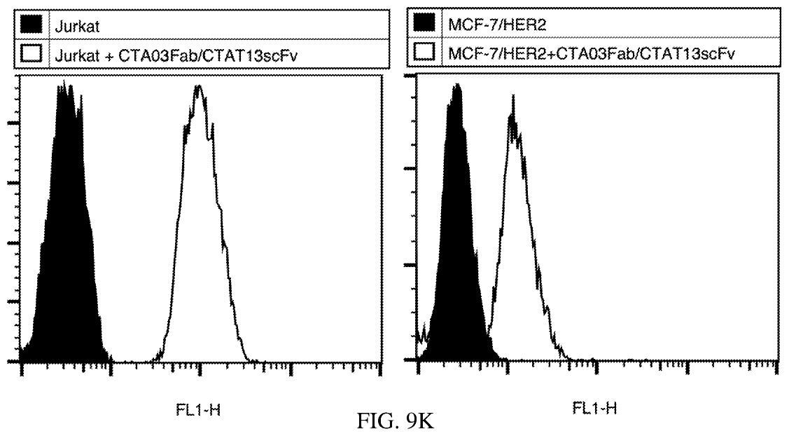

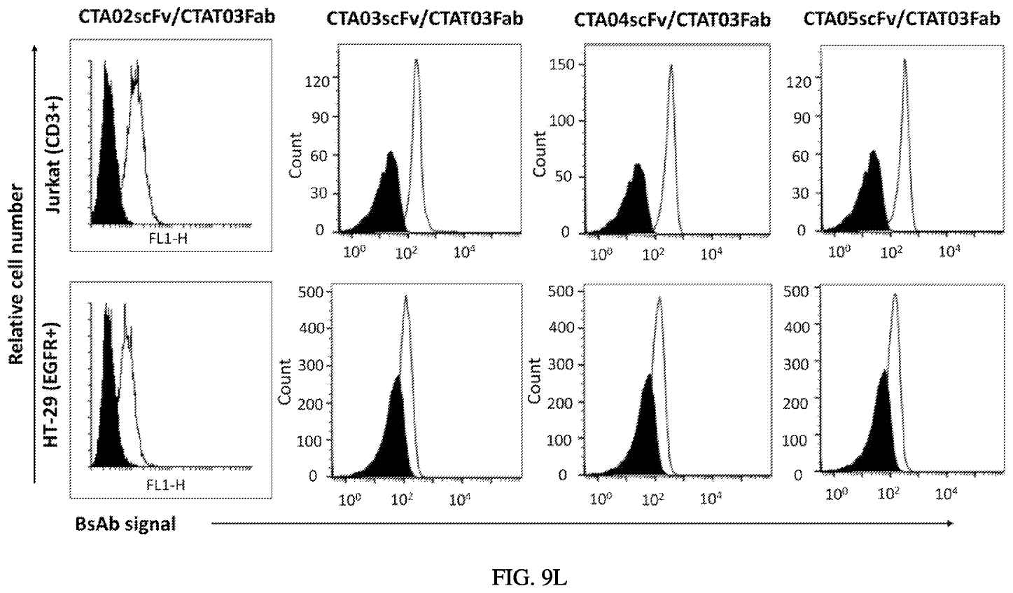

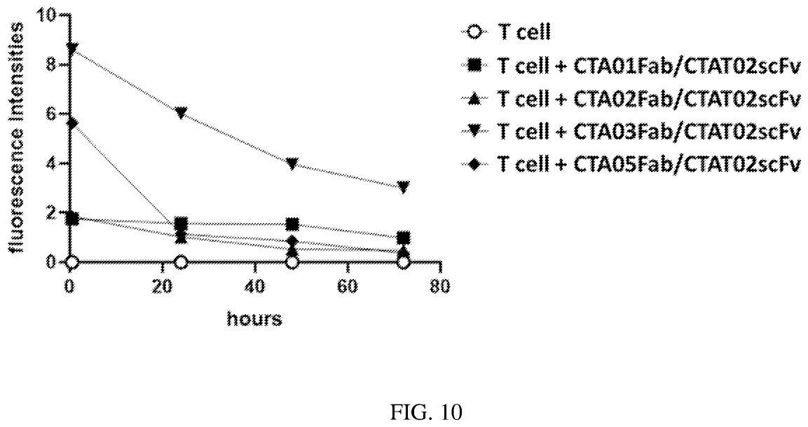

The binding activity of BsAbs having a binding fragment to other TAAs were also investigated. As shown in FIGS. 9A-10K:
CTA03Fab/CTAT02scFv showed binding activity to CD3⁺ T cells (Jurkat) and CD19⁺ B cell lymphoma (Raji) (FIG. 9A);
CTA03Fab/CTAT03scFv showed binding activity to CD3⁺ T cells (Jurkat) and EGFR⁺ triple negative breast cancer (MDA-MB-231) (FIG. 9B);
CTA03Fab/CTAT04scFv showed binding activity to CD3⁺ T cells (Jurkat) and HER2⁺ breast cancer (MCF7/HER2) (FIG. 9C);
CTA03Fab/CTAT05scFv showed binding activity to CD3⁺ T cells (Jurkat) and PSMA⁺ Prostate cancer (LNCaP) (FIG. 9D);
CTA03Fab/CTAT07scFv showed binding activity to CD3⁺ T cells (Jurkat) and EpCAM⁺ prostate cancer (LNCaP) (FIG. 9E);
CTA03Fab/CTAT08scFv showed binding activity to CD3⁺ T cells (Jurkat) and FAP⁺ mouse fibroblasts cell (3T3/FAP) (FIG. 9F);
CTA03Fab/CTAT09scFv showed binding activity to CD3⁺ T cells (Jurkat) and PDL1⁺ triple negative breast cancer (MDA-MB-231) (FIG. 9G);
CTA03Fab/CTAT10scFv showed binding activity to CD3⁺ T cells (Jurkat) and CD38⁺ B cell lymphoma (Raji) (FIG. 9H);
CTA03Fab/CTAT11scFv showed binding activity to CD3⁺ T cells (Jurkat) and CD33⁺ human acute myeloid leukemia (HL-60) (FIG. 9I);
CTA03Fab/CTAT12scFv showed binding activity to CD3⁺ T cells (Jurkat) and HGFR⁺ human lung carcinoma (A549) (FIG. 9J); and
CTA03Fab/CTAT13scFv showed binding activity to CD3⁺ T cells (Jurkat) and CD47⁺ breast cancer (MCF7/HER2) (FIG. 9K).

Further, the binding activities of various anti-CD3 scFv/anti-tumor Fab BsAbs to T cells and tumor cell were analyzed using flow cytometry. FIG. 9L shows the targeting ability against CD3+ T cells (Jurkat) and EGFR+ colon cancer (HT-29) of BsAbs consisting of a scFv of 4 different anti-CD3 antibody (CTA02, CTA03, CTA04, CTA05) and an anti-EGFR Fab (CTAT03). The results demonstrated that CTA02scFv/CTAT03Fab, CTA03scFv/CTAT03Fab, CTA04scFv/CTAT03Fab, and CTA05scFv/CTAT03Fab all possessed targeting abilities against CD3+ T cells (Jurkat) and EGFR+ colon cancer (HT-29).

### Example 4: Retention Ability of BsAb on T cell Surface

The retention time of BsAb on T cell surface was analyzed using an in vitro incubation platform. Briefly, human T cells were incubated with various anti-CD3Fab/anti-CD19scFv (CTA01Fab/CTAT02scFv, CTA02Fab/CTAT02scFv, CTA03Fab/CTAT02scFv and CTA05Fab/CTAT02scFv) for 1 hr, and then were cultured in medium for 5 min, 24, 48, and 72 hr. After the culture, the residual amount of BsAb on T cell surface was detected using flow cytometry. After 72 hrs, CTA01Fab/CTAT02scFv, CTA02Fab/CTAT02scFv, CTA03Fab/CTAT02scFv and CTA05Fab/CTAT02scFv were all detected on T cell surface, and CTA03Fab/CTAT02scFv had the highest retention amount on T cell surface. FIG. 10.

### Example 5: Preparation of Armed Immune Cells Using BsAbs via One-Step Incubation

Human peripheral blood mononuclear cells (PBMCs) from a healthy donor were cultured and differentiated into T cells in the presence of the OKT3 antibody, or in the presence of exemplary BsAbs disclosed herein (using CTA01Fab/CTAT02scFv, CTA02Fab/CTAT02scFv, CTA03Fab/CTAT02scFv and CTA05Fab/CTAT02scFv as examples). All groups were cultured under the same conditions (in an incubator with 5% CO₂ supply and a stable humidity level at 37° C.). After 7 days, all groups were analyzed using flow cytometry to measure production of BsAb armed-T cells.

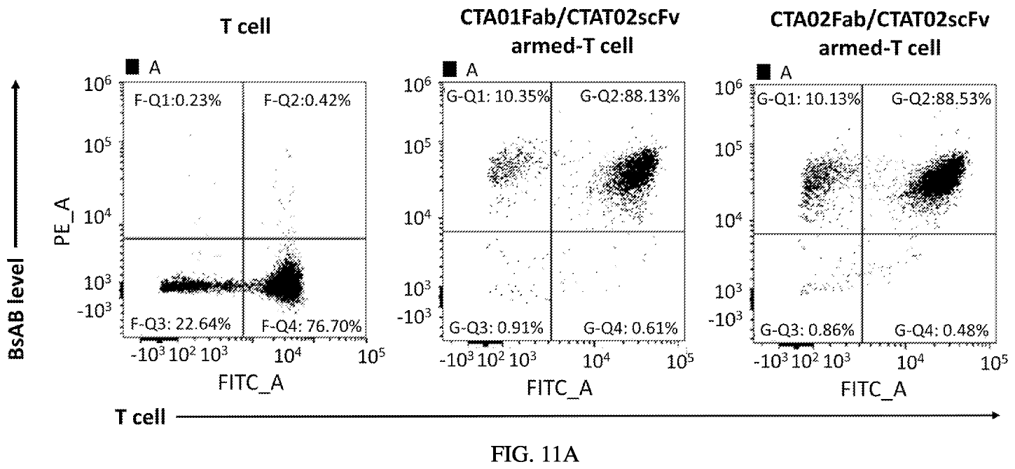

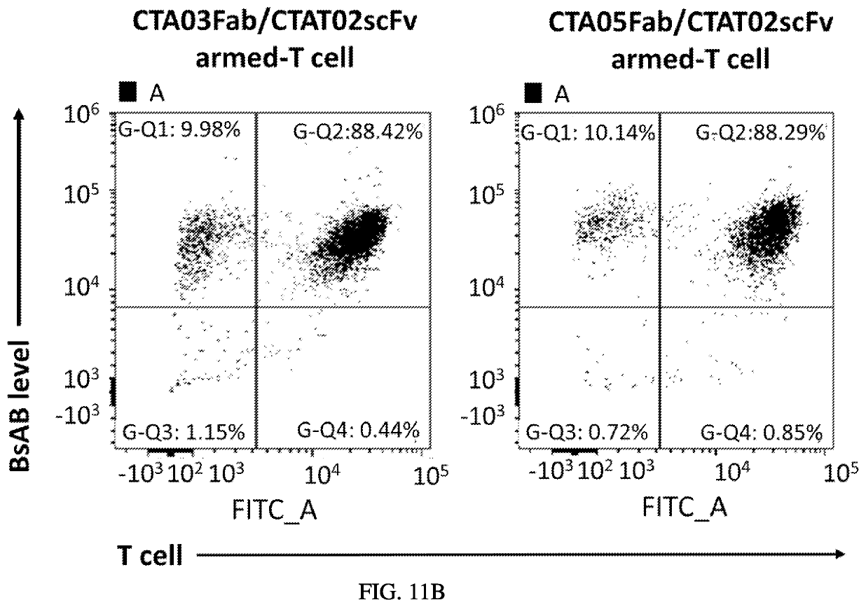

As shown in FIGS. 11A and 11B, the OKT3 anti-CD3 antibody induced differentiation of PBMCs into only normal T cells but not armed-T cells. By contrast, CTA01Fab/CTAT02scFv, CTA02Fab/CTAT02scFv, CTA03Fab/CTAT02scFv and CTA05Fab/CTAT02scFv BsAbs all successfully produced armed-T cells.

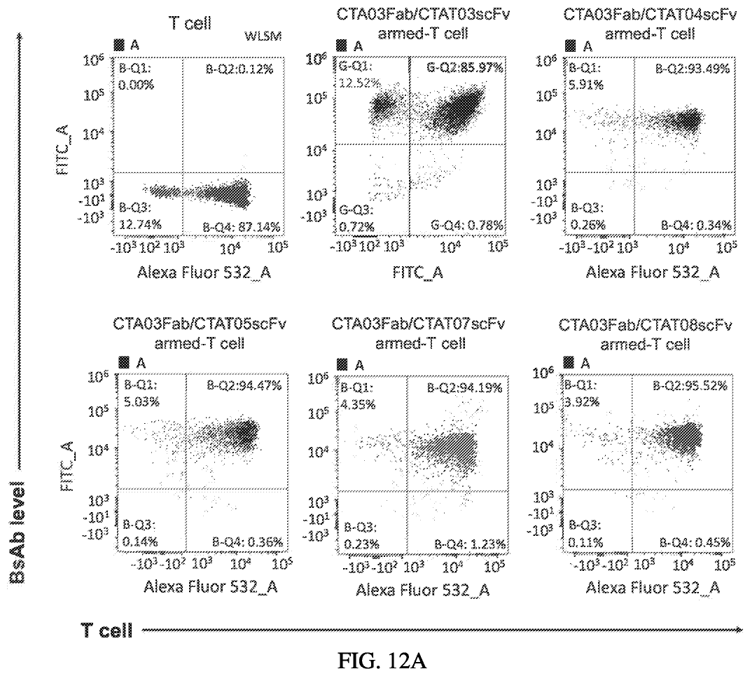

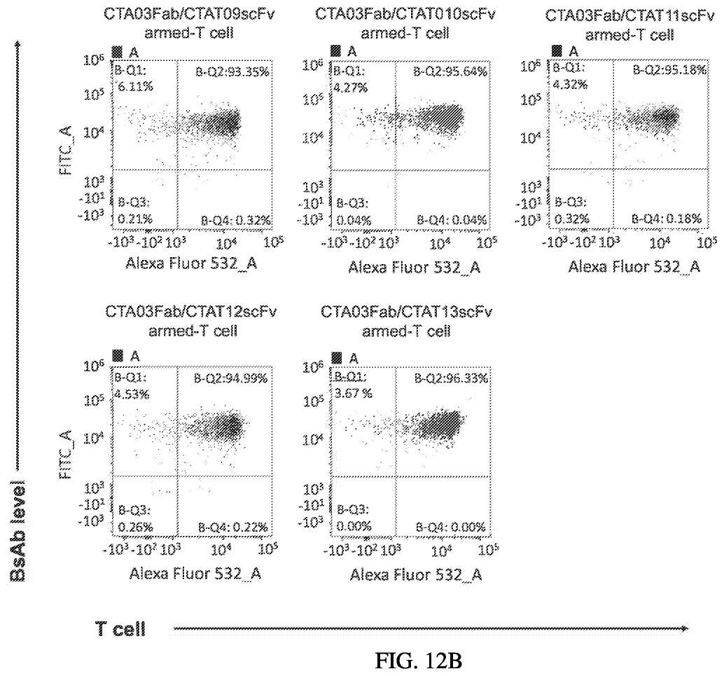

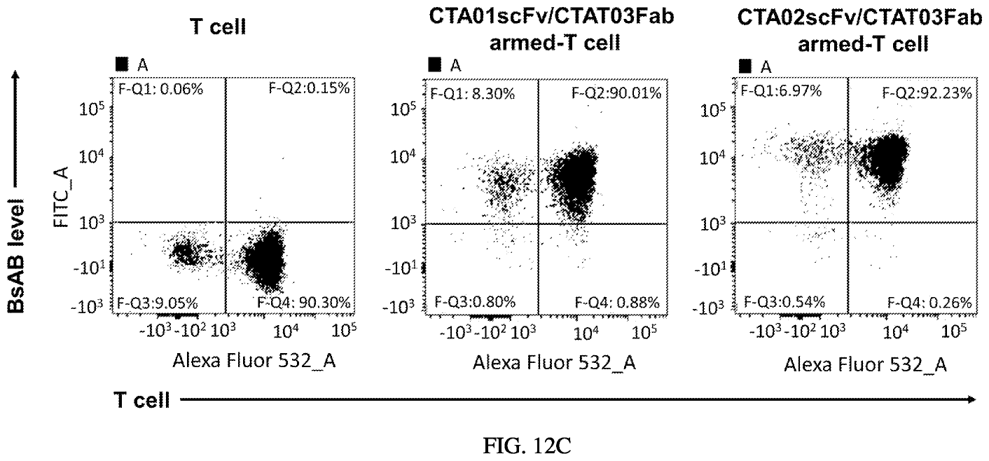

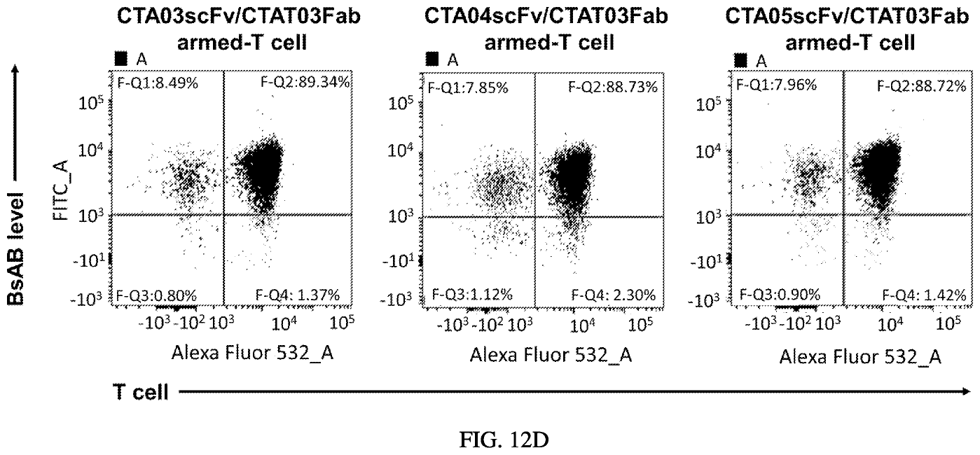

In another experiment, PBMCs were cultured and differentiated into T cells with OKT3 or exemplary anti-CD3 Fab/anti-Tumor scFv BsAbs (CTA03Fab/CTAT03scFv, CTA03Fab/CTAT04scFv, CTA03Fab/CTAT05scFv, CTA03Fab/CTAT07scFv, CTA03Fab/CTAT08scFv, CTA03Fab/CTAT9scFv, CTA03Fab/CTAT10scFv, CTA03Fab/CTAT11scFv, CTA03Fab/CTAT12scFv, and CTA03Fab/CTAT13scFv). All groups were cultured under the same conditions (in an incubator with 5% CO2 and a stable humidity level at)37° C. After 7 days, all groups were analyzed using flow cytometry to reveal whether BsAb armed-T cells were successfully generated. FIGS. 12A and 12B show that the OKT3 antibody led to differentiation of PBMCs into normal T cells, but not armed-T cells were produced. By contrast, CTA03Fab/CTAT03scFv, CTA03Fab/CTAT04scFv, CTA03Fab/CTAT05scFv, CTA03Fab/CTAT07scFv, CTA03Fab/CTAT08scFv, CTA03Fab/CTAT9scFv, CTA03Fab/CTAT10scFv, CTA03Fab/CTAT11scFv, CTA03Fab/CTAT12scFv, and CTA03Fab/CTAT13scFv BsAbs all led to production of armed-T cells.

Further, PBMCs were cultured and differentiated into T cells with OKT3 or various anti-CD3 scFv/anti-Tumor Fab BsAbs (CTA01scFv/CTAT03Fab, CTA02scFv/CTAT03Fab, CTA03scFv/CTAT03Fab, CTA04scFv/CTAT03Fab, and CTA05scFv/CTAT03Fab). All groups were cultured under the same conditions (in an incubator with 5% CO2 and a stable humidity level at)37° C. After 7 days, all groups were analyzed using flow cytometry to reveal whether BsAb armed-T cells were successfully generated. FIGS. 12C and 12D shows that the traditional OKT3 method caused PBMCs to differentiate only into normal T cells, but not armed-T cells. However, CTA01scFv/CTAT03Fab, CTA02scFv/CTAT03Fab, CTA03scFv/CTAT03Fab, CTA04scFv/CTAT03Fab, and CTA05scFv/CTAT03Fab BsAbs all successfully generated armed-T cells.

### Example 6: In Vitro Toxicity of BsAb-Armed T cells Against Tumor Cells

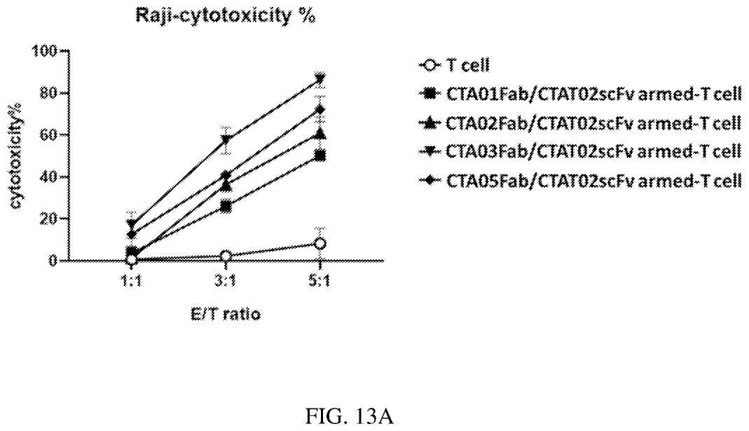

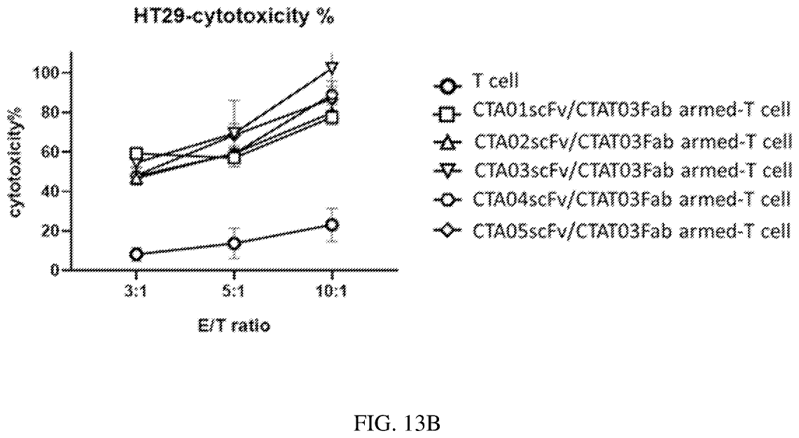

The cytotoxicity activity of T cells armed with anti-CD3/anti-CD19 BsAbs against CD19⁺ B cell lymphoma (Raji) were investigated in this example. The exemplary anti-CD3/anti-CD19 BsAbs disclosed herein, including CTA01Fab/CTAT02scFv, CTA02Fab/CTAT02scFv, CTA03Fab/CTAT02scFv and CTA05Fab/CTAT02scFv. T cells cultured with the OKT3 antibody was analyzed using an in vitro cytotoxicity assay. No significant cytotoxicity of T cells cultured with OKT3 against CD19⁺ B cell lymphoma (Raji) was observed. Differently, T cells cultured with CTA01Fab/CTAT02scFv, CTA02Fab/CTAT02scFv, CTA03Fab/CTAT02scFv, or CTA05Fab/CTAT02scFv efficiently killed CD19⁺ B cell lymphoma (Raji). Among the tested BsAbs, CTA03Fab/CTAT02scFv armed T cells had the best cytotoxic activity. FIG. 13A.

Further, the EGFR⁺ colon cancer (HT-29) killing activity of T cells armed with anti-CD3scFv/anti-EGFRFab BsAb consisting of 5 different anti-CD3 scFv (CTA01scFv/CTAT03Fab, CTA02scFv/CTAT03Fab, CTA03scFv/CTAT03Fab, CTA04scFv/CTAT03Fab, and CTA05scFv/CTAT03Fab) and T cells cultured with traditional OKT3 antibody was analyzed using an in vitro cytotoxicity assay. FIG. 13B shows that the T cells incubated with OKT3 did not efficiently kill EGFR⁺ colon cancer (HT-29), but the armed T cells cultured with CTA01scFv/CTAT03Fab, CTA02scFv/CTAT03Fab, CTA03scFv/CTAT03Fab, CTA04scFv/CTAT03Fab, and CTA05scFv/CTAT03Fab efficiently killed EGFR⁺ colon cancer (HT-29). FIG. 13B.

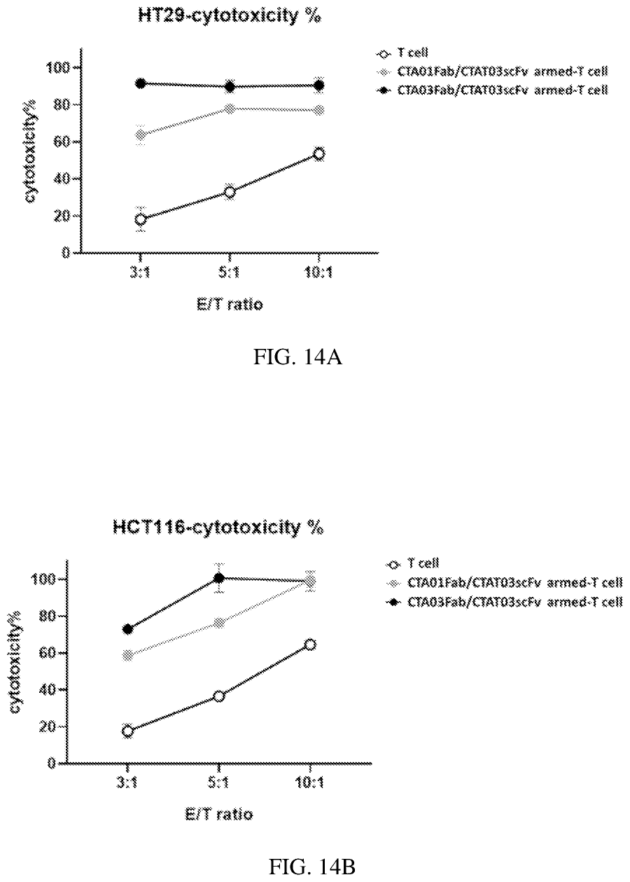

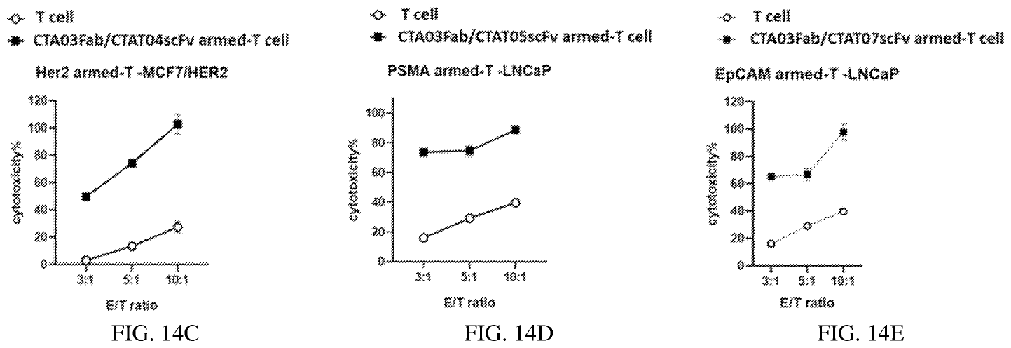

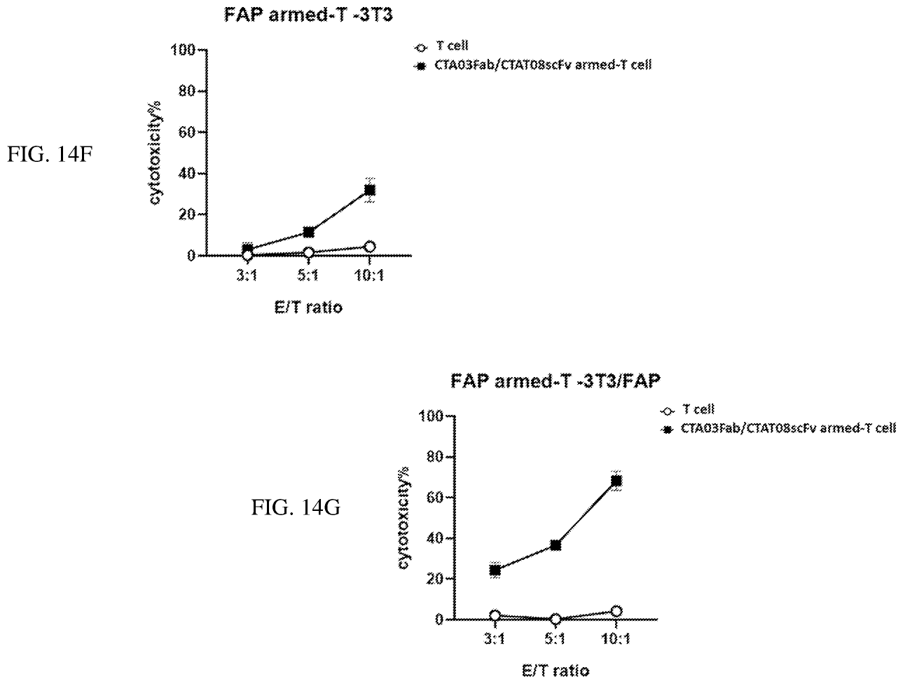

In another experiment, the tumor cell killing activity of armed T cells generated with various anti-CD3 Fab/anti-Tumor scFv BsAb (including CTA03Fab/CTAT03scFv, CTA03Fab/CTAT04scFv, CTA03Fab/CTAT05scFv, CTA03Fab/CTAT07scFv, CTA03Fab/CTAT08scFv, CTA03Fab/CTAT9scFv, CTA03Fab/CTAT10scFv, CTA03Fab/CTAT11scFv, CTA03Fab/CTAT12scFv and CTA03Fab/CTAT13scFv) were further analyzed. As shown in FIGS. 14A and 14B, CTA03Fab/CTAT03scFv armed-T cells efficiently killed EGFR⁺ colon cancer cells HT29 and HCT-116. FIG. 14C shows that CTA03Fab/CTAT04scFv armed-T cells efficiently killed HER2⁺ Breast cancer (MCF7/HER2). FIG. 14D shows that CTA03Fab/CTAT05scFv armed-T cells efficiently killed PSMA+Prostate cancer (LNCaP). FIG. 14E shows that CTA03Fab/CTAT07scFv armed-T cells efficiently killed EpCAM⁺ Prostate cancer (LNCaP). Further, FIGS. 14F-14G show that CTA03Fab/CTAT08scFv armed-T cells efficiently killed FAP⁺ mouse fibroblasts cell (3T3/FAP).

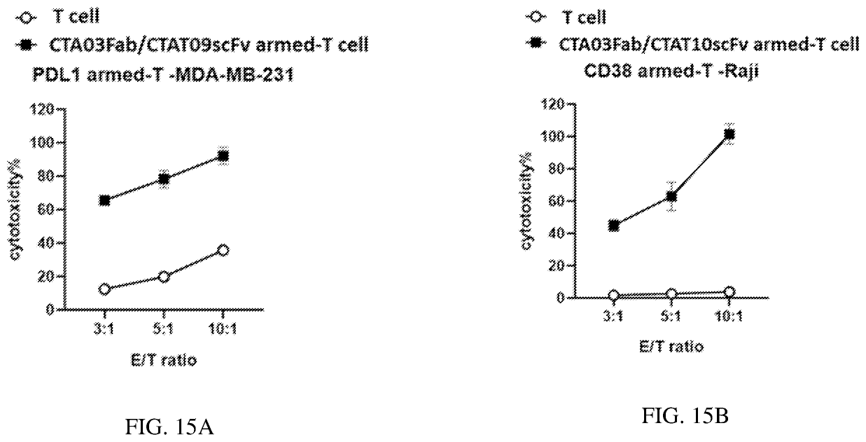

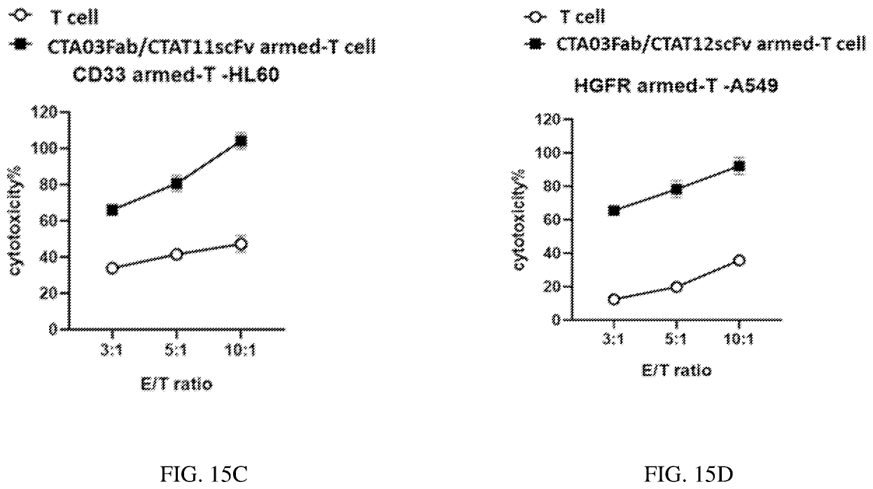

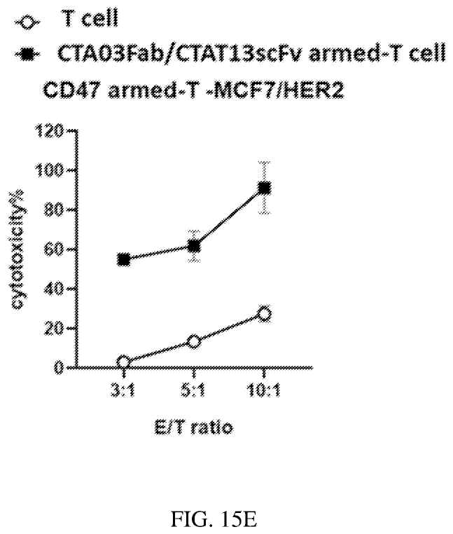

In addition, FIG. 15A shows that CTA03Fab/CTAT09scFv armed-T cells efficiently killed PDL1⁺ triple negative breast cancer (MDA-MB-231). FIG. 15B shows that CTA03Fab/CTAT10scFv armed-T cells efficiently killed CD38⁺ B cell lymphoma (Raji). FIG. 15C shows that CTA03Fab/CTAT1 1scFv armed-T cells efficiently killed CD33⁺ human acute myeloid leukemia (HL-60). FIG. 15D shows that CTA03Fab/CTAT12scFv armed-T cells efficiently killed HGFR⁺ human lung carcinoma (A549). FIG. 15E shows that CTA03Fab/CTAT13scFv armed-T cells efficiently killed CD47⁺ Breast cancer (MCF7/HER2).

### Example 7: In Vivo Anti-Cancer Activity of CTA03Fab/CTAT02scFv Armed-T Cells

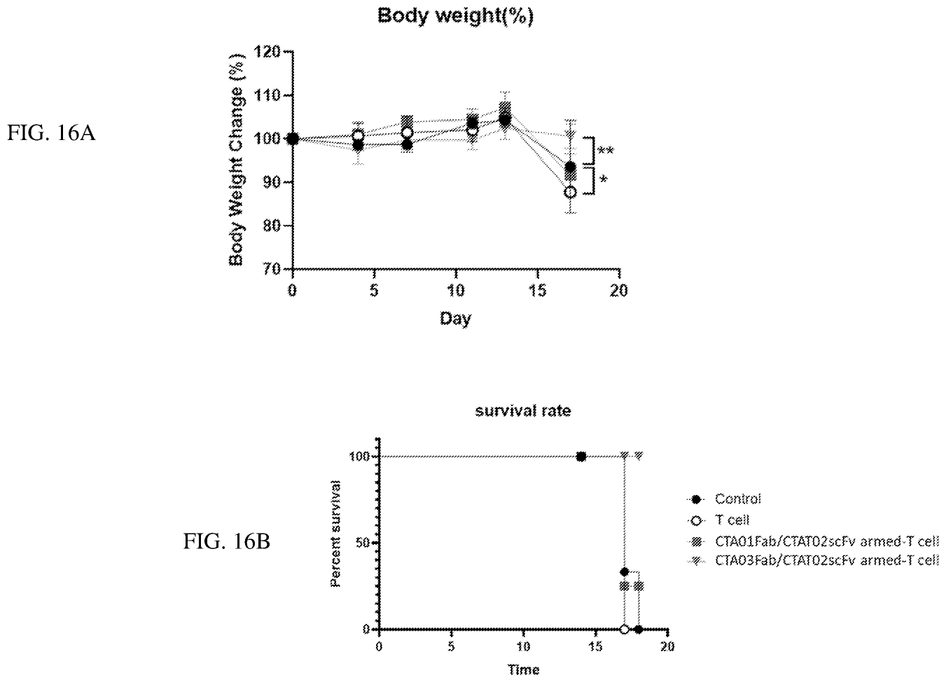

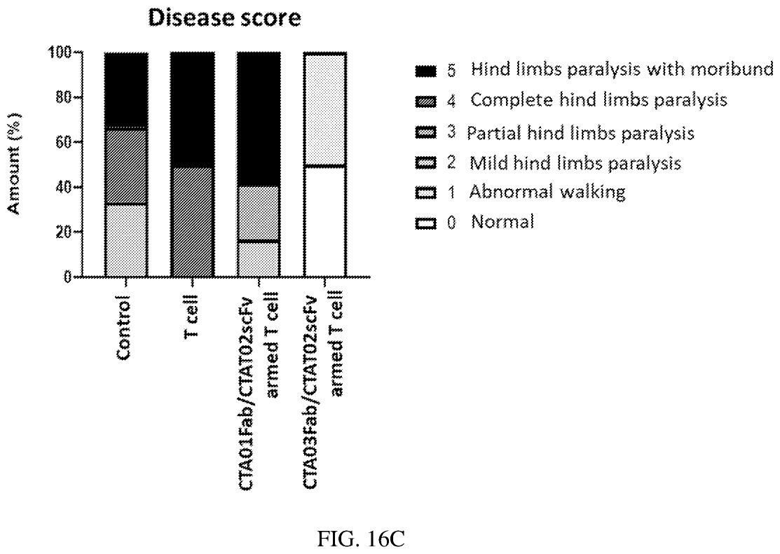

In vivo cancer inhibition of anti-CD3Fab/anti-CD19scFv (CTA01Fab/CTAT02scFv and CTA03Fab/CTAT02scFv) armed-T cells was evaluated. CTA01Fab/CTAT02scFv armed-T cells sand CTA03Fab/CTAT02scFv armed-T cells were i.v. injected into SCID mice bearing with B cell lymphoma (Raji). Body weight, survival rate and incidence of hindlimb paralysis were recorded. FIGS. 16A-16C show that CTA03Fab/CTAT02scFv armed-T cells had the best therapeutic efficacy to effectively inhibit cancer.

### Example 8: In Vivo Anti-Tumor Activity of CTA03Fab/CTAT03scFv Armed-T Cells and CTA03Fab/CTAT04scFv Armed-T Cells

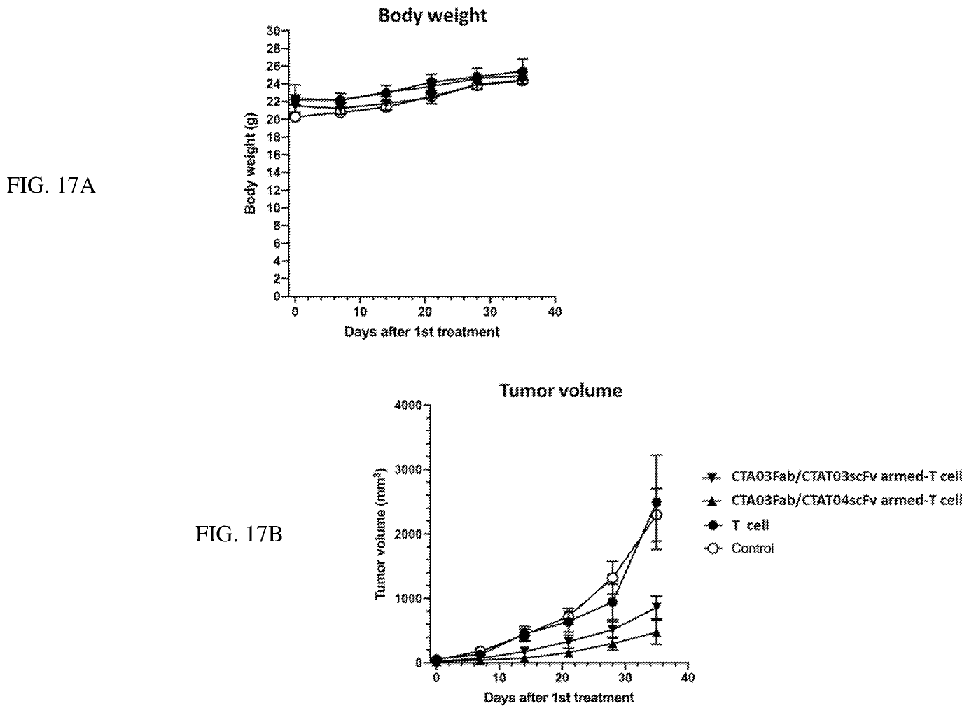

In vivo tumor inhibition of anti-CD3Fab/anti-EGFRscFv (CTA03Fab/CTAT03scFv) armed-T cells and anti-CD3Fab/anti-HER2scFv (CTA03Fab/CTAT04scFv) armed-T cells were evaluated. CTA03Fab/CTAT03scFv armed-T cells and CTA03Fab/CTAT04scFv armed-T cells were i.v. injected into patient-derived xenograft (PDX) mice models bearing with human triple-negative breast cancer. Body weight and tumor size were recorded. FIGS. 17A-17B show that CTA03Fab/CTAT03scFv armed-T cells and CTA03Fab/CTAT04scFv armed-T cells both efficiently inhibited the tumor growth of human triple-negative breast cancer.

### Example 9: One-Step Incubation for Producing Armed NKT Cells from PBMCs Using BsAbs

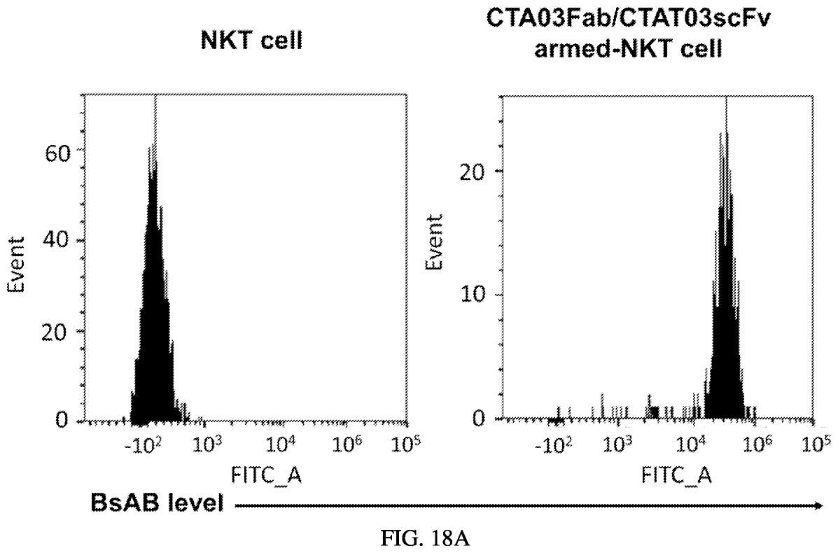

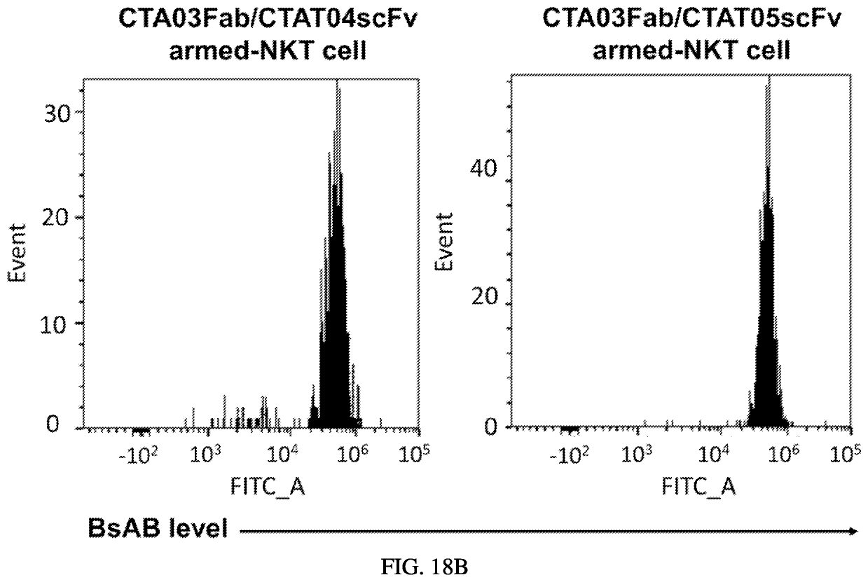

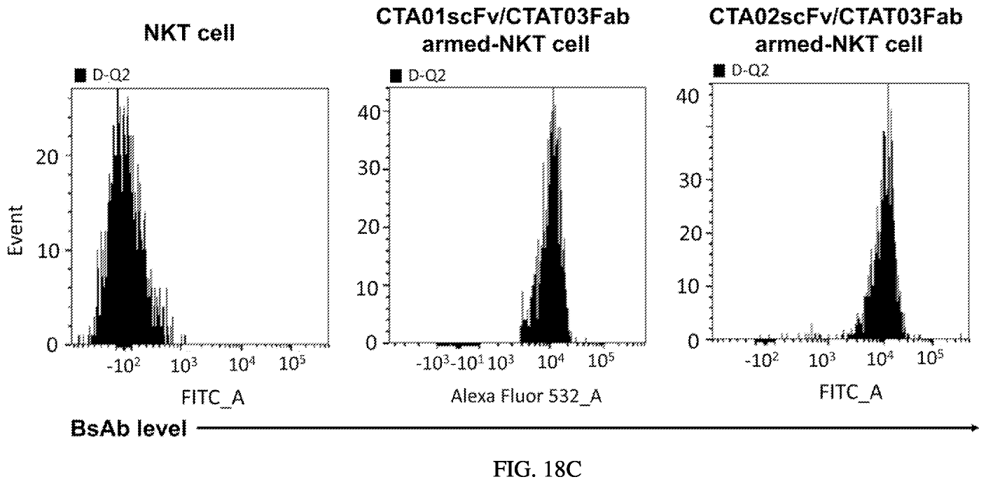

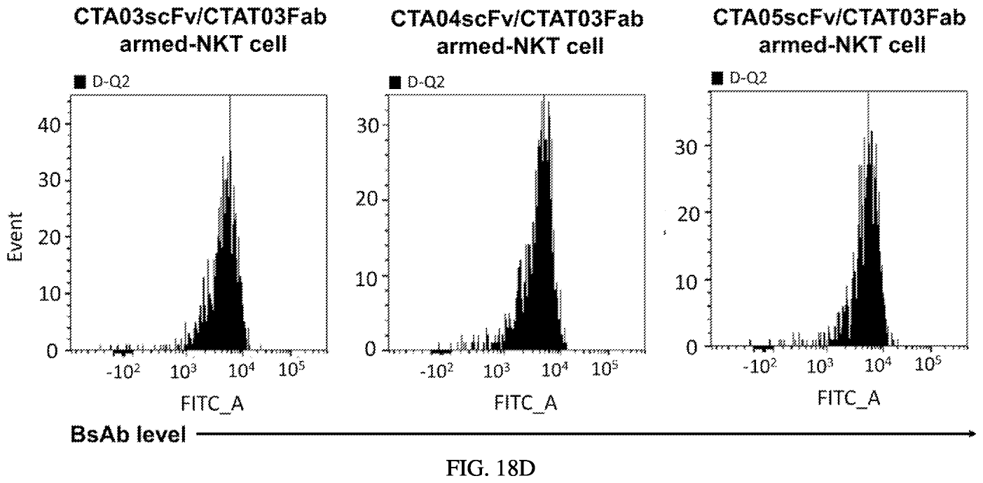

Human peripheral blood mononuclear cells (PBMCs) from a healthy donor were cultured and differentiated into NKT cells (CD8⁺ CD56⁺) with the OKT3 traditional method, or with CTA03Fab/CTAT03scFv, CTA03Fab/CTAT04scFv and CTA03Fab/CTAT05scFv BsAbs. All groups were cultured in the same environment (an incubator with 5% CO2 and a stable humidity level at)37° C. After 7 days, all groups were analyzed using flow cytometry to reveal whether BsAb armed-NKT cells were successfully generated. FIGS. 18A and 18B show that OKT3 method induced PBMCs to differentiate into only normal NKT cells, but not formation of armed-T cells. Differently, CTA03Fab/CTAT03scFv, CTA03Fab/CTAT04scFv and CTA03Fab/CTAT05scFv BsAbs all successfully generated armed-NKT cells.

In a similar assay, human peripheral blood mononuclear cells (PBMCs) from a healthy donor were cultured and differentiated into NKT cells (CD8⁺ CD56⁺) in the presence of the OKT antibody, or with CTA01scFv/CTAT03Fab, CTA02scFv/CTAT03Fab, CTA03scFv/CTAT03Fab, CTA04scFv/CTAT03Fab, and CTA05scFv/CTAT03Fab BsAbs. All groups were cultured in the same environment (an incubator with 5% CO2 and a stable humidity level at 37° C.). After 7 days, all groups were analyzed using flow cytometry to reveal whether BsAb armed-NKT cells were successfully generated. FIG. 34 shows that the traditional OKT3 method caused PBMCs to differentiate into only normal NKT cells, but not armed-T cells. However, CTA01scFv/CTAT03Fab, CTA02scFv/CTAT03Fab, CTA03scFv/CTAT03Fab, CTA04scFv/CTAT03Fab, and CTA05scFv/CTAT03Fab BsAbs all successfully generated armed-NKT cells.

### Example 10: Construction of CTA03Fab/CTAT02scFv BsAb With Point Mutations and Characterization Thereof

Point mutations were introduced into CTA03Fab/CTAT02scFv BsAb by genetic engineering, resulting in BsAbs CTA03-01Fab/CTAT02-01scFv (CTA03Fab(VLG58A)/CTAT02scFv(VLG42A)), CTA03-01Fab/CTAT02-02scFv (CTA03Fab(VLG58A)/CTAT02scFv(VLD41E)), CTA03-02Fab/CTAT02-02scFv (CTA03Fab(VLD57E)/CTAT02scFv(VLD41E)), and CTA03-02Fab/CTAT02-01scFv (CTA03Fab(VLD57E)/CTAT02scFv(VLG42A)). More specifically, point mutations G58A and D57E were introduced into the V_{L} of CTA03 and G42A and D41E were introduced into the V_{L} of CTAT02. See Table 2 above.

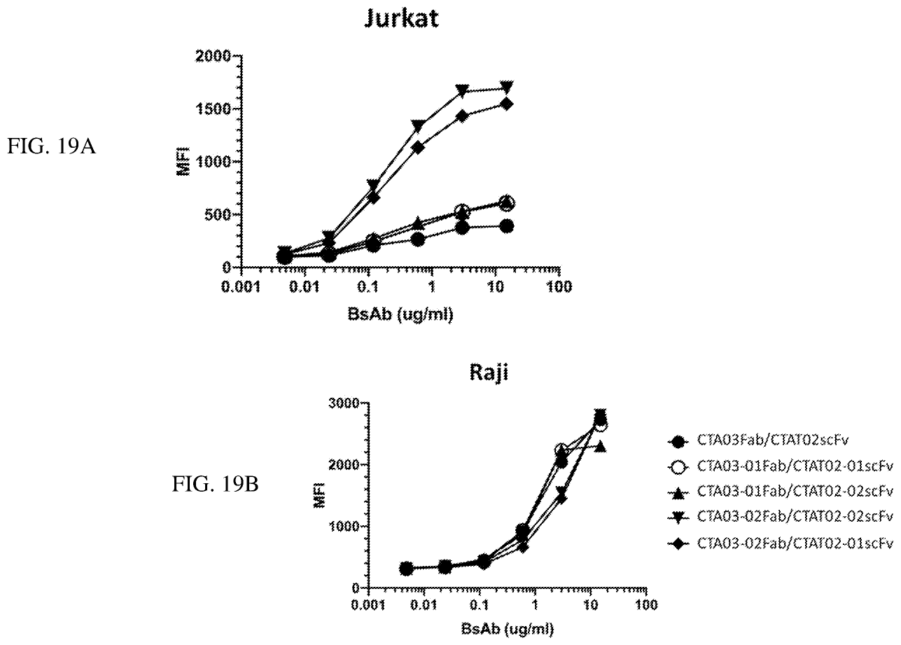

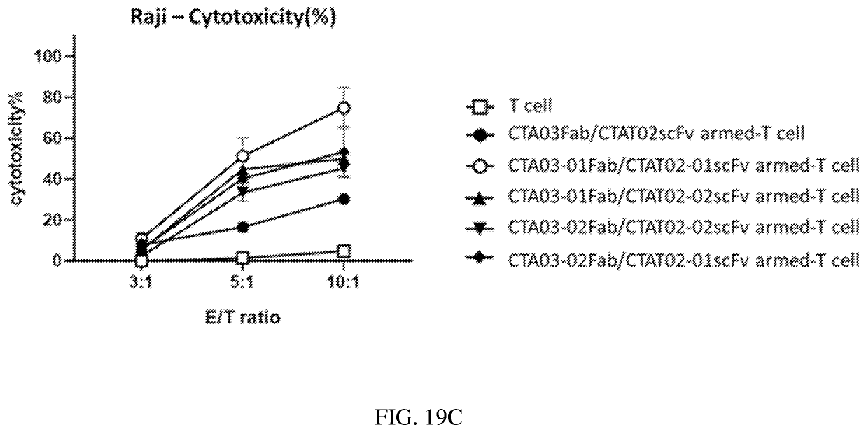

Binding abilities of the BsAbs against CD3⁺ T cells (Jurkat) and CD19⁺ B cells lymphoma (Raji) were analyzed using flow cytometry. FIGS. 19A-19B show that CTA03-01Fab/CTAT02-01scFv, CTA03-01Fab/CTAT02-02scFv, CTA03-02Fab/CTAT02-02scFv and CTA03-02Fab/CTAT02-01scFv BsAbs all possessed targeting abilities against CD3⁺ T cells (Jurkat) and CD19⁺ B cells lymphoma (Raji). Additionally, binding of the BsAbs to the target cells is in a dose-dependent manner.

The killing activity against CD19⁺ B cell lymphoma by the armed T cells generated with the point-mutant BsAbs (CTA03-01Fab/CTAT02-01scFv, CTA03-01Fab/CTAT02-02scFv, CTA03-02Fab/CTAT02-02scFv and CTA03-02Fab/CTAT02-01scFv) were compared with that of the original CTA03Fab/CTAT02scFv BsAb armed-T cells. FIG. 19C shows that T cells incubated with OKT3 did not show cytotoxicity against CD19⁺ B cell lymphoma (Raji). On the other hand, the armed-T cells cultured with CTA03-01Fab/CTAT02-01scFv, CTA03-01Fab/CTAT02-02scFv, CTA03-02Fab/CTAT02-02scFv or CTA03-02Fab/CTAT02-01scFv efficiently killed CD19⁺ B cell lymphoma (Raji), and the cytotoxicity were all better than the parent CTA03Fab/CTAT02scFv BsAb.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value. In some embodiments, the hinge domain is a hinge domain of a naturally occurring protein.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A bi-specific antibody, comprising:
(a) a first antigen binding fragment that binds human CD3, wherein the first antigen binding fragment comprises a first heavy chain comprising a first heavy chain variable region (V_{H}) and a first light chain comprising a first light chain variable region (V_{L}), wherein the first V_{H} comprises the same heavy chain complementary determining regions (CDRs) or no more than 5 amino acid variations relative to a first reference antibody and the first V_{L} comprises the same light chain CDRs or no more than 5 amino acid variations relative to the reference antibody, and wherein the first reference antibody is CTA.02, CTA.03, CTA.04, or CTA.05; and
(b) a second antigen binding fragment that binds a tumor associated antigen (TAA), wherein the second antigen binding fragment comprises a second heavy chain comprising a second heavy chain variable region (V_{H}) and a second light chain comprising a second light chain variable region (V_{L}).

2. The bi-specific antibody of claim 1, wherein the first heavy chain and the first light chain comprise the same V_{H} and V_{L} as the first reference antibody.

3. The bi-specific antibody of claim 1, wherein the second antigen binding fragment binds a TAA, which is CD20, CD19, EGFR, HER2, PSMA, CEA, EpCAM, FAP, PD-L1, CD38, CD33, cMET, CD47, TRAIL-R2, mesothelin, or GD2.

4. The bi-specific antibody of claim 3, wherein the second V_{H} comprises the same heavy chain complementary determining regions (CDRs) or no more than five amino acid variations relative to a second reference antibody and the second V_{L} comprises the same light chain CDRs or no more than 5 amino acid variations relative to the second reference antibody, and wherein the second reference antibody is CTAT.01, CTAT.02, CTAT.03, CTAT.04, CTAT.05, CTAT.06, CTAT.07, CTAT.08, CTAT.09, CTAT.10, CTAT.11, CTAT.12, CTAT.13, CTAT.14, CTAT.15, or CTAT.16.

5. The bi-specific antibody of claim 4, wherein the second antigen binding fragment comprises the same V_{H} and same V_{L} as the second reference antibody.

6. The bi-specific antibody of claim 1, wherein the first antigen binding fragment is a Fab fragment and the second antigen binding fragment is a single chain variable fragment (scFv), and wherein the Fab fragment comprises the first heavy chain, which comprises the first V_{H} and a CH1 fragment, and the first light chain, which comprises the first V_{L} and a light chain constant region.

7. The bi-specific antibody of claim 6, wherein the Fab fragment comprises the first heavy chain and the first light chain, which respectively comprise the amino acid sequences of (a) SEQ ID NO:10 and SEQ ID NO: 11, (b) SEQ ID NO: 23 and SEQ ID NO: 24, 25, or 228, (c) SEQ ID NO: 35 and SEQ ID NO: 36, or (d) SEQ ID NO: 46 and SEQ ID NO: 47.

8. The bi-specific antibody of claim 6, wherein the scFv of the second antigen binding fragment comprises the amino acid sequence of any one of SEQ ID NOs: 254-271.

9. The bi-specific antibody of claim 6, wherein the scFv is linked to the CH1 fragment or the light chain constant region via a peptide linker.

10. The bi-specific antibody of claim 7, wherein the bi-specific antibody comprises a first polypeptide comprising the first light chain and a second polypeptide comprising, from N-terminus to C-terminus, the first heavy chain, the peptide linker, and the scFv.

11. The bi-specific antibody of claim 8, wherein the first polypeptide comprises an amino acid sequence selected from the group consisting of SEQ ID NOs: 229-248.

12. The bi-specific antibody of claim 10, wherein the second polypeptide comprises an amino acid sequence selected from the group consisting of SEQ ID NOs: 24, 25, and 228.

13. The bi-specific antibody of claim 1, wherein the first antigen binding fragment is a single chain variable fragment (scFv) and the second antigen binding fragment is a Fab fragment, wherein the Fab fragment comprises the second heavy chain, which comprises the second V_{H} and a CH1 fragment, and the second light chain, which comprises the second V_{L} and a light chain constant region.

14. The bi-specific antibody of claim 13, wherein the scFv comprises the amino acid sequence of any one of SEQ ID NOs:250-253.

15. The bi-specific antibody of claim 13, wherein the Fab fragment comprises the first heavy chain and the first light chain, which respectively comprise the amino acid sequences of (1) SEQ ID NO:57 and SEQ ID NO: 58, (2) SEQ ID NO: 72 and SEQ ID NO: 73, (3) SEQ ID NO: 83 and SEQ ID NO: 84, (4) SEQ ID NO: 94 and SEQ ID NO: 95, (5) SEQ ID NO:105 and SEQ ID NO:106, (6) SEQ ID NO: 116 and SEQ ID NO:117, (7) SEQ ID NO: 127 and SEQ ID NO:128, (8) SEQ ID NO:138 and SEQ ID NO:139, (9) SEQ ID NO:149 and SEQ ID NO:150, (10) SEQ ID NO:160 and SEQ ID NO:161, (11) SEQ ID NO: 171 and SEQ ID NO:172, (12) SEQ ID NO:182 and SEQ ID NO:183, (13) SEQ ID NO:193 and SEQ ID NO:194, (14) SEQ ID NO:204 and SEQ ID NO:205, (15) SEQ ID NO:215 and SEQ ID NO:216, or (16) SEQ ID NO:226 and SEQ ID NO:227.

16. The bi-specific antibody of claim 13, wherein the scFv is linked to the CH1 fragment or the light chain constant region via a peptide linker, which optionally is at least 5 amino acids in length.

17. The bi-specific antibody of claim 1, wherein both the first antigen binding fragment and the second antigen binding fragment are scFv antibodies.

18. The bi-specific antibody of claim 13, wherein the bi-specific antibody comprises a polypeptide comprising the two scFv antibodies.

19. An armed immune cell, comprising an immune cell that expresses surface CD3, and a bi-specific antibody of claim 1, wherein the armed immune cell displays the bi-specific antibody on the surface via interaction between the first antigen binding fragment in the bi-specific antibody and the CD3 expressed by the immune cell.

20. The armed immune cell of claim 19, wherein the immune cell is a T cell, a B cell, a monocyte, a macrophage, or a combination thereof.

21. The armed immune cell of claim 20, wherein the T cell is a CD4+ T cell, a CD8+ T cell, a regulatory T cell, or a natural killer T cell.

22. The armed immune cell of claim 19, wherein the immune cell is a human immune cell.

23. The armed immune cell of claim 22, wherein the human immune cell is derived from a human donor.

24. A method of producing the armed immune cell of claim 19, comprising cultivating a cell population comprising the immune cells in the presence of the bi-specific antibody to allow for binding of the bi-specific antibody to the immune cells, thereby producing the armed immune cell.

25. The method of claim 24, wherein the cell population comprises T cells, B cells, monocytes, macrophages, or a combination thereof.

26. The method of claim 25, wherein the cell population comprises peripheral blood mononuclear cells (PBMCs) or immune cells derived from stem cells in vitro, and optionally wherein the stem cells are hematopoietic stem cells, umbilical cord blood stem cells, or induced pluripotent stem (iPS) cells.

27. The method of claim 24, wherein the cultivating step is performed in a culture medium comprising a cytokine, which optionally comprises interleukin 2 (IL-2), interleukin 7 (IL-7), transforming growth factor-beta (TGF-B), or a combination thereof.

28. A population of armed immune cells, which is produced by a method of claim 24.

29. A method for treating cancer, comprising administering to a subject in need thereof an effective amount of a population of armed immune cells set forth in claim 19, wherein the subject has or suspected of having a cancer that is positive with the TAA, to which the second antigen binding fragment of the bi-specific antibody binds.

30. The method of claim 29, wherein the subject is a human cancer patient.

31. The method of claim 29, wherein the armed immune cells are autologous to the subject.

32. The method of claim 29, wherein the armed immune cells are allogenic to the subject.

33. The method of claim 29, wherein the cancer is selected from the group consisting of melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, leukemia, lymphoma, and myeloma.

34. A nucleic acid or a set of nucleic acids, which encodes or collectively encodes a bi-specific antibody set forth in claim 1.

35. The nucleic acid or set of nucleic acids of claim 34, which is a vector or a set of vectors.

36. The nucleic acid or set of nucleic acids of claim 35, wherein the vector(s) is an expression vector(s).

37. A host cell, comprising the nucleic acid or set of nucleic acids set forth in claim 34.

38. The host cell of claim 37, wherein the host cell is a bacterial cell, a yeast cell, or a mammalian cell.

39. A method for producing a bi-specific antibody, comprising:
(i) culturing a host cell of claim 37 under conditions allowing for expressing of the bi-specific antibody; and
(ii) harvesting the bi-specific antibody.


SEQUENCE LISTING (249 sequences)

SEQ ID NO: 1 - protein, 5 aa, Artificial Sequence
  Synthetic
RYTMH

SEQ ID NO: 2 - protein, 17 aa, Artificial Sequence
  Synthetic
YINPSRGYTNYNQKFKD

SEQ ID NO: 3 - protein, 10 aa, Artificial Sequence
  Synthetic
YYDDHYCLDY

SEQ ID NO: 4 - protein, 119 aa, Artificial Sequence [sequence omitted]

SEQ ID NO: 5 - protein, 10 aa, Artificial Sequence
  Synthetic
SASSSVSYMN

SEQ ID NO: 6 - protein, 7 aa, Artificial Sequence
  Synthetic
DTSKLAS

SEQ ID NO: 7 - protein, 9 aa, Artificial Sequence
  Synthetic
QQWSSNPFT

SEQ ID NO: 8 - protein, 107 aa, Artificial Sequence
  Synthetic
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 9 - protein, 248 aa, Artificial Sequence
  Synthetic
GGGSGGGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 10 - protein, 216 aa, Artificial Sequence
  Synthetic
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKI

SEQ ID NO: 11 - protein, 213 aa, Artificial Sequence
  Synthetic
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 5 aa, Artificial Sequence
  Synthetic
SFPMA

SEQ ID NO: 13 - protein, 17 aa, Artificial Sequence
  Synthetic
TISTSGGRTYYRDSVKG

SEQ ID NO: 14 - protein, 10 aa, Artificial Sequence
  Synthetic
FRQYSGGFDY

SEQ ID NO: 15 - protein, 119 aa, Artificial Sequence [sequence omitted]

SEQ ID NO: 16 - protein, 13 aa, Artificial Sequence
  Synthetic
TLSSGNIENNYVH

SEQ ID NO: 17 - protein, 7 aa, Artificial Sequence
  Synthetic
DDDKRPD

SEQ ID NO: 18 - protein, 7 aa, Artificial Sequence
  Synthetic
DDDKRPE

SEQ ID NO: 19 - protein, 9 aa, Artificial Sequence
  Synthetic
HSYVSSFNV

SEQ ID NO: 20 - protein, 112 aa, Artificial Sequence
  Synthetic
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQ

SEQ ID NO: 21 - protein, 112 aa, Artificial Sequence
  Synthetic
DIQLTQPNSVSTSLGSTVKLSCTLSSGNIENNYVHWYQLYEGRSPTTMIYDDDKRPDAVPDRFSGSIDRSSNSAFLTIHNVAIEDEAIYFCHSYVSSFNVFGGGTKLTVLRQ

SEQ ID NO: 22 - protein, 253 aa, Artificial Sequence
  Synthetic
GGGSGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSGGGGSGGGGSGGGGSDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQ

SEQ ID NO: 23 - protein, 222 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 24 - protein, 216 aa, Artificial Sequence
  Synthetic
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 216 aa, Artificial Sequence
  Synthetic
DIQLTQPNSVSTSLGSTVKLSCTLSSGNIENNYVHWYQLYEGRSPTTMIYDDDKRPEGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 5 aa, Artificial Sequence
  Synthetic
RYTMH

SEQ ID NO: 27 - protein, 17 aa, Artificial Sequence
  Synthetic
YINPSRGYTNYNQKVKD

SEQ ID NO: 28 - protein, 10 aa, Artificial Sequence
  Synthetic
YYDDHYCLDY

SEQ ID NO: 29 - protein, 119 aa, Artificial Sequence [sequence omitted]

SEQ ID NO: 30 - protein, 10 aa, Artificial Sequence
  Synthetic
SASSSVSYMN

SEQ ID NO: 31 - protein, 7 aa, Artificial Sequence
  Synthetic
DTSKLAS

SEQ ID NO: 32 - protein, 9 aa, Artificial Sequence
  Synthetic
QQWSSNPFT

SEQ ID NO: 33 - protein, 107 aa, Artificial Sequence
  Synthetic
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 34 - protein, 248 aa, Artificial Sequence
  Synthetic
GGGSGGGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 35 - protein, 222 aa, Artificial Sequence
  Synthetic
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 213 aa, Artificial Sequence
  Synthetic
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 5 aa, Artificial Sequence
  Synthetic
SYTMH

SEQ ID NO: 38 - protein, 17 aa, Artificial Sequence
  Synthetic
YINPRSGYTHYNQKLKD

SEQ ID NO: 39 - protein, 11 aa, Artificial Sequence
  Synthetic
SAYYDYDGFAY

SEQ ID NO: 40 - protein, 120 aa, Artificial Sequence [sequence omitted]

SEQ ID NO: 41 - protein, 10 aa, Artificial Sequence
  Synthetic
SASSSVSYMN

SEQ ID NO: 42 - protein, 7 aa, Artificial Sequence
  Synthetic
DTSKLAS

SEQ ID NO: 43 - protein, 9 aa, Artificial Sequence
  Synthetic
QQWSSNPPT

SEQ ID NO: 44 - protein, 106 aa, Artificial Sequence [sequence omitted]

SEQ ID NO: 45 - protein, 248 aa, Artificial Sequence
  Synthetic
GGGSGGGQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIK

SEQ ID NO: 46 - protein, 220 aa, Artificial Sequence
  Synthetic
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPCSRSTSESTAALGCLVKDYFPEPVTVSWNSGALTSGVHTFPAVLQSSGLYSLSSVAIVTVPSSNFGTQTYTCNVDHKPSNTKVDKTV

SEQ ID NO: 47 - protein, 213 aa, Artificial Sequence
  Synthetic
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 48 - protein, 5 aa, Artificial Sequence
  Synthetic
SYNMH

SEQ ID NO: 49 - protein, 17 aa, Artificial Sequence
  Synthetic
AIYPGNGDTSYNQKFKG

SEQ ID NO: 50 - protein, 12 aa, Artificial Sequence
  Synthetic
STYYGGDWYFNV

SEQ ID NO: 51 - protein, 121 aa, Artificial Sequence [sequence omitted]

SEQ ID NO: 52 - protein, 10 aa, Artificial Sequence
  Synthetic
RASSSVSYIH

SEQ ID NO: 53 - protein, 7 aa, Artificial Sequence
  Synthetic
ATSNLAS

SEQ ID NO: 54 - protein, 9 aa, Artificial Sequence
  Synthetic
QQWTSNPPT

SEQ ID NO: 55 - protein, 106 aa, Artificial Sequence [sequence omitted]

SEQ ID NO: 56 - protein, 249 aa, Artificial Sequence
  Synthetic
GGGSGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 57 - protein, 224 aa, Artificial Sequence
  Synthetic
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSC

SEQ ID NO: 58 - protein, 213 aa, Artificial Sequence
  Synthetic
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 5 aa, Artificial Sequence
  Synthetic
DYGVS

SEQ ID NO: 60 - protein, 16 aa, Artificial Sequence
  Synthetic
VIWGSETTYYNSALKS

SEQ ID NO: 61 - protein, 12 aa, Artificial Sequence
  Synthetic
HYYYGGSYAMDY

SEQ ID NO: 62 - protein, 120 aa, Artificial Sequence
  Synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 63 - protein, 11 aa, Artificial Sequence
  Synthetic
RASQDISKYLN

SEQ ID NO: 64 - protein, 7 aa, Artificial Sequence
  Synthetic
HTSRLHS

SEQ ID NO: 65 - protein, 9 aa, Artificial Sequence
  Synthetic
QQGNTLPYT

SEQ ID NO: 66 - protein, 107 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 67 - protein, 107 aa, Artificial Sequence
  Synthetic
DIQMTQTTSSLSASLGDRVTISCRASQDISKYLNWYQQKPDATVKLLIYHTSRLHSGVPSRFSGSGSGTDYSLTISNLEQEDIATYFCQQGNTLPYTFGGGTKLEIT

SEQ ID NO: 68 - protein, 107 aa, Artificial Sequence
  Synthetic
DIQMTQTTSSLSASLGDRVTISCRASQDISKYLNWYQQKPEGTVKLLIYHTSRLHSGVPSRFSGSGSGTDYSLTISNLEQEDIATYFCQQGNTLPYTFGGGTKLEIT

SEQ ID NO: 69 - protein, 249 aa, Artificial Sequence
  Synthetic
GGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 70 - protein, 249 aa, Artificial Sequence
  Synthetic
GGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDATVKLLIYHTSRLHSGVPSRFSGSGSGTDYSLTISNLEQEDIATYFCQQGNTLPYTFGGGTKLEIT

SEQ ID NO: 71 - protein, 249 aa, Artificial Sequence
  Synthetic
GGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPEGTVKLLIYHTSRLHSGVPSRFSGSGSGTDYSLTISNLEQEDIATYFCQQGNTLPYTFGGGTKLEIT

SEQ ID NO: 72 - protein, 225 aa, Artificial Sequence
  Synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 73 - protein, 214 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 74 - protein, 5 aa, Artificial Sequence
  Synthetic
NYGVH

SEQ ID NO: 75 - protein, 16 aa, Artificial Sequence
  Synthetic
VIWSGGNTDYNTPFTS

SEQ ID NO: 76 - protein, 11 aa, Artificial Sequence
  Synthetic
ALTYYDYEFAY

SEQ ID NO: 77 - protein, 119 aa, Artificial Sequence
  Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 78 - protein, 11 aa, Artificial Sequence
  Synthetic
RASQSIGTNIH

SEQ ID NO: 79 - protein, 7 aa, Artificial Sequence
  Synthetic
YASESIS

SEQ ID NO: 80 - protein, 9 aa, Artificial Sequence
  Synthetic
QQNNNWPTT

SEQ ID NO: 81 - protein, 107 aa, Artificial Sequence
  Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 82 - protein, 248 aa, Artificial Sequence
  Synthetic
GGGSGGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 83 - protein, 224 aa, Artificial Sequence
  Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 84 - protein, 214 aa, Artificial Sequence
  Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 85 - protein, 5 aa, Artificial Sequence
  Synthetic
DTYIH

SEQ ID NO: 86 - protein, 17 aa, Artificial Sequence
  Synthetic
RIYPTNGYTRYADSVKG

SEQ ID NO: 87 - protein, 11 aa, Artificial Sequence
  Synthetic
WGGDGFYAMDY

SEQ ID NO: 88 - protein, 120 aa, Artificial Sequence [sequence omitted]

SEQ ID NO: 89 - protein, 11 aa, Artificial Sequence
  Synthetic
RASQDVNTAVA

SEQ ID NO: 90 - protein, 7 aa, Artificial Sequence
  Synthetic
SASFLYS

SEQ ID NO: 91 - protein, 9 aa, Artificial Sequence
  Synthetic
QQHYTTPPT

SEQ ID NO: 92 - protein, 107 aa, Artificial Sequence [sequence omitted]

SEQ ID NO: 93 - protein, 249 aa, Artificial Sequence
  Synthetic
GGGSGGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 94 - protein, 225 aa, Artificial Sequence
  Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 95 - protein, 214 aa, Artificial Sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 96 - protein, 5 aa, Artificial Sequence
  Synthetic
EYTIH

SEQ ID NO: 97 - protein, 17 aa, Artificial Sequence
  Synthetic
NINPNNGGTTYNQKFED

SEQ ID NO: 98 - protein, 6 aa, Artificial Sequence
  Synthetic
GWNFDY

SEQ ID NO: 99 - protein, 115 aa, Artificial Sequence
  Synthetic
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSS

SEQ ID NO: 100 - protein, 11 aa, Artificial Sequence
  Synthetic
KASQDVGTAVD

SEQ ID NO: 101 - protein, 7 aa, Artificial Sequence
  Synthetic
WASTRHT

SEQ ID NO: 102 - protein, 9 aa, Artificial Sequence
  Synthetic
QQYNSYPLT

SEQ ID NO: 103 - protein, 107 aa, Artificial Sequence
  Synthetic
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 104 - protein, 244 aa, Artificial Sequence
  Synthetic
GGGSGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGGGGSGGGGSGGGGSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 105 - protein, 220 aa, Artificial Sequence
  Synthetic
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 106 - protein, 214 aa, Artificial Sequence
  Synthetic
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 107 - protein, 5 aa, Artificial Sequence
  Synthetic
EYGMN

SEQ ID NO: 108 - protein, 17 aa, Artificial Sequence
  Synthetic
WINTKSGEATYVEEFKG

SEQ ID NO: 109 - protein, 12 aa, Artificial Sequence
  Synthetic
WDFYDYVDEAMY

SEQ ID NO: 110 - protein, 121 aa, Artificial Sequence
  Synthetic
QVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSS

SEQ ID NO: 111 - protein, 11 aa, Artificial Sequence
  Synthetic
KASQTVSANVA

SEQ ID NO: 112 - protein, 7 aa, Artificial Sequence
  Synthetic
LASYRYR

SEQ ID NO: 113 - protein, 10 aa, Artificial Sequence
  Synthetic
HQYYTYPLFT

SEQ ID NO: 114 - protein, 108 aa, Artificial Sequence
  Synthetic
DIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIK

SEQ ID NO: 115 - protein, 251 aa, Artificial Sequence
  Synthetic
GGGSGGGQVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSGGGGSGGGGSGGGGSDIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIK

SEQ ID NO: 116 - protein, 226 aa, Artificial Sequence
  Synthetic
QVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 117 - protein, 215 aa, Artificial Sequence
  Synthetic
DIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 118 - protein, 5 aa, Artificial Sequence
  Synthetic
NYGMN

SEQ ID NO: 119 - protein, 17 aa, Artificial Sequence
  Synthetic
WINTYTGESTYADSFKG

SEQ ID NO: 120 - protein, 7 aa, Artificial Sequence
  Synthetic
FAIKGDY

SEQ ID NO: 121 - protein, 116 aa, Artificial Sequence
  Synthetic
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 122 - protein, 16 aa, Artificial Sequence
  Synthetic
RSTKSLLHSNGITYLY

SEQ ID NO: 123 - protein, 7 aa, Artificial Sequence
  Synthetic
QMSNLAS

SEQ ID NO: 124 - protein, 9 aa, Artificial Sequence
  Synthetic
AQNLEIPRT

SEQ ID NO: 125 - protein, 112 aa, Artificial Sequence
  Synthetic
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 126 - protein, 250 aa, Artificial Sequence
  Synthetic
GGGSGGGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSGGGGSGGGGSGGGGSDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 127 - protein, 221 aa, Artificial Sequence
  Synthetic
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 128 - protein, 219 aa, Artificial Sequence
  Synthetic
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 129 - protein, 5 aa, Artificial Sequence
  Synthetic
EYTIH

SEQ ID NO: 130 - protein, 17 aa, Artificial Sequence
  Synthetic
GINPNNGIPNYNQKFKG

SEQ ID NO: 131 - protein, 15 aa, Artificial Sequence
  Synthetic
RRIAYGYDEGHAMDY

SEQ ID NO: 132 - protein, 124 aa, Artificial Sequence
  Synthetic
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 133 - protein, 17 aa, Artificial Sequence
  Synthetic
KSSQSLLYSRNQKNYLA

SEQ ID NO: 134 - protein, 7 aa, Artificial Sequence
  Synthetic
WASTRES

SEQ ID NO: 135 - protein, 9 aa, Artificial Sequence
  Synthetic
QQYFSYPLT

SEQ ID NO: 136 - protein, 113 aa, Artificial Sequence
  Synthetic
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 137 - protein, 259 aa, Artificial Sequence
  Synthetic
GGGSGGGQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGGSGGGGSGGGGSDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 138 - protein, 229 aa, Artificial Sequence
  Synthetic
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 139 - protein, 220 aa, Artificial Sequence
  Synthetic
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 140 - protein, 5 aa, Artificial Sequence
  Synthetic
DSWIH

SEQ ID NO: 141 - protein, 17 aa, Artificial Sequence
  Synthetic
WISPYGGSTYYADSVKG

SEQ ID NO: 142 - protein, 9 aa, Artificial Sequence
  Synthetic
RHWPGGFDY

SEQ ID NO: 143 - protein, 118 aa, Artificial Sequence [sequence omitted]

SEQ ID NO: 144 - protein, 11 aa, Artificial Sequence
  Synthetic
RASQDVSTAVA

SEQ ID NO: 145 - protein, 7 aa, Artificial Sequence
  Synthetic
SASFLYS

SEQ ID NO: 146 - protein, 9 aa, Artificial Sequence
  Synthetic
QQYLYHPAT

SEQ ID NO: 147 - protein, 107 aa, Artificial Sequence [sequence omitted]

SEQ ID NO: 148 - protein, 247 aa, Artificial Sequence
  Synthetic
GGGSGGGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 149 - protein, 223 aa, Artificial Sequence
  Synthetic
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 150 - protein, 214 aa, Artificial Sequence
  Synthetic
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 5 aa, Artificial Sequence
  Synthetic
SFAMS

SEQ ID NO: 152 - protein, 17 aa, Artificial Sequence
  Synthetic
AISGSGGGTYYADSVKG

SEQ ID NO: 153 - protein, 13 aa, Artificial Sequence
  Synthetic
DKILWFGEPVFDY

SEQ ID NO: 154 - protein, 122 aa, Artificial Sequence [sequence omitted]

SEQ ID NO: 155 - protein, 11 aa, Artificial Sequence
  Synthetic
RASQSVSSYLA

SEQ ID NO: 156 - protein, 7 aa, Artificial Sequence
  Synthetic
DASNRAT

SEQ ID NO: 157 - protein, 9 aa, Artificial Sequence
  Synthetic
QQRSNWPPT

SEQ ID NO: 158 - protein, 107 aa, Artificial Sequence [sequence omitted]

SEQ ID NO: 159 - protein, 251 aa, Artificial Sequence
  Synthetic
GGGSGGGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 160 - protein, 227 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 161 - protein, 214 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 5 aa, Artificial Sequence
  Synthetic
DSNIH

SEQ ID NO: 163 - protein, 17 aa, Artificial Sequence
  Synthetic
YIYPYNGGTDYNQKFKN

SEQ ID NO: 164 - protein, 7 aa, Artificial Sequence
  Synthetic
GNPWLAY

SEQ ID NO: 165 - protein, 116 aa, Artificial Sequence [sequence omitted]

SEQ ID NO: 166 - protein, 15 aa, Artificial Sequence
  Synthetic
RASESLDNYGIRFLT

SEQ ID NO: 167 - protein, 7 aa, Artificial Sequence
  Synthetic
AASNQGS

SEQ ID NO: 168 - protein, 9 aa, Artificial Sequence
  Synthetic
QQTKEVPWS

SEQ ID NO: 169 - protein, 111 aa, Artificial Sequence [sequence omitted]

SEQ ID NO: 170 - protein, 249 aa, Artificial Sequence
  Synthetic
GGGSGGGEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 171 - protein, 214 aa, Artificial Sequence
  Synthetic
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 172 - protein, 218 aa, Artificial Sequence
  Synthetic
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 5 aa, Artificial Sequence
  Synthetic
SYWLH

SEQ ID NO: 174 - protein, 17 aa, Artificial Sequence
  Synthetic
MIDPSNSDTRFNPNFKD

SEQ ID NO: 175 - protein, 10 aa, Artificial Sequence
  Synthetic
YRSYVTPLDY

SEQ ID NO: 176 - protein, 119 aa, Artificial Sequence [sequence omitted]

SEQ ID NO: 177 - protein, 17 aa, Artificial Sequence
  Synthetic
KSSQSLLYTSSQKNYLA

SEQ ID NO: 178 - protein, 7 aa, Artificial Sequence
  Synthetic
WASTRES

SEQ ID NO: 179 - protein, 9 aa, Artificial Sequence
  Synthetic
QQYYAYPWT

SEQ ID NO: 180 - protein, 113 aa, Artificial Sequence [sequence omitted]

SEQ ID NO: 181 - protein, 254 aa, Artificial Sequence
  Synthetic
GGGSGGGEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIK

SEQ ID NO: 182 - protein, 224 aa, Artificial Sequence
  Synthetic
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 183 - protein, 220 aa, Artificial Sequence
  Synthetic
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 5 aa, Artificial Sequence
  Synthetic
NYNMH

SEQ ID NO: 185 - protein, 17 aa, Artificial Sequence
  Synthetic
TIYPGNDDTSYNQKFKD

SEQ ID NO: 186 - protein, 8 aa, Artificial Sequence
  Synthetic
GGYRAMDY

SEQ ID NO: 187 - protein, 117 aa, Artificial Sequence
  Synthetic
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 188 - protein, 16 aa, Artificial Sequence
  Synthetic
RSSQSIVYSNGNTYLG

SEQ ID NO: 189 - protein, 7 aa, Artificial Sequence
  Synthetic
KVSNRFS

SEQ ID NO: 190 - protein, 9 aa, Artificial Sequence
  Synthetic
FQGSHVPYT

SEQ ID NO: 191 - protein, 112 aa, Artificial Sequence
  Synthetic
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 192 - protein, 251 aa, Artificial Sequence
  Synthetic
GGGSGGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGGGGSGGGGSGGGGSDIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 193 - protein, 222 aa, Artificial Sequence
  Synthetic
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 194 - protein, 219 aa, Artificial Sequence
  Synthetic
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 7 aa, Artificial Sequence
  Synthetic
SGDYFWS

SEQ ID NO: 196 - protein, 16 aa, Artificial Sequence
  Synthetic
HIHNSGTTYYNPSLKS

SEQ ID NO: 197 - protein, 12 aa, Artificial Sequence
  Synthetic
DRGGDYYYGMDV

SEQ ID NO: 198 - protein, 122 aa, Artificial Sequence
  Synthetic
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSS

SEQ ID NO: 199 - protein, 12 aa, Artificial Sequence
  Synthetic
RASQGISRSYLA

SEQ ID NO: 200 - protein, 7 aa, Artificial Sequence
  Synthetic
GASSRAT

SEQ ID NO: 201 - protein, 9 aa, Artificial Sequence
  Synthetic
QQFGSSPWT

SEQ ID NO: 202 - protein, 108 aa, Artificial Sequence
  Synthetic
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 203 - protein, 252 aa, Artificial Sequence
  Synthetic
GGGSGGGQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSGGGGSGGGGSGGGGSEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 204 - protein, 227 aa, Artificial Sequence
  Synthetic
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 205 - protein, 215 aa, Artificial Sequence
  Synthetic
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 7 aa, Artificial Sequence
  Synthetic
SGSYYWS

SEQ ID NO: 207 - protein, 16 aa, Artificial Sequence
  Synthetic
YIYYSGSTNYNPSLKS

SEQ ID NO: 208 - protein, 9 aa, Artificial Sequence
  Synthetic
EGKNGAFDI

SEQ ID NO: 209 - protein, 119 aa, Artificial Sequence
  Synthetic
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 210 - protein, 11 aa, Artificial Sequence
  Synthetic
RASQSISSYLN

SEQ ID NO: 211 - protein, 7 aa, Artificial Sequence
  Synthetic
AASSLQS

SEQ ID NO: 212 - protein, 9 aa, Artificial Sequence
  Synthetic
QQSYSTPLT

SEQ ID NO: 213 - protein, 107 aa, Artificial Sequence
  Synthetic
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 214 - protein, 248 aa, Artificial Sequence
  Synthetic
GGGSGGGQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 215 - protein, 224 aa, Artificial Sequence
  Synthetic
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 216 - protein, 214 aa, Artificial Sequence
  Synthetic
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 5 aa, Artificial Sequence
  Synthetic
GYNMN

SEQ ID NO: 218 - protein, 17 aa, Artificial Sequence
  Synthetic
AIDPYYGGTSYNQKFKG

SEQ ID NO: 219 - protein, 4 aa, Artificial Sequence
  Synthetic
GMEY

SEQ ID NO: 220 - protein, 113 aa, Artificial Sequence [sequence omitted]

SEQ ID NO: 221 - protein, 16 aa, Artificial Sequence
  Synthetic
RSSQSLVHRNGNTYLH

SEQ ID NO: 222 - protein, 7 aa, Artificial Sequence
  Synthetic
KVSNRFS

SEQ ID NO: 223 - protein, 10 aa, Artificial Sequence
  Synthetic
SQSTHVPPLT

SEQ ID NO: 224 - protein, 113 aa, Artificial Sequence [sequence omitted]

SEQ ID NO: 225 - protein, 248 aa, Artificial Sequence
  Synthetic
GGGSGGGEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 226 - protein, 218 aa, Artificial Sequence
  Synthetic
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 227 - protein, 220 aa, Artificial Sequence
  Synthetic
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 216 aa, Artificial Sequence
  Synthetic
DIQLTQPNSVSTSLGSTVKLSCTLSSGNIENNYVHWYQLYEGRSPTTMIYDDDKRPDAVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 229 - protein, 471 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 230 - protein, 471 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 231 - protein, 470 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 232 - protein, 471 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 233 - protein, 466 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGGGGSGGGGSGGGGSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 234 - protein, 473 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSGGGGSGGGGSGGGGSDIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIK

SEQ ID NO: 235 - protein, 472 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSGGGGSGGGGSGGGGSDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 236 - protein, 481 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGGSGGGGSGGGGSDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 237 - protein, 469 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 238 - protein, 473 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 239 - protein, 471 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 240 - protein, 476 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIK

SEQ ID NO: 241 - protein, 473 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGGGGSGGGGSGGGGSDIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 242 - protein, 474 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSGGGGSGGGGSGGGGSEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 243 - protein, 470 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 244 - protein, 470 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 245 - protein, 471 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDATVKLLIYHTSRLHSGVPSRFSGSGSGTDYSLTISNLEQEDIATYFCQQGNTLPYTFGGGTKLEIT

SEQ ID NO: 246 - protein, 471 aa, Artificial Sequence
  Synthetic
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPEGTVKLLIYHTSRLHSGVPSRFSGSGSGTDYSLTISNLEQEDIATYFCQQGNTLPYTFGGGTKLEIT

SEQ ID NO: 247 - protein, 471 aa, Artificial Sequence; identical to SEQ ID NO: 246
  Synthetic

SEQ ID NO: 248 - protein, 471 aa, Artificial Sequence; identical to SEQ ID NO: 245
  Synthetic

SEQ ID NO: 249 - protein, 112 aa, Artificial Sequence
  Synthetic
DIQLTQPNSVSTSLGSTVKLSCTLSSGNIENNYVHWYQLYEGRSPTTMIYDDDKRPEGVPDRFSGSIDRSSNSAFLTIHNVAIEDEAIYFCHSYVSSFNVFGGGTKLTVLRQ